(19) United States
(12) Patent Application Publication
(10) Pub. No.: US 2012/0294826 A1
(43) Pub. Date: Nov. 22, 2012

(54) METHODS, KITS AND COMPOSITIONS FOR AMELIORATING ADVERSE EFFECTS ASSOCIATED WITH TRANSFUSION OF AGED RED BLOOD CELLS

(76) Inventors: Steven L. Spitalnik, New York, NY (US); Eldad A. Hod, Tenafly, NY (US); Gary M. Brittenham, New York, NY (US)

(21) Appl. No.: 13/309,040

(22) Filed: Dec. 1, 2011

Related U.S. Application Data
(63) Continuation-in-part of application No. PCT/US2010/001610, filed on Jun. 1, 2010.
(60) Provisional application No. 61/187,600, filed on Jun. 16, 2009.
(60) Provisional application No. 61/275,579, filed on Aug. 31, 2009.

(51) Int. Cl. A61K 38/02; B01J 19/00; A61K 38/43; A61K 31/765; A61K 31/715; A61K 31/555; A61K 31/16; A61K 31/4439; A61K 31/44; A61K 31/444; A61K 31/15; A61K 31/175; A61K 31/4196; A61K 31/47; A61P 7/00; C12N 5/078

(52) U.S. Cl. 424/78.37; 435/2; 422/44; 514/5.4; 424/94.1; 514/54; 514/184; 514/616; 514/342; 514/348; 514/332; 514/639; 514/582; 514/383; 514/311

(57) ABSTRACT

The present invention provides, inter alia, methods for ameliorating an adverse effect in a patient caused by an acute transfusion into the patient of a composition containing aged red blood cells using an iron chelator. Apparatuses and kits for ameliorating such adverse effects are also provided.

### CROSS-REFERENCE TO RELATED APPLICATIONS

This application is a continuation-in-part application of International Application No. PCT/US2010/001610, filed Jun. 1, 2010, which claims benefit to U.S. Provisional Patent Application Ser. No. 61/187,600, filed Jun. 16, 2009 and U.S. Provisional Patent Application Ser. No. 61/275,579, filed Aug. 31, 2009. The entire contents of all of the above applications are hereby incorporated by reference as if recited in full herein.

### GOVERNMENT FUNDING

This invention was made with government support under grant numbers R01-HL098014 and K08-HL103756 awarded by the National Institutes of Health (NIH). The government has certain rights in the invention.

### FIELD OF THE INVENTION

The present invention is directed, inter alia, to methods, kits, and compositions for ameliorating the adverse effects associated with acute transfusion of aged red blood cells using iron chelators.

### BACKGROUND OF THE INVENTION

Approximately 14 million units of packed red blood cells (RBCS) are transfused in the United States every year [1]. In addition, intensive care units (ICUs) comprise 10% of all hospital beds, 4.4 million Americans are admitted to ICUs every year [2], and 44% of ICU patients are transfused with at least one RBC unit during their stay (mean of 4.6 units) [3]. The mean storage time of these units before transfusion is 17 days [1]. Several observational studies [4-9] and a large, randomized, prospective trial [10] suggest that transfusion per se increases morbidity and mortality in critically ill patients, an effect that increases with RBC storage time. In observational studies, increases in mortality [7, 11-13], serious infections [7, 12, 14], multi-organ failure [12, 15], and length of stay [7, 13] correlated with transfusion of older stored RBCs in critically-ill patients. Recently, a retrospective study of about 6000 cardiac surgery patients identified significantly higher rates of in-house mortality, sepsis/septicemia, and one year mortality in patients transfused with RBCs stored for greater than vs. less than 14 days [12]. Although controversial [16], these studies raise fundamental questions about the efficacy of transfusion with older stored RBCs; in addition, the mechanisms responsible for increased morbidity and mortality remain largely unknown [17].

Although >70% of RBC units transfused in the United States are leukoreduced (i.e. filtered to achieve a 3-log₁₀ leukocyte reduction) [1], most studies documenting the adverse effects of transfusion used non-leukoreduced RBCs. Although the mechanism(s) is/are still unclear, some adverse effects may be due to contaminating leukocytes in the RBC unit [18]. However, a significantly greater number of patients in the “older” stored RBC group in the recent study by Koch et al. [12] received leukoreduced RBCs. In addition, in a recent study of trauma patients who only received leukoreduced RBCs, transfusion of older stored units was associated with increased mortality, renal failure, and pneumonia [19]. Thus, leukoreduction does not eliminate the adverse effects of stored RBCs, although it may lessen their severity.

The biochemical and biomechanical changes occurring during storage in vitro, which reduce RBC function and survival, are collectively known as the “RBC storage lesion” [17]. These include ATP depletion [20], 2,3-diphosphoglycerate depletion [21], membrane vesiculation [22], protein and lipid oxidation [23, 24], decreased S-nitrosohemoglobin [25], decreased surface sialylation [26], decreased CD47 expression [27], increased phosphatidylserine exposure [28], and decreased deformability [29]. Some of these are exacerbated when leukocytes are present during storage [30]. In addition, RBC damage induced by increased storage time leads to increased levels of non-transferrin-bound iron in the supernatant [31].

Therefore, the Food and Drug Administration (FDA) mandates that the maximal allowable shelf life of stored RBCs requires maintenance of cellular integrity (i.e. free hemoglobin must be <1% of total hemoglobin in an RBC unit) and adequate 24-hour RBC survival post-transfusion (i.e. ≧75%); however, these are surrogate markers of therapeutic benefit [17]. Depending on the preservative, the maximal human RBC storage period is 35-42 days. Although the storage lesion is complex, and uncertainty remains regarding the mechanism(s) responsible for reduced RBC viability post-transfusion, the end result is decreasing 24-hour survival of transfused RBCs with increasing storage time. Despite the FDA requirement that, at outdate, on average, ≧75% of transfused RBCs must survive for 24 hours, the standard deviation in most studies is large and problematic [32]. Indeed, 24-hour survival is <75% for many transfusions [32, 33]. In addition, most RBC clearance occurs within the first hour post-transfusion [33]. One human RBC unit contains 220 to 250 mg of iron. Thus, rapid RBC clearance of up to 25% of even a single unit acutely delivers a substantial load of hemoglobin iron to the monocyte-macrophage system. Finally, although RBC survival studies for FDA licensure are typically performed in healthy volunteers, 24-hour post-transfusion RBC survival is even lower in critically-ill patients [33, 34]. Thus, this has led some to argue that more emphasis should be placed on studying the approximately 25% of RBCs that are cleared as a possible cause of impaired host defenses [30].

The net effect of the RBC storage lesion in vitro is rapid clearance in vivo. Although transfusion of some RBC units may result in >25% clearance, one may assume that 25% of the RBCS are cleared at the FDA-allowable outdate. Because an average unit contains about 1.5×10¹² RBCs, this implies that about 4×10¹¹ nonviable RBCs are handled by the approximately 10¹¹ phagocytes in the monocyte-macrophage system [30]. Therefore, in this setting, the total iron load delivered to the monocyte-macrophage system is approximately 60 mg per unit of stored RBCs in as little as a 1-hour time span [33]. To put this into perspective, in a healthy adult at steady state, about 25 mg of iron (derived from about 25 ml of senescent RBCs) is cleared daily by the monocyte-macrophage system (i.e. about 1 mg/hour). Thus, transfusion of an older unit of stored RBCs can acutely deliver up to a 60-fold increase in the hourly “dose” of iron. Even transfusions of relatively fresh RBCs, which may have about 90% 24-hour survival, can acutely deliver a significant load of iron, and many patients receive multiple RBC units.

Both mouse and human studies support the concept that increased intracellular iron in macrophages increases the pro-inflammatory cytokine response to various stimuli. The mechanism by which iron enhances inflammatory responses is thought to be due to increased production of reactive oxygen species, stemming from iron's involvement in the Fenton reaction, which catalyzes conversion of H₂O₂ to the hydroxyl radical, a potent oxidizing agent [35-37]. This altered redox environment induces the synthesis and secretion of pro-inflammatory cytokines, including tumor necrosis factor (TNF)-α, interleukin(IL)-6, macrophage inflammatory protein (MIP)-1, and monocyte chemoattractant protein (MCP)-1 [35, 36, 38, 39]. For example, in hereditary hemochromatosis, a human disorder of iron metabolism, macrophage iron levels are decreased. Similarly, macrophages in mouse hemochromatosis models have less intracellular iron; interestingly, Salmonella-induced intestinal inflammation is attenuated in these mice [40] and TNF-α and IL-6 expression are decreased in response to Salmonella or lipopolysaccharide (LPS). This effect of decreased intracellular iron inhibiting cytokine production was reproduced using cell-permeable iron chelators in wild-type macrophages [40]. In contrast, in a mouse model of alcoholic liver disease [41], Kupffer cells had increased iron and increased production of TNF-α and MIP-1; this was abolished by iron chelation, but enhanced by splenectomy. The latter increases hemoglobin iron delivery to Kupffer cells, supporting the role of iron priming in pro-inflammatory cytokine expression. In humans, decreasing or increasing intracellular iron also decreases or increases pro-inflammatory cytokine responses to relevant stimuli, respectively [42]. For example, monocytes from patients with hereditary hemochromatosis produced less TNF-α in response to LPS, as compared to healthy controls or patients with iron-loading anemias [42]. As presently understood, no human studies examining the effect of older stored RBC transfusions on cytokines have been reported. Nonetheless, higher IL-6 and IL-8 levels were seen post-transfusion in surgery patients [43-45]; cytokine levels were higher in those receiving autologous, rather than allogeneic RBCS, suggesting that this response was not due to an alloimmune mechanism [42].

In the systemic inflammatory response syndrome, an over-exuberant inflammatory response can lead to septic shock and multiple organ dysfunction, which causes significant morbidity and mortality [46]. Macrophages are central to this phenomenon, producing cytokines that initiate, perpetuate, and modulate this response [46]. The fate of uncommitted macrophages is dictated by cytokines [47]. Although differentiation down the classical pathway depends on interferon (IFN)-γ-dependent activation by T helper 1-type responses, alternative pathway activation depends on IL-4 and IL-13, which are T helper 2-type cytokines [48, 49]. Alternatively-activated macrophages do not kill intracellular pathogens and may play a role in the compensatory anti-inflammatory response syndrome [49], which regulates the systemic inflammatory response syndrome [50]. For example, mice with severe systemic inflammatory response syndrome (e.g. from acute pancreatitis) are susceptible to infections [51], which is partly due to alternatively-activated macrophages generated from resident macrophages by MCP-1 [50]. Because they control the intensity of effector responses, T cells play a major regulatory role in inflammation; for example, regulatory T cells are important in secondary infections and autoimmune responses [52]. Interestingly, the incidence of secondary infections is linked to transfusion of older stored RBCs [12] and RBC autoantibody formation is linked to transfusion [53]. Thus, the pro-inflammatory insult caused by transfusion of older stored RBCs may alter T cell subsets as a compensatory anti-inflammatory response. Regulatory T cells can also induce alternatively-activated macrophages [54], thus providing another pathway for impairing host defenses.

Extracellular non-transferrin-bound iron delivered by transfusion of older stored RBCs may also be pathologically relevant. Alternatively, non-transferrin-bound iron may “spill over” into the plasma following RBC phagocytosis if the monocyte-macrophage system is acutely overwhelmed by the need to handle massive amounts of iron.

When plasman iron is not sequestered by transferrin, the non-transferrin-bound iron can participate in redox reactions leading to oxidative damage, cytotoxicity, and enhanced expression of adhesion molecules [55, 56]. For example, in humans, elevated plasma non-transferrin-bound iron levels in vivo correlated with elevated soluble intercellular adhesion molecule (ICAM)-1 levels (a marker of activated endothelial cells) [57]. In addition, when human umbilical vein endothelial cells and monocytes were incubated in vitro with medium containing non-transferrin-bound iron, there was increased intercellular adhesion, increased endothelial cell expression of vascular cell adhesion molecule (VCAM)-1, ICAM-1, and E-selectin, and increased monocyte expression of integrin α4β1 (the ligand of VCAM-1) and integrin αLβ2 (the ligand for ICAM-1). Interestingly, these adhesive effects were blocked by cell-permeable iron chelators and anti-oxidants. Similarly the pro-oxidant effects induced in vitro by elevated non-transferrin-bound iron levels in plasma from β-thalassemia patients were rapidly inhibited (within 30 minutes) by treating the patients in vivo with an iron chelator [58].

Sickle cell disease is an important medical problem in the United States and many of its complications, such as stroke, are ascribed to increased intercellular adhesion between various cell types in the circulation (e.g. RBCs, endothelial cells, platelets, and leukocytes); in addition, pro-inflammatory cytokines may be important in this process [61]. Chronic RBC transfusions are effective in preventing these major complications [62]. However, despite their efficacy, there are no evidence-based standards of practice for RBC transfusion in sickle cell disease with regard to RBC storage time, washing, and/or cryopreservation [63]. Thus, it is important to consider the role RBC transfusions may play in producing increased levels of pro-inflammatory cytokines.

Although some patients, particularly children, can be managed by simple transfusions every 2-6 weeks, it is recommended that they only be transfused to a hematocrit of about 30%, otherwise complications may occur due to increased viscosity [64, 65]. In this setting, hemoglobin S levels remain at 10-20% and the patients exhibit low levels of ongoing hemolysis (e.g., evidenced by elevated reticulocyte counts). Thus, despite a substantially improved prognosis, they remain in a chronic hemolytic state. In addition, sickle cell disease has the hallmarks of a chronic inflammatory disorder, presumably due to ongoing hypoxia-reperfusion injury [66-69]. Therefore, although sickle cell disease patients can have elevated levels of cytokines, such as IL-6 [70, 71], their chronic underlying hemolysis and continuous pro-inflammatory state can, paradoxically, be associated with normal levels of other inflammatory mediators, perhaps because of up-regulation of compensatory mechanisms, such as heme oxygenase-1 [67, 71]. These phenomena may offer some relative protection against subsequent iron-mediated insults. Such “relative protection” may be analogous to the finding that prior exposure of mice to LPS induces “tolerance” to subsequent LPS exposure [72, 73].

As with sickle cell disease, β-thalassemia patients also benefit from chronic transfusion therapy [74]. In addition, they may show signs of a chronic inflammatory state [75], although they usually have lower levels of circulating pro-inflammatory cytokines [71, 76]. This may reflect lower levels of ongoing hemolysis in chronically transfused individuals and/or relate to the underlying pathophysiology of the disease, such as increased ineffective erythropoiesis.

Because of multiple RBC transfusions, these patients suffer from parenchymal iron overload leading to eventual cardiac, hepatic, and endocrine dysfunction [74]. Therefore, they are treated with chelators to prevent chronic parenchymal iron overload. Iron chelation may also modulate circulating non-transferrin-bound iron levels in these patients following RBC transfusion [77, 78], although this has not been studied in great detail. Indeed, it is interesting that circulating non-transferrin-bound iron levels remain elevated in some chronically transfused sickle cell disease and β-thalassemia patients despite ongoing chelation therapy [71, 79]; RBC transfusion may further increase non-transferrin-bound iron in these disease settings.

Because the supernatant of packed RBC units may contain biologically-active constituents that lead to adverse outcomes post-transfusion, such as allergic and febrile transfusion reactions (particularly in the non-leukoreduced setting), some believe that washed RBCs provide a superior product for treating patients with hemoglobinopathies [63]. Although washed RBC units are cumbersome for blood banks to provide, because of the labor involved and their short 24-hour outdate, newer methods using closed systems allow washed RBCs to be stored for significantly longer periods of time [80]. However, in the latter case, the parameters of RBC quality in vitro deteriorate with increasing storage after the washing step [80], suggesting that 24-hour RBC survival may also be affected.

Chronically transfused hemoglobinopathy patients may become alloimmunized to multiple blood group antigens, although with prospective phenotype matching this occurs less frequently than in the past [81]. Nonetheless, in this setting, patients may require transfusion with difficult to obtain, rare units. As a result, blood centers stock multiple cryopreserved RBC units with particular antigen phenotypes, which can then be used to transfuse these patients [63]. However, cryopreserved RBCs may have less than optimal 24-hour survival post-transfusion, particularly if they are frozen after significant storage times in vitro at 4° C. or stored post-thaw in vitro at 4° C. for significant lengths of time [27, 82-84]. Nonetheless, deglycerolizing cryopreserved RBC units involves extensive washing, which may ameliorate the adverse effects of transfusion due to substances in the supernatant, similar to what was described above regarding washed RBCs.

In view of the foregoing, it would be advantageous to provide methods, kits, and compositions for ameliorating the disadvantages noted above with respect to using aged RBCs in blood transfusions, particularly acute blood transfusions. The present invention is directed to providing, inter alia, such methods, kits, and compositions.

### SUMMARY OF THE INVENTION

One embodiment of the present invention is an apparatus for ameliorating an adverse effect in a patient caused by an acute transfusion into the patient of a composition comprising aged red blood cells. The apparatus comprises an inner surface that is in sterile contact with the composition and an effective amount of an iron chelator.

Another embodiment of the present invention is a kit for ameliorating an adverse effect in a patient caused by an acute transfusion into the patient of a composition comprising aged red blood cells. The kit comprises a container comprising an effective amount of an iron chelator packaged together with instructions on how to administer the iron chelator to the composition directly, to a blood product-related apparatus, or to a patient in need thereof.

A further embodiment of the present invention is a method for ameliorating an adverse effect in a patient caused by an acute transfusion into the patient of a composition comprising aged red blood cells. This method comprises providing an iron chelator, which is capable of chelating iron released by macrophage phagocytosis of the aged red blood cells, wherein the chelator ameliorates the adverse effect in the patient.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a proposed mechanism according to the present invention for the effects of transfusion of stored RBCs.

FIG. 2 shows the survival of fresh and stored RBCs. Briefly, C57BL/6 mice were transfused with 51 Cr-labeled fresh (triangles), 2-week old (squares; FIG. 2A), or 3-week old (squares; FIG. 2B) leukoreduced RBCs stored in citrate-phosphate-dextrose-adenine (CPDA-1, 100 μL at 50% hematocrit; 3-5 mice/group). Retro-orbital blood was collected in microhematocrit tubes immediately, and at 1, 2, and 24 hours post-transfusion; these were centrifuged and the height of the packed RBC column measured. Survival was calculated as 100 multiplied by the ratio of counts per minute per mm of RBC column height at each time point versus the immediate time point.

FIG. 3 shows dose-responsive increases in plasma pro-inflammatory cytokine levels after transfusion of stored RBCs. Briefly, C57BL/6 mice were transfused with fresh or 2-week old leukoreduced RBCs stored in CPDA-1 at a low (i.e. 200 μL: “1 unit”) or high (400 μL: “2 units”) dose. 200 μL was determined to be the mouse equivalent to 1 unit of human packed RBCs, based on the assumption that a 25 g mouse has a 2 mL blood volume and RBCs were transfused at a 50% hematocrit. Mice were exsanguinated 2 hours post-transfusion and plasma cytokine levels were measured using a multiplex flow cytometry assay (Flex kit, BD). Cytokine levels are indicated (±SEM) and the conditions are indicated below the panels (MCP-1 (left panel); IL-6 (right panel)). * indicates p<0.05 compared to untreated mice. ** indicates p<0.05 compared to untreated mice and compared to low dose stored RBC transfusion-treated mice.

FIG. 4 shows erythrophagocytosis by Kupffer cells in vivo. Briefly, 057BI/6 mice were transfused with 3-week stored or fresh RBCs. Necropsies were performed 2 hours post-transfusion; sections of liver were stained with hematoxylin and eosin and then examined by light microscopy. In a representative image from a mouse transfused with stored RBCs, the arrow identifies a Kupffer cell with about 4 ingested RBCs.

FIG. 5 shows that total iron is significantly increased in the liver, spleen, and kidney of mice transfused with stored RBCs. Briefly, C57BL/6 mice were transfused with fresh (400 μL—white bar) or 2-week old RBCs (gray bar) stored in CPDA-1 (400 μL at 50% hematocrit; 13 mice per group). Mice were sacrificed 2 hours post-transfusion and total iron was measured in the liver, spleen, and left kidney using a wet ashing procedure. Bars indicate the total iron increase as compared to control non-transfused mice (* represents p<0.05 in a 2-tailed Student's t-test comparing transfusion of fresh and stored RBCs).

FIG. 6 shows that the pro-inflammatory response requires transfusion of intact stored RBCs. Briefly, C57BL/6 mice were transfused with 2-week stored RBCs (Stored; 400 μL), 2-week stored RBCs washed 3 times in 10 volumes of normal saline (Pellet; 400 μL), supernatant (400 μL), or RBC ghosts (400 μL) derived from 2-week stored RBCs. Mice were sacrificed 2 hours post-transfusion and plasma cytokine levels were measured using a multiplex flow cytometry assay (MCP-1 (left panel); IL-6 (right panel)). The mean cytokine levels are indicated (±SEM) and the conditions are denoted below the panels. * indicates p<0.05 compared to mice transfused with 2-week stored RBCs.

FIG. 7 shows that transfusion of stored RBCs increases non-transferrin-bound iron. Briefly, C57BL/6 mice were transfused with 2-week stored RBCs (Stored; 400 μL), 2-week stored RBCs washed 3 times in 10 volumes of normal saline (Pellet; 400 μL), or supernatant (400 μL). Mice were sacrificed 2 hours post-transfusion and plasma non-transferrin-bound iron was measured as described [93]. The mean plasma non-transferrin-bound iron levels are indicated (±SEM) and the conditions are denoted below the panels. * indicates p<0.05 compared to untransfused mice, which all had undetectable levels (n=5; not shown). Note: transfusion of supernatant resulted in no detectable non-transferrin-bound iron at 2 hours post-transfusion.

FIG. 8 shows that LPS and transfused stored RBCs synergize to exacerbate and prolong the cytokine storm. Briefly, mice were injected with LPS (100 μg/mouse of E. coli 0111:B4; Sigma, St. Louis, Mo.) alone, 400 μL of 2-week stored RBCs alone, or concomitantly with LPS (100 μg/mouse) and 400 μL of either fresh RBCs, 2-week stored RBCs, or RBC ghosts derived from stored blood. Mice (5-10/group) were sacrificed 24 hours post-treatment and cytokine levels were quantified (MCP-1 (left panel); IL-6 (right panel)). Cytokine measurements are indicated (mean±SEM) and the conditions are denoted below the panels. * indicates p<0.05 compared to mice transfused with fresh RBCs.

FIG. 9 shows that iron chelation inhibits the pro-inflammatory cytokine response in mice transfused with older, stored RBCs. Briefly, C57BI/6 mice were transfused with 400 μL of 2-week stored RBCs alone (n=13) or after pretreatment with either 120 mg/kg intravenous deferoxamine (DFO) 5-10 minute pre-transfusion (n=10) or 30 mg/kg deferasirox (Exjade), administered by oral gavage 24 and 6 hours pre-transfusion (n=6). Plasma cytokine levels were quantified at 2 hours post-transfusion. Fresh RBCs (400 μL) were transfused as a control (n=13). Cytokine levels (MCP-1 (top panel); IL-6 (middle panel); KC (CXCL1) (bottom panel)) are indicated (mean±SEM) and the conditions are denoted below the panels. * indicates p<0.05 compared to mice transfused with stored RBCs. All transfusions with stored RBCs and chelators had significantly elevated cytokine levels as compared to fresh RBC transfusions (p<0.05).

FIG. 10 shows the timing of autologous RBC donations, transfusions, and blood samples during a study of healthy volunteers. Participation will involve 45 days from first donation to final phlebotomy. Blood draws will occur prior to each transfusion and at 0, 1, 2, 4, 24, and 72 hours post-transfusion.

FIG. 11 shows a representative study outline according to the present invention for one patient. Each vertical line on the timeline represents one month. Above the timeline represents dedicated donor participation, below the timeline represents recipient participation. Six transfusions are proposed per patient, encompassing 3 paired transfusion events.

FIG. 12 shows another representative study outline according to the present invention. Each vertical line on the proposed timeline represents one month. Above the timeline represents dedicated donor participation; below the timeline represents recipient participation. Two transfusions per patient are proposed, representing the fourth paired transfusion event.

FIG. 13 shows that transfusions of stored RBCs lead to increased RBC clearance, tissue iron delivery, and circulating non-transferrin bound iron (NTBI) levels, as compared to transfusions of fresh RBCs, stored RBC-derived supernatant, or ghosts prepared from stored RBCs. All transfusion recipients were male C57BL/6 mice (8-12 weeks old). The results are presented as mean±standard error of the mean (s.e.m.) except where specified. FIG. 13A shows the results from one representative experiment conducted as follows: Leukoreduced fresh FVB/NJ mouse RBCs (<24 hours storage; n=3) and stored RBCs (2-week storage; n=5) were transfused (400 μL at 17.0-17.5 g/dL of hemoglobin) and survival of transfused RBCs was calculated by dual-label flow cytometric tracking at 10 minutes, 30 minutes, 1-hour, 2-hours (only for stored RBCs), and at 24-hours post-transfusion. *P=0.04. FIG. 13B shows a representative image of spleens obtained from mice 2-hours after transfusion with fresh RBCs or stored RBCs. FIG. 13C shows the mean spleen weights of mice transfused with fresh RBCs (n=13) and stored RBCs (n=13). *P=0.02. To obtain the results shown in FIG. 13D, aliquots (400 μL) of fresh RBCs (n=13), stored RBCs (n=13), washed stored RBCs (n=13), stored RBC-derived supernatant (SN; n=12), and ghosts prepared from stored RBCs (n=8) were transfused. Total iron was measured in organs obtained at necropsy 2-hours post-transfusion; the increases in iron are shown as compared to those measured in control, untransfused mice (n=12). The results are combined from three separate experiments. Liver (left panel): *P=0.04, **P=0.0002, ***P=0.03; spleen (middle panel): *P<0.0001; and kidney (right panel): *P=0.002, **P=0.0004; as compared to fresh RBC transfusions. To obtain the results shown in FIG. 13E, mice were transfused as labeled (n=5 per group) and plasma NTBI was measured 2-hour post-transfusion. Note: absent error bars indicate undetectable NTBI levels. The results are representative of two separate experiments. *P=0.008, **P=0.01 as compared to fresh RBC transfusions.

FIG. 14 shows that transfusions of stored RBCs induce dose-responsive pro-inflammatory responses in mice. To obtain the results shown in FIG. 14A, untransfused C57BL/6 mice (n=13) or mice transfused with fresh RBCs (<24-hour storage; 1u (i.e. 1 unit)=200 μL, n=5; 2u=400 μL, n=17), stored RBCs (2-week storage; 1u=200 μL, n=5; 2u=400 μL, n=17), washed stored RBCs (400 μL; n=13), stored RBC-derived supernatant (SN; 400 μL; n=12), and ghosts prepared from stored RBCs (400 μL; n=8) were sacrificed 2-hours post-transfusion and plasma cytokine levels measured (IL-6 (top panel) and MCP-1 (bottom panel) are shown). *P<0.0001, **P=0.003 as compared to equivalent doses of transfused fresh RBCs. Results shown in 14B are representative of two experiments. SAA1-luciferase reporter mice were transfused with 200 μL of either fresh RBCs (<24-hours storage) or stored RBCs (2-weeks storage) and luciferase activity measured by noninvasive bioluminescence imaging at multiple times up to 24-hours post-transfusion (n=3 per group). To obtain the results shown in FIG. 14C, bioluminescence was quantified over the hepatosplenic region of SAA1-luciferase reporter mice transfused with fresh RBCs (n=6; gray circles) or stored RBCs (n=6; black squares). *P=0.002. FIG. 14D shows that circulating SAA1 protein levels in SAA1-luciferase reporter mice 24-hours after transfusion with fresh RBCs or stored RBCs (n=6 per group). *P=0.002. Results are combined from two separate experiments.

FIG. 15 shows that transfusion of stored RBCs synergizes with the inflammatory response to LPS and enhances bacterial growth. Results shown in FIG. 15A are representative of two experiments. C57BL/6 mice were infused with a sub-clinical dose of LPS (E. coli 0111:B4; 30 μg per mouse by tail vein injection) followed by transfusion of 400 μL of fresh RBCs or stored RBCs. Mice were sacrificed at 24-hour post-transfusion and plasma cytokines measured (n=5 per group). *P=0.008, **P=0.003 as compared to mice infused LPS+ stored RBCs. To obtain the results shown in FIG. 15B, plasma (100 μL) was obtained from mice 2-hours post-transfusion with 400 μL of fresh RBCs (n=15), stored RBCs (n=24), stored RBC-derived supernatant (n=12), washed stored RBCs (n=13), or ghosts prepared from stored RBCs (n=8). Plasma was also obtained from control untransfused mice (n=14) or 24-hours post-transfusion with stored RBCs (n=8). Samples were incubated at 37° C. with shaking with about 1×10⁶ CFU of E. coli, as labeled. Bacterial growth was monitored every 30 minutes by absorbance at 600 nm for up to 5 hours. Bacterial growth in plasma from mice 2-hours post-transfusion with stored RBCs or washed stored RBCs began diverging from all other groups at 2.5-hour of incubation in vitro, and area under the curve (AUC) (in parentheses) for each group was significantly different as indicated. To obtain the results shown in FIG. 15C, pooled plasma samples (100 μL) from mice 2 hours after transfusion with 400 μL of fresh RBCs or stored RBCs were supplemented with either ferric citrate (20 μM), sodium citrate (20 μM), bovine serum albumin (BSA; 80 μM), or protoporphyrin IX (20 μM), and then incubated at 37° C. with shaking with ˜1×10⁶ CFU of E. coli. Bacterial growth was monitored every 30 minutes by absorbance at 600 nm for up to 5 hours in replicates of 5 per group. Area under the curve (AUC) (in parentheses) for growth in plasma from mice transfused with fresh RBCs, supplemented with or without sodium citrate, BSA, or protoporphyrin IX, differed significantly from the other 3 groups. To obtain the results shown in FIG. 15D, pooled plasma (n=4) were incubated with the iron chelator, DFO (20 μM), or with the iron-chelated form of DFO, ferroxamine (FO) (20 μM) and inoculated with E. coli as shown for the previous experiment. The AUC (in parentheses) for growth in plasma with DFO significantly differed from all other groups. To obtain the results shown in FIG. 15E, pooled plasma (n=5) was incubated with the iron chelator, 2,2′-dipyridyl (400 μM), with or without ferric citrate (133 μM) and inoculated with E. coli, as shown for the previous experiment. The AUC (in parentheses) for growth in plasma with 2,2′-dipyridyl significantly differed from all other groups; *P<0.05. Results are representative of at least 2 experiments and are shown as mean (±SEM). Note that the absence of an error bar is indicative of highly reproducible replicates with pooled plasma.

FIG. 16 shows that DFO treatment decreases the pro-inflammatory response induced by stored RBC transfusions. To obtain the results shown in FIG. 16A, mice were pretreated with a PBS vehicle control (n=28) or with 3 mg of DFO, with (n=15) or without (n=31) the addition of equimolar ferric citrate, immediately before transfusion with stored RBCs (400 μL). Mice were sacrificed 2 hours after transfusion, and plasma cytokine levels were measured; *P<0.05; **P<0.01; ***P<0.001 compared with mice infused with PBS vehicle and transfused stored RBCs. To obtain the results shown in FIG. 16B, bioluminescence was quantified for 24 hours after transfusion over the hepatosplenic region of SAA1-luciferase reporter mice transfused with 200 μL of fresh RBCs (n=3; •), the PBS vehicle control and stored RBCs (n=3; ▪), or 3 mg of DFO and stored RBCs (n=6; ▴); P=0.095 at 4 and 6 hours after transfusion comparing vehicle-treated and DFO-treated mice. FIG. 16C shows the proposed mechanistic pathway (the “iron hypothesis”) explaining how transfusion of older stored RBCs may induce adverse effects in patients. Transfusion of stored, but not fresh, RBCs delivers an acute bolus of RBCs and RBC-derived iron to the monocyte/macrophage system resulting in oxidative stress and inflammatory cytokine secretion. Some of the macrophage-ingested iron is also released back into the circulation (i.e., NTBI) where it can also cause oxidative damage and enhance bacterial proliferation. SIRS indicates systemic inflammatory response syndrome.

FIG. 17 shows that transfusions of stored RBCs induce dose-responsive pro-inflammatory responses. Untransfused C57BL/6 mice (n=13) or mice transfused with fresh RBCs (<24-hour storage; 1u=200 μL, n=5; [i.e., 1 human equivalent unit=200 μL); 2u=400 μL, n=17), stored RBCs (2-week storage; 1u=200 μL, n=5; 2u=400 μL, n=17), washed stored RBCs (400 μL; n=13), stored RBC-derived supernatant (SN; 400 μL; n=12), ghosts prepared from stored RBCs (400 μL; n=8), and stroma-free lysate derived from stored RBCs (400 μL, n=8) were sacrificed 2-hours post-transfusion and plasma cytokine/chemokine levels measured (as labeled); *P<0.05; **P<0.01; ***P<0.001 compared with fresh RBCs.

FIG. 18 shows that transfusions of stored RBCs synergize with the inflammatory response to LPS. C57BL/6 mice were infused with a sub-clinical dose of LPS (E. coli 0111:B4; 30 μg per mouse by tail vein injection) followed by transfusion with 400 μL of either fresh RBCs or stored RBCs. Mice were sacrificed at 24-hour post-transfusion (or earlier if moribund) and plasma cytokines/chemokines measured (one representative experiment of two, n=5 per group). *P=0.008, **P=0.003, ***P=0.03, ****P=0.01, *****P=0.004 as compared to mice infused LPS+ stored RBCs.

FIG. 19 shows that DFO treatment inhibits the pro-inflammatory response induced by stored RBC transfusions. Mice were untreated or pretreated with 3 mg of DFO immediately before stored RBC transfusion (400 μL; n=14 per group). Mice were sacrificed 2-hours post-transfusion and plasma cytokine/chemokine levels measured. Boxes represent 25^{th} to 75^{th} percentile with median line. Whiskers represent range. KC: P=0.07; MIP-1β: P=0.18; TNF-α: P=0.097.

FIG. 20 is a graph showing total bilirubin levels in serum, over time, in patients transfused with “fresh”, 3-day old RBC transfusions or the “old”, 42-day old RBC transfusions. Dotted horizontal lines represent the normal reference ranges.

FIG. 21 is a graph showing iron levels in serum, over time, in patients transfused with “fresh”, 3-day old RBC transfusions or the “old”, 42-day old RBC transfusions. Dotted horizontal lines represent the normal reference ranges.

FIG. 22 is a graph showing haptoglobin levels in serum, over time, in patients transfused with “fresh”, 3-day old RBC transfusions or the “old”, 42-day old RBC transfusions. Dotted horizontal lines represent the normal reference ranges.

FIG. 23 is a graph showing transferrin saturation in serum, over time, in patients transfused with “fresh”, 3-day old RBC transfusions or the “old”, 42-day old RBC transfusions. Dotted horizontal lines represent the normal reference ranges.

FIG. 24 is a graph showing NTBI levels in plasma, over time, in patients transfused with “fresh”, 3-day old RBC transfusions or the “old”, 42-day old RBC transfusions. Dotted horizontal lines represent the normal reference ranges.

FIG. 25 is a graph showing absolute neutrophil count in plasma, over time, in patients transfused with “fresh”, 3-day old RBC transfusions or the “old”, 42-day old RBC transfusions. Dotted horizontal lines represent the normal reference ranges.

FIG. 26 is a graph showing MCP-1 levels in plasma, over time, in patients transfused with “fresh”, 3-day old RBC transfusions or the “old”, 42-day old RBC transfusions.

FIG. 27 shows a perspective view of a representative apparatus according to the present invention.

FIG. 28 shows a cross-sectional view of the apparatus of FIG. 27, along the line A-A.

FIG. 29 shows a perspective view of an alternative embodiment of an apparatus according to the present invention.

FIG. 30 shows that macrophages are responsible for clearing transfused stored RBCs. All transfusion recipients and donors were syngeneic male C57BL/6 mice (8-12 weeks of age). The experimental conditions of the results shown in FIG. 30A were as follows. Mice were infused intraperitoneally with 2 mg of liposomal clodronate (n=9) or control PBS-liposomes (n=10) 48 hours before transfusion with stored RBCs. The 2-hour RBC survival was then measured. The 2-hour RBC survival (▪) is indicated for each mouse and the horizontal bar indicates the mean. The results are representative of 2 separate experiments; ***P=0.001 compared with treatment with PBS-liposomes. FIG. 30B shows representative images of histological sections of liver and spleen from mice treated with liposomal clodronate or control PBS-liposomes 48 hours before transfusion with stored RBCs, and stained with an anti-mouse F4/80 monoclonal antibody, as labeled. Note the absence of tissue macrophages in the liposomal clodronate-treated mice, as evidenced by the absence of brown staining cells. FIG. 30C shows representative images of histological sections from the liver of mice transfused with fresh or stored RBCs. Sections were stained with hematoxylin & eosin or with an anti-mouse F4/80 monoclonal antibody, as labeled. Arrows denote tissue macrophages that ingested RBCs. Brown staining is a result of F4/80 immunoreactivity of macrophages; the cytoplasmic staining is displaced to the periphery of the cells in mice transfused with stored RBCs because of the accumulation of ingested RBCs. Original magnification was 400. Typical representative examples derived from 5 necropsies are shown.

FIG. 31 shows that transfusion of stored RBCs induces dose-responsive proinflammatory cytokine responses. Hemoglobinemia, as detected by a prominent absorbance, was observed in all mice (n=8) transfused with stroma-free lysate derived from stored RBCs. Representative spectra of plasma (diluted 1:4 with PBS) obtained from mice 2 hours after transfusion with fresh RBCs (<24-hour storage), stored RBCs (2-week storage), or stroma-free lysate derived from stored RBCs are shown.

FIG. 32 shows that transfusions of older stored RBCs in humans raise circulating serum levels of interleukin-6 (FIG. 32A) and hepcidin (FIG. 32B). Circulating levels of analytes (as labeled) are shown for the “fresh”, 3-day old RBC transfusions and the “old”, 42-day old RBC transfusions (left and right graphs in each panel, respectively).

FIG. 33A shows a study design of healthy human volunteers.

FIG. 33B shows mean±SEM for hemoglobin levels from pre-transfusion to 72-hours after transfusion of either fresh or older red blood cells. The P value is as specified in the figure comparing the paired area under the curve of the mean hemoglobin levels for the N=14 volunteers from 0- to 24-hours after the fresh and older red blood cell transfusions. FIG. 33C shows the individual hemoglobin levels for each subject up to 24-hours post-transfusion. Vertical arrows denote pre-transfusion time points and horizontal dashed lines represent reference range values for men (blue) and women (pink).

FIG. 34 shows that potassium levels did not change and calcium levels decreased after transfusions of older red blood cells in healthy human volunteers. The mean±SEM for serum levels of potassium (FIG. 34A), total calcium (FIG. 34B), and corrected calcium calculated as ((0.8*(4.0−subject's albumin))+serum calcium) (FIG. 34C). The vertical arrow denotes the pre-transfusion time point and dotted lines represent the reference ranges. The P values are as specified in the figure comparing the paired area under the curve of the mean of the outcome parameter for the N=14 volunteers from 0- to 24-hours after the fresh and older red blood cell transfusions.

FIG. 35 shows that transfusions of older red blood cells resulted in laboratory values consistent with extravascular hemolysis in healthy volunteers. Mean±SEM for serum levels of total bilirubin (FIG. 35A) and conjugated bilirubin (FIG. 35B) from pre-transfusion to 72-hours after transfusion of both fresh and older red blood cells are shown. FIG. 35C shows the individual serum total bilirubin levels for all 14 volunteers from pre-transfusion to 72-hours after transfusion of both fresh and older red blood cells. FIG. 35D shows the mean±SEM for lactate dehydrogenase (LDH) and haptoglobin, from pre-transfusion to 72-hours after transfusion of both fresh and older red blood cells. Although iatrogenic hemolysis was induced during a difficult blood draw for two volunteers at 1-hour after the older red blood cell transfusion, these samples were still included in the analysis. Nonetheless, the analysis was not significantly altered by their exclusion. The vertical arrows in all panels denote the pre-transfusion time point and dotted lines represent the reference ranges (and in gray lines for LDH). The P values are as specified in the figure comparing the paired area under the curve of the mean of the outcome parameter for the N=14 volunteers from 0- to 24-hours after the fresh and older transfusions.

FIG. 36 shows that iron parameters and circulating non-transferrin-bound iron levels increased after transfusions of older red blood cells in healthy volunteers. The mean±SEM (FIG. 36A) and individual levels of serum iron (FIG. 36B); mean±SEM (FIG. 36C) and individual levels of transferrin saturation (FIG. 36D); increase in ferritin as compared to baseline levels (FIG. 36E) and increase in plasma non-transferrin-bound iron (FIG. 36F) are compared to baseline levels from pre-transfusion to 72-hours after transfusion of fresh and older red blood cells. Vertical arrows denote pre-transfusion time points and dotted lines represent the reference range (the reference range for change in ferritin and non-transferrin-bound iron is 0 by definition). The P values are as specified in the figure comparing the paired area under the curve of the mean of the outcome parameter for the N=14 volunteers from 0- to 24-hours after the fresh and older red blood cell transfusions.

FIG. 37 shows that serum levels of inflammatory markers did not increase after transfusions of older red blood cells as compared to fresh red blood cells in healthy volunteers. The mean±SEM for serum interleukin (IL)-6 levels (FIG. 37A), C-reactive protein (CRP) levels (FIG. 37B), and individual levels of CRP (FIG. 37C) from pre-transfusion to 72-hours after transfusion of fresh and older red blood cells are shown. Vertical arrows denote pretransfusion time points and dotted lines represent the reference range. The P values are as specified in the figure comparing the paired area under the curve of the mean of the outcome parameter for the N=14 volunteers (for IL-6) and N=12 (for CRP; the first two volunteers were not tested due to inadequate sample volume) from 0- to 24-hours after the fresh and older red blood cell transfusions.

FIG. 38 shows that sera obtained after transfusions of older red blood cells enhanced proliferation of a bacterial pathogen in vitro. FIG. 38A shows the bacterial growth of E. coli in serum samples obtained following fresh or older red blood cell transfusions, which was determined by serial optical density measurements at 600 nm for up to 5 hours after inoculation. Each point in the graph represents the mean±SEM of the area under the curve (AUC) of the resultant bacterial growth curve (N=14 paired values). FIG. 38B shows the relationship between the mean difference in bacterial growth between fresh and older red blood cell transfusions at each time point and the corresponding differences in plasma non-transferrin-bound iron levels using a Pearson correlation. The P values are as specified in the figure.

FIG. 39 shows the complete blood counts in healthy human volunteers after single unit transfusions of fresh or older red blood cells. The mean±SEM white blood cell (FIG. 39A), absolute neutrophil (FIG. 39B), and platelet counts (FIG. 390) from pretransfusion to 72-hours post-transfusion in volunteers transfused with either fresh or older red blood cells are shown. Vertical arrows denote pre-transfusion time points and horizontal dashed lines represent reference range values. The P value is as specified comparing the paired area under the curve of the means of the outcome parameters for the N=14 volunteers from 0 to 24 hours after the fresh and older transfusions.

FIG. 40 shows the basic metabolic parameters in healthy human volunteers after single unit transfusions of fresh or older red blood cells. The mean±SEM for serum sodium (FIG. 40A), chloride (FIG. 40B), bicarbonate (FIG. 40C), blood urea nitrogen (FIG. 40D), creatinine (FIG. 40E), and glucose (FIG. 40F) from pre-transfusion to 72 hours post-transfusion in volunteers transfused with either fresh or older red blood cells are shown. Vertical arrows denote pre-transfusion time points and horizontal dashed lines represent reference range values. The P value is as specified comparing the paired area under the curve of the means of the outcome parameters for the N=14 volunteers from 0 to 24 hours after the fresh and older transfusions.

FIG. 41 shows the liver function parameters in healthy human volunteers after single unit transfusions of fresh or older red blood cells. The mean±SEM for serum alanine aminotransferase (FIG. 41A), aspartate aminotransferase (FIG. 41B), total protein (FIG. 41C), albumin (FIG. 41D), and alkaline phosphatase (FIG. 41E) from pre-transfusion to 72-hours post-transfusion in volunteers transfused with either fresh or older red blood cells are shown. Vertical arrows denote pre-transfusion time points and horizontal dashed lines represent reference range values. The P value is as specified comparing the paired area under the curve of the means of the outcome parameters for the N=14 volunteers from 0 to 24 hours after the fresh and older transfusions.

### DETAILED DESCRIPTION OF THE INVENTION

One embodiment of the present invention is an apparatus for ameliorating an adverse effect in a patient caused by an acute transfusion into the patient of a composition comprising aged red blood cells. The apparatus comprises an inner surface that is in sterile contact with the composition and an effective amount of an iron chelator.

In the present invention, the apparatus may be any conventional device used in the storage or processing of compositions comprising aged red blood cells, such as, e.g., donated RBCs. For example, the apparatus may be a container for storing red blood cells for transfusion into a patient in need thereof such as, e.g., a conventional blood transfusion bag.

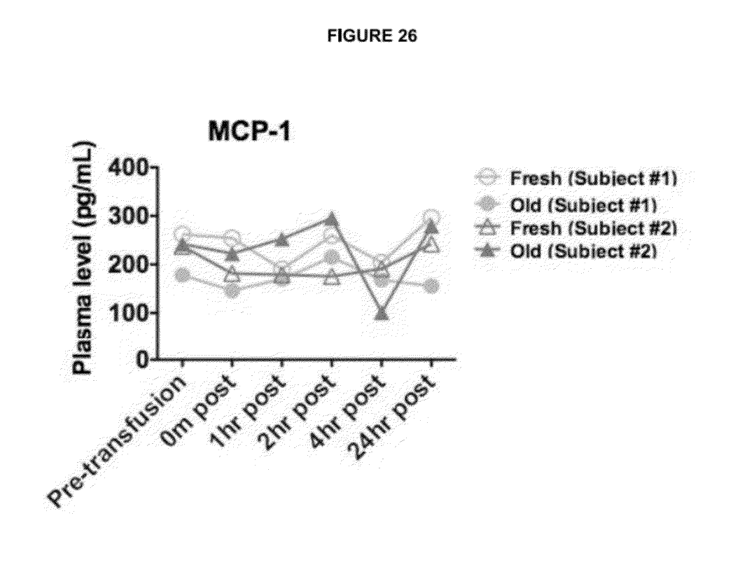

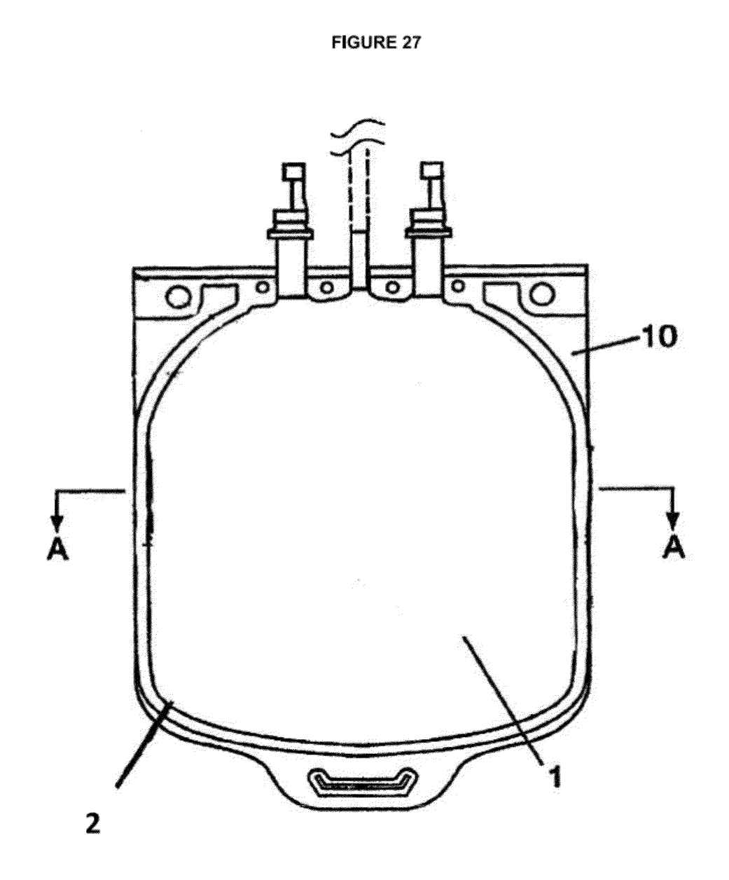

Turning now to FIG. 27, one aspect of this embodiment is a conventional blood bag. As shown, the apparatus 10 includes an inner surface 2, which defines an inner space 1. A cross-sectional view of the apparatus 10 along the line A-A is shown in FIG. 28. The inner surface 2′ is shown, which defines the inner space 1′ in which, e.g., the composition comprising RBCs is stored.

The iron chelator according to the present invention may be disposed on the inner surface 2′ of the apparatus 10′. In the present invention, the iron chelator may be applied, e.g., as a coating to the inner surface of the apparatus or may be impregnated into the inner surface using any appropriate conventional means. Other conventional methods for applying an iron chelator according to the present invention onto/into the inner surface of the apparatus are also contemplated.

Alternatively, the iron chelator of the present invention may be disposed within the inner space 1′ of the apparatus 10′ formed by the inner surface 2′ that is in sterile contact with the composition.

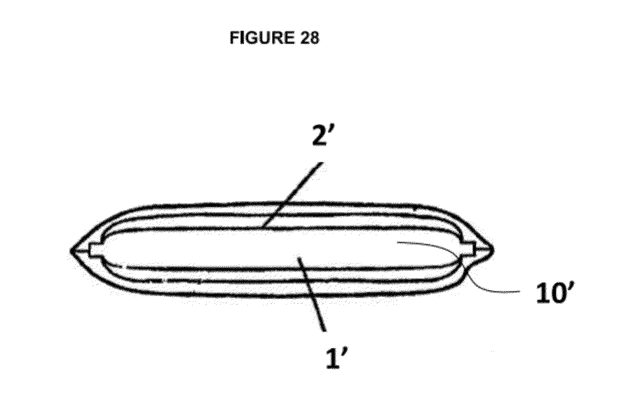

In yet another aspect of this embodiment, the apparatus may be any conventional blood filter. Turning now to FIG. 29, there is shown a representative blood filter 100 according to the present invention. The blood filter comprises an inner surface 21, and an inner space, 20. In the present invention, any conventional blood filter may be used. The iron chelator may be disposed on any inner surface of the blood filter that comes into contact with, e.g., RBCs. Thus, the chelator may be coated onto or impregnated into an inner surface 21 of the blood filter. Alternatively, the iron chelator may be disposed on the filter portion 22 of the blood filter. The location and means for disposing the iron chelator onto the blood transfusion bag or the blood filter is not critical so long as the iron chelator is brought into contact with the composition comprising aged red blood cells and is capable of chelating iron therefrom.

As used herein, an “acute” transfusion means a single transfusion, or a series of transfusions, that does not constitute part of a regimen of chronic transfusions performed in the course of treating a chronic medical condition in a subject. The term “chronic” transfusion includes single transfusions that are administered as part of a regular or frequent schedule of transfusions given to subjects with chronic medical conditions.

As used herein, “aged red blood cells” mean red blood cells (RBCs) that have been removed from a donor and stored outside the donor, typically in refrigerated storage, for a certain period of time such that they are no longer in optimal condition for use in transfusions. There may be some variability in the number of days of storage after which RBCs will be considered “aged” RBCs, dependent, for example, on the temperature at which the cells have been stored or the preservative(s) used. “Aged” RBCs include but is not limited to those RBCs considered to be “outdate” by the current FDA standards, such as those stored in refrigerated conditions for about 35-42 days. However, cells that have been stored outside of the body for less than about 35 days may also be considered to be not optimal for use in transfusion by those skilled in the art, and are considered “aged” for the purposes of the present invention. For example, in certain embodiments, the term “aged” RBCs may refer to cells that have been stored outside the donor for about 14 days or more, or about 16 days or more, or about 18 days or more, or about 20 days or more, or about 22 days or more, or about 24 days or more, or about 26 days or more, or about 28 days or more, or about 30 days or more, or about 32 days or more, or about 34 days or more. One of skill in the art will be able to determine whether such cells are considered aged, taking into account factors such as storage media, including preservative(s) used, storage temperature, percentage viability of RBCs, the percentage of cells that survive and circulate following transfusion into a subject (such as a test subject or a treatment subject), and certain biochemical or other test parameters, including, but not limited to, amount of pro-inflammatory cytokines, amount of transferrin-free iron, ATP depletion [20], 2,3-diphosphoglycerate depletion [21], membrane vesiculation [22], protein and lipid oxidation [23, 24], decreased S-nitrosohemoglobin [25], decreased surface sialylation [26], decreased CD47 expression [27], increased phosphatidylserine exposure [28], and decreased deformability [29], decreased corpuscular integrity, and/or a level of free hemoglobin greater than 1% of total hemoglobin, in the RBC sample.

As described above, there is some variability as to when RBCs are considered aged. Most frequently, a physician or other medical professional engaged in the blood donation and/or blood transfusion field will be able to make a determination regarding whether RBCs are considered aged.

As used herein, the term “iron chelator” means any substance capable of interacting with iron, including Fe(II) or Fe(III), that can prevent or interfere with adverse effects resulting from the acute transfusion. Non-limiting examples of iron chelators according to the present invention include apotransferrin, lactotransferrin, metalloenzymes, an hydroxamic acid polymer (including those disclosed by Varaprased et al. [110]), a phosphorylated myo-inositol polymer (including those disclosed by Lemma et al. [111]), heme B, heme A, heme C, desferoxamine (DFO), desferrithiocin (DFT), desferri-exochelin (D-Exo), (S)-DMFT, (S)-DADMDFT, (S)-DADFT, 4′-(OH)-DADFT, 4′-(OH)-DADMDFT or its hexadentate derivative BDU [112], deferiprone (L1), an hydroxypyridinone ester prodrug whose metabolism yields a hydroxypyridinone analog, CP94, CP502, CP365, CP102, CP41, CP38, LiNAII, Pr-(Me-3,2-HOPO) and its hexadentate analog TREN-(Me-3,2-HOPO), CP117, CP165, tachpyridine alkyl analogs [113], tachpyridine secondary amine linked analogs, tachpyridine pyridyl linked analogs, tachpyridine pyridyl linked maleimide derivative analogs [114], PIH, SIH, PCIH, PKIH, PIH analog compound 101, PIH analog compound 102, PIH analog compound 103, PIH analog compound 104, PIH analog compound 105, PIH analog compound 106, PIH analog compound 107, PIH analog compound 108, PIH analog compound 109, PIH analog compound 110, PIH analog compound 112, PIH analog compound 113, PIH analog compound 114, PIH analog compound 115, PIH analog compound 201, PIH analog compound 202, PIH analog compound 204, PIH analog compound 205, PIH analog compound 206, PIH analog compound 207, PIH analog compound 208, PIH analog compound 209, PIH analog compound 212, PIH analog compound 215, PIH analog compound 301, PIH analog compound 302, PIH analog compound 305, PIH analog compound 307, PIH analog compound 308, PIH analog compound 309, PIH analog compound 310, PIH analog compound 312, PIH analog compound 315, PCBH, PCHH, PCBBH, PCAH, PCTH, PKBH, PKAH, PK3BBH, PKHH, PKTH [115], 5-HP, Triapine, NT, N2 mT, N4 mT, N44 mT, N4eT, N4aT, N4pT, DpT, DP2 mT, Dp4 mT, Dp44 mT, Dp4eT, Dp4aT, Dp4pT, deferasirox (Exjade, ICL670A), a 5,5-diphenyl-1,2,4-triazole analog of deferasirox, HBED, Faralex-G, 4-hydroxy-2-nonylquinoline, and combinations thereof. Preferably, the iron chelator is selected from the group consisting of desferoxamine, deferasirox, and apotransferrin.

Another embodiment of the present invention is a kit for ameliorating an adverse effect in a patient caused by an acute transfusion into the patient of a composition comprising aged red blood cells. The kit comprises a container comprising an effective amount of an iron chelator packaged together with instructions on how to administer the iron chelator to the composition directly, to a blood product-related apparatus, or to a patient in need thereof.

As used herein, the term “blood product” refers to any composition that comprises red blood cells. Examples of such blood products include, but are not limited to, whole blood and “packed red blood cells” or PRBCs (which are also referred to in the art as “packed cells”). PRBCs are generally made from whole blood by removing platelets and plasma to leave a preparation that comprises mainly red blood cells. PRBCs may also be leuko-reduced, a process in which white blood cells are removed from the blood. Most of the blood products used for transfusion in the U.S. are leukoreduced PRBCs. Evidence suggests that some, but not all, of the adverse effects observed with the transfusion of older, stored blood are due to leukocytes in the blood product. The blood products used in accordance with the present invention are preferably leukoreduced.

In one aspect of this embodiment, the blood product-related apparatus is a blood filter or a blood bag.

In another aspect of this embodiment, the iron chelator is selected from the group consisting of apotransferrin, lactotransferrin, metalloenzymes, an hydroxamic acid polymer, a phosphorylated myo-inositol polymer, heme B, heme A, heme C, desferoxamine (DFO), desferrithiocin (DFT), desferri-exochelin (D-Exo), (S)-DMFT, (S)-DADMDFT, (S)-DADFT, 4′-(OH)-DADFT, 4′-(OH)-DADMDFT or its hexadentate derivative BDU, deferiprone (L1), an hydroxypyridinone ester prodrug whose metabolism yields a hydroxypyridinone analog, CP94, CP502, CP365, CP102, CP41, CP38, LiNAII, Pr-(Me-3,2-HOPO) and its hexadentate analog TREN-(Me-3,2-HOPO), CP117, CP165, tachpyridine alkyl analogs, tachpyridine secondary amine linked analogs, tachpyridine pyridyl linked analogs, tachpyridine pyridyl linked maleimide derivative analogs, PIH, SIH, PCIH, PKIH, PIH analog compound 101, PIH analog compound 102, PIH analog compound 103, PIH analog compound 104, PIH analog compound 105, PIH analog compound 106, PIH analog compound 107, PIH analog compound 108, PIH analog compound 109, PIH analog compound 110, PIH analog compound 112, PIH analog compound 113, PIH analog compound 114, PIH analog compound 115, PIH analog compound 201, PIH analog compound 202, PIH analog compound 204, PIH analog compound 205, PIH analog compound 206, PIH analog compound 207, PIH analog compound 208, PIH analog compound 209, PIH analog compound 212, PIH analog compound 215, PIH analog compound 301, PIH analog compound 302, PIH analog compound 305, PIH analog compound 307, PIH analog compound 308, PIH analog compound 309, PIH analog compound 310, PIH analog compound 312, PIH analog compound 315, PCBH, PCHH, PCBBH, PCAH, PCTH, PKBH, PKAH, PK3BBH, PKHH, PKTH, 5-HP, Triapine, NT, N2 mT, N4 mT, N44 mT, N4eT, N4aT, N4pT, DpT, DP2 mT, Dp4 mT, Dp44 mT, Dp4eT, Dp4aT, Dp4pT, deferasirox (Exjade, ICL670A), a 5,5-diphenyl-1,2,4-triazole analog of deferasirox, HBED, Faralex-G, 4-hydroxy-2-nonylquinoline, and combinations thereof. Preferably, the iron chelator is selected from the group consisting of desferoxamine, deferasirox, and apotransferrin.

A further embodiment of the present invention is a method for ameliorating an adverse effect in a patient caused by an acute transfusion into the patient of a composition comprising aged red blood cells. This method comprises providing an iron chelator, which is capable of chelating iron released by macrophage phagocytosis of the aged red blood cells, wherein the chelator ameliorates the adverse effect in the patient.

In one aspect of this embodiment, the adverse effect is a cytokine storm. As used herein, “cytokine storm” means an intense pro-inflammatory cytokine response, with elevated levels of various cytokines, such as MCP-1, IL-8, IL-6, TNF-α, IFN-γ, and IL-10.

In another aspect of this embodiment, the adverse effect is an increase in iron-dependent pathogens in the patient. In the present invention, an “iron-dependent pathogen” is any biological agent that causes a disease or an illness to its host, particularly a human, and that utilizes or otherwise processes iron. Non-limiting representative examples of iron-dependent pathogens according to the present invention include iron-dependent viruses, iron-dependent bacteria, iron-dependent fungi, and iron-dependent prions.

In a further aspect of this embodiment, the iron chelator is selected from the group consisting of peptides, polymers, small organic or inorganic molecules and combinations thereof.

As used herein, the terms “peptide,” “polypeptide,” and “protein” are used interchangeably. In the present invention, these terms mean a linked sequence of two or more amino acids, which may be natural, synthetic, or a modification or combination of natural and synthetic.

As used herein, “polymers” mean two or more molecules (other than amino acids) linked together to form higher order structures, including but not limited to long chains.

In the present invention, the term “small molecule” includes any chemical or other moiety, other than peptides and polymers, that can act as an iron chelator. Small molecules can include any number of therapeutic agents presently known and used, or that can be synthesized in a library of such molecules for the purpose of screening for iron chelating function. Small molecules are distinguished from macromolecules by size. The small molecules of the present invention usually have a molecular weight less than about 5,000 daltons (Da), preferably less than about 2,500 Da, more preferably less than 1,000 Da, most preferably less than about 500 Da.

Small molecules include without limitation organic molecules and inorganic molecules. As used herein, the term “small organic molecules” refer to any carbon-based small molecules other than macromolecules such as carbon-based polymers and polypeptides, and the term “inorganic molecules” refer to any other small molecules. In addition to carbon, small organic molecules may contain calcium, chlorine, fluorine, copper, hydrogen, iron, potassium, nitrogen, oxygen, sulfur and other elements. A small organic molecule may be in an aromatic or aliphatic form. Non-limiting examples of small organic molecules include acetones, alcohols, anilines, carbohydrates, monosaccharides, amino acids, nucleosides, nucleotides, lipids, retinoids, steroids, proteoglycans, ketones, aldehydes, saturated, unsaturated and polyunsaturated fats, oils and waxes, alkenes, esters, ethers, thiols, sulfides, cyclic compounds, heterocyclic compounds, imidazoles, and phenols. A small organic molecules as used herein also includes nitrated organic compounds and halogenated (e.g., chlorinated) organic compounds.

Preferred small molecules are relatively easier and less expensively manufactured, formulated or otherwise prepared. Preferred small molecules are stable under a variety of storage conditions. Preferred small molecules may be placed in tight association with macromolecules to form molecules that are biologically active and that have improved pharmaceutical properties. Improved pharmaceutical properties include changes in circulation time, distribution, metabolism, modification, excretion, secretion, elimination, and stability that are favorable to the desired biological activity. Improved pharmaceutical properties include changes in the toxicological and efficacy characteristics of the chemical entity.

In one preferred embodiment, the iron chelating peptide is selected from the group consisting of apotransferrin, lactotransferrin, metalloenzymes, iron-binding domains from such proteins, and synthetic peptides designed to mimic the iron-binding site of such proteins.

In another preferred embodiment, the iron chelating polymer is an hydroxamic acid polymer or a phosphorylated myo-inositol polymer.

In a further aspect of this embodiment, the iron chelator is a porphyrin ring selected from the group consisting of heme B, heme A, and heme C. As used herein, a “porphyrin ring” means a heterocyclic aromatic molecule characterized by the presence of four modified pyrrole subunits interconnected at their a carbon atoms via methine bridges (═CH—).

In yet another aspect of this embodiment, the iron chelator is a siderophore or a synthetically derived analog thereof. As used herein, a “siderophore” means an iron-binding compound secreted by microbes in response to the insoluble nature of iron in the environment. Preferably, the siderophore is selected from the group consisting of desferoxamine (DFO), desferrithiocin (DFT), and desferri-exochelin (D-Exo).

In an additional aspect of this embodiment, the iron chelator is a DFT analog selected from the group consisting of (S)-DMFT, (S)-DADMDFT, (S)-DADFT, 4′-(OH)-DADFT, and 4′-(OH)-DADMDFT or its hexadentate derivative BDU.

In a further aspect of this embodiment, the iron chelator is a hydroxypyridinone. As used herein, a “hydroxypyridinone” means a organic compound containing a heterocyclic 6-membered ring, a ketone group, and a hydroxyl group. Preferably, the hydroxypyridinone is selected from deferiprone (L1) or its analogs or an hydroxypyridinone ester prodrug whose metabolism yields a hydroxypyridinone analog. More preferably, wherein the deferiprone analog is selected from the group consisting of CP94, CP502, CP365, CP102, CP41, CP38, LiNAII, Pr-(Me-3,2-HOPO) and its hexadentate analog TREN-(Me-3,2-HOPO), and the hydroxypyridinone ester prodrug is selected from the group consisting of CP117 and CP165.

In yet another aspect of this embodiment, the iron chelator is a tachpyridine or an analog thereof. As used herein, a “tachpyridine” means a hexadentate iron chelator based on a cis,cis-1,3,5-triaminocyclohexane scaffold. Preferably, the tachpyridine analog is selected from the group consisting of tachpyridine alkyl analogs, tachpyridine secondary amine linked analogs, tachpyridine pyridyl linked analogs, and tachpyridine pyridyl linked maleimide derivative analogs.

In an additional aspect of this embodiment, the iron chelator is an aroylhydrazone. As used herein, a “aroylhydrazone” means a compound with the structure R₁R₂C═NNHR₃, wherein R₁, R₂, and/or R₃ contain an aromatic ring. Preferably, the aroylhydrazone iron chelator is selected from the group consisting of PIH, SIH, 311 series analog compounds, PCIH, PKIH, and analogs of each parent compound. Non-limiting examples of PIH analog include 100 series analog compounds 101, 102, 103, 104, 105, 106, 107, 108, 109, 110, 112, 113, 114 and 115; or 200 series analog compounds 201, 202, 204, 205, 206, 207, 208 209, 212, and 215 (See e.g., Kalinowski et al. [115]). Non-limiting examples of 311 series analog compounds include compounds 301, 302, 305, 307 308, 309, 310, 312, and 315 (See e.g., Kalinowski et al. [115]). Non-limiting examples of PCIH analogs include PCBH, PCHH, PCBBH, PCAH and PCTH. Non-limiting examples of PKIH analogs include PKBH, PKAH, PK3BBH, PKHH, and PKTH.

In a further aspect of this embodiment, the iron chelator is a thiosemicarbazone. As used herein, a “thiosemicarbazone” mean a compound having the following general structure:

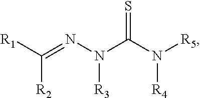

and which is capable of chelating iron. Preferably, the thiosemicarbazone is selected from the group consisting of 5-HP, Triapine, members of the NT series, and members of the DpT series. Non-limiting examples of the NT series include NT, N2 mT, N4 mT, N44 mT, N4eT, N4aT, and N4pT (See e.g., Kalinowski et al. [115]). Non-limiting examples of the DpT series include DpT, DP2 mT, Dp4 mT, Dp44 mT, Dp4eT, Dp4aT, and Dp4pT (See e.g., Kalinowski et al. [115]).

In another aspect of this embodiment, the iron chelator is selected from the group consisting of deferasirox (Exjade, ICL670A), a 5,5-diphenyl-1,2,4-triazole analog of deferasirox, HBED, Faralex-G, and 4-hydroxy-2-nonylquinoline (See e.g., Kalinowski et al. [115]).

In an additional aspect of this embodiment, the providing step comprises administering to the patient an amount of the iron chelator that is effective to ameliorate the adverse effect.

In yet another aspect of this embodiment, the providing step comprises, prior to transfusion, contacting the composition comprising aged red blood cells with an amount of the iron chelator that is effective to ameliorate the adverse effect.

In the present invention, an “effective amount” (or an amount “that is effective to”) is an amount sufficient to effect beneficial or desired results. In terms of treatment of a patient, preferably mammal, more preferably a human, an “effective amount” of an iron chelator is an amount sufficient to ameliorate the adverse effects in a patient caused by an acute transfusion. An effective amount can be administered in one or more doses.

A suitable, non-limiting example of a dosage of an iron chelator according to the present invention is from about 1 ng/kg to about 1000 mg/kg if it is administered to the patient, such as from about 1 mg/kg to about 100 mg/kg, including from about 5 mg/kg to about 50 mg/kg. Other representative dosages of an iron chelator include about 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg, 175 mg/kg, 200 mg/kg, 250 mg/kg, 300 mg/kg, 400 mg/kg, 500 mg/kg, 600 mg/kg, 700 mg/kg, 800 mg/kg, 900 mg/kg, or 1000 mg/kg. Preferably, the dosage is about 20 mg/kg body weight. In an alternative preferred, but exemplary embodiment, the iron chelator is administered at a dosage between about 30 to about 50 mg/kg/day, although lower doses, such as about 25 mg/kg/day are also possible. The effective dose of a compound may be administered as two, three, four, five, six or more sub-doses, administered separately at appropriate intervals throughout the day.

The effective amount is generally determined by a physician on a case-by-case basis and is within the skill of one in the art. Several factors are typically taken into account when determining an appropriate dosage. These factors include age, sex and weight of the patient, the condition being treated, the severity of the condition and the form of the drug being administered.

Effective dosage forms, modes of administration, and dosage amounts may be determined empirically, and making such determinations is within the skill of the art. It is understood by those skilled in the art that the dosage amount will vary with the route of administration, the rate of excretion, the duration of the treatment, the identity of any other drugs being administered, the age, size, and species of animal, and like factors well known in the arts of medicine and veterinary medicine. In general, a suitable dose of an iron chelator according to the invention will be that amount of the iron chelator, which is the lowest dose effective to produce the desired effect. The effective dose of an iron chelator maybe administered as two, three, four, five, six or more sub-doses, administered separately at appropriate intervals throughout the day.

An iron chelator of the present invention may be administered in any desired and effective manner: as pharmaceutical compositions for oral ingestion, or for parenteral or other administration in any appropriate manner such as intraperitoneal, subcutaneous, topical, intradermal, inhalation, intrapulmonary, rectal, vaginal, sublingual, intramuscular, intravenous, intraarterial, intrathecal, or intralymphatic. Further, an iron chelator of the present invention may be administered in conjunction with other treatments. An iron chelator of the present invention maybe encapsulated or otherwise protected against gastric or other secretions, if desired.

While it is possible for an iron chelator of the invention to be administered alone, it is preferable to administer the iron chelator as a pharmaceutical formulation (composition). Such pharmaceutical formulations typically comprise one or more modulators as an active ingredient in admixture with one or more pharmaceutically-acceptable carriers and, optionally, one or more other compounds, drugs, ingredients and/or materials. Regardless of the route of administration selected, the iron chelator of the present invention is formulated into pharmaceutically-acceptable dosage forms by conventional methods known to those of skill in the art. See, e.g., Remington's Pharmaceutical Sciences (Mack Publishing Co., Easton, Pa.).

[Pharmaceutically acceptable carriers are well known in the art (see, e.g., Remington's Pharmaceutical Sciences (Mack Publishing Co., Easton, Pa.) and The National Formulary (American Pharmaceutical Association, Washington, D.C.)) and include sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and tryglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, silicylate, etc. Each pharmaceutically acceptable carrier used in a pharmaceutical composition comprising an iron chelator of the invention must be “acceptable” in the sense of being compatible with the other ingredients of the formulation and not injurious to the subject. Carriers suitable for a selected dosage form and intended route of administration are well known in the art, and acceptable carriers for a chosen dosage form and method of administration can be determined using ordinary skill in the art.

Pharmaceutical compositions comprising an iron chelator of the invention may, optionally, contain additional ingredients and/or materials commonly used in pharmaceutical compositions. These ingredients and materials are well known in the art and include (1) fillers or extenders, such as starches, lactose, sucrose, glucose, mannitol, and silicic acid; (2) binders, such as carboxymethylcellulose, alginates, gelatin, polyvinyl pyrrolidone, hydroxypropylmethyl cellulose, sucrose and acacia; (3) humectants, such as glycerol; (4) disintegrating agents, such as agar-agar, calcium carbonate, potato or tapioca starch, alginic acid, certain silicates, sodium starch glycolate, cross-linked sodium carboxymethyl cellulose and sodium carbonate; (5) solution retarding agents, such as paraffin; (6) absorption accelerators, such as quaternary ammonium compounds; (7) wetting agents, such as cetyl alcohol and glycerol monosterate; (8) absorbents, such as kaolin and bentonite clay; (9) lubricants, such as talc, calcium stearate, magnesium stearate, solid polyethylene glycols, and sodium lauryl sulfate; (10) suspending agents, such as ethoxylated isostearyl alcohols, polyoxyethylene sorbitol and sorbitan esters, microcrystalline cellulose, aluminum metahydroxide, bentonite, agar-agar and tragacanth; (11) buffering agents; (12) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; (13) inert diluents, such as water or other solvents; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monosterate, gelatin, and waxes; (18) opacifying agents; (19) adjuvants; (20) wetting agents; (21) emulsifying and suspending agents; (22), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; (23) propellants, such as chlorofluorohydrocarbons and volatile unsubstituted hydrocarbons, such as butane and propane; (24) antioxidants; (25) agents which render the formulation isotonic with the blood of the intended recipient, such as sugars and sodium chloride; (26) thickening agents; (27) coating materials, such as lecithin; and (28) sweetening, flavoring, coloring, perfuming and preservative agents. Each such ingredient or material must be “acceptable” in the sense of being compatible with the other ingredients of the formulation and not injurious to the subject. Ingredients and materials suitable for a selected dosage form and intended route of administration are well known in the art, and acceptable ingredients and materials for a chosen dosage form and method of administration may be determined using ordinary skill in the art.

Pharmaceutical compositions suitable for oral administration may be in the form of capsules, cachets, pills, tablets, powders, granules, a solution or a suspension in an aqueous or non-aqueous liquid, an oil-in-water or water-in-oil liquid emulsion, an elixir or syrup, a pastille, a bolus, an electuary or a paste. These formulations may be prepared by methods known in the art, e.g., by means of conventional pan-coating, mixing, granulation or lyophilization processes.

Solid dosage forms for oral administration (capsules, tablets, pills, dragees, powders, granules and the like) may be prepared by mixing the active ingredient(s) with one or more pharmaceutically-acceptable carriers and, optionally, one or more fillers, extenders, binders, humectants, disintegrating agents, solution retarding agents, absorption accelerators, wetting agents, absorbents, lubricants, and/or coloring agents. Solid compositions of a similar type maybe employed as fillers in soft and hard-filled gelatin capsules using a suitable excipient. A tablet may be made by compression or molding, optionally with one or more accessory ingredients. Compressed tablets may be prepared using a suitable binder, lubricant, inert diluent, preservative, disintegrant, surface-active or dispersing agent. Molded tablets may be made by molding in a suitable machine. For example, an iron chelator according to the present invention may be in the form of a tablet having about 125 mg, 250 mg, or 500 mg of active ingredient. The tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein. They may be sterilized by, for example, filtration through a bacteria-retaining filter. These compositions may also optionally contain opacifying agents and may be of a composition such that they release the active ingredient only, or preferentially, in a certain portion of the gastrointestinal tract, optionally, in a delayed manner. The active ingredient can also be in microencapsulated form.

Liquid dosage forms for oral administration include pharmaceutically-acceptable emulsions, microemulsions, solutions, suspensions, syrups and elixirs. The liquid dosage forms may contain suitable inert diluents commonly used in the art. Besides inert diluents, the oral compositions may also include adjuvants, such as wetting agents, emulsifying and suspending agents, sweetening, flavoring, coloring, perfuming and preservative agents. Suspensions may contain suspending agents.

Pharmaceutical compositions for rectal or vaginal administration may be presented as a suppository, which maybe prepared by mixing one or more active ingredient(s) with one or more suitable nonirritating carriers which are solid at room temperature, but liquid at body temperature and, therefore, will melt in the rectum or vaginal cavity and release the active compound. Pharmaceutical compositions which are suitable for vaginal administration also include pessaries, tampons, creams, gels, pastes, foams or spray formulations containing such pharmaceutically-acceptable carriers as are known in the art to be appropriate.

Dosage forms for the topical or transdermal administration include powders, sprays, ointments, pastes, creams, lotions, gels, solutions, patches, drops and inhalants. The active compound may be mixed under sterile conditions with a suitable pharmaceutically-acceptable carrier. The ointments, pastes, creams and gels may contain excipients. Powders and sprays may contain excipients and propellants.

Pharmaceutical compositions suitable for parenteral administrations comprise one or more iron chelators in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain suitable antioxidants, buffers, solutes which render the formulation isotonic with the blood of the intended recipient, or suspending or thickening agents. Proper fluidity can be maintained, for example, by the use of coating materials, by the maintenance of the required particle size in the case of dispersions, and by the use of surfactants. These compositions may also contain suitable adjuvants, such as wetting agents, emulsifying agents and dispersing agents. It may also be desirable to include isotonic agents. In addition, prolonged absorption of the injectable pharmaceutical form may be brought about by the inclusion of agents which delay absorption. In one preferred embodiment, the iron chelator may be infused subcutaneously over about 8 to about 12 hours.

In some cases, in order to prolong the effect of a drug containing an iron chelator of the present invention, it is desirable to slow its absorption from subcutaneous or intramuscular injection. This may be accomplished by the use of a liquid suspension of crystalline or amorphous material having poor water solubility.

The rate of absorption of the drug then depends upon its rate of dissolution which, in turn, may depend upon crystal size and crystalline form. Alternatively, delayed absorption of a parenterally-administered drug may be accomplished by dissolving or suspending the drug in an oil vehicle. Injectable depot forms may be made by forming microencapsule matrices of the active ingredient in biodegradable polymers. Depending on the ratio of the active ingredient to polymer, and the nature of the particular polymer employed, the rate of active ingredient release can be controlled. Depot injectable formulations are also prepared by entrapping the drug in liposomes or microemulsions which are compatible with body tissue. The injectable materials can be sterilized for example, by filtration through a bacterial-retaining filter.

The formulations may be presented in unit-dose or multi-dose sealed containers, for example, ampules and vials, and may be stored in a lyophilized condition requiring only the addition of the sterile liquid carrier, for example water for injection, immediately prior to use. Extemporaneous injection solutions and suspensions may be prepared from sterile powders, granules and tablets of the type described above.

The following examples are provided to further illustrate the compositions and methods of the present invention. These examples are illustrative only and are not intended to limit the scope of the invention in any way.

### EXAMPLES

### Example 1

### Storage of Leukoreduced Mouse and Human RBCs is Similar

Mouse blood was obtained aseptically by cardiac puncture into a standard storage solution used for humans: CPDA-1 (Baxter, Deerfield, Ill.). Whole blood from 30-50 mice was leukoreduced using a pediatric leukoreduction filter (Purecell Neo, Pall Corp., Port Washington, N.Y.), centrifuged, and stored at a 60-75% hematocrit at 4° C. for up to 21 days. Twenty-one or fewer days was selected for mouse RBCs (rather than ≦35 days used with humans) because the normal mouse RBC lifespan is approximately half that of human RBCs [86]. Pre-storage leukoreduction of mouse RBCs achieved at least a 3-log₁₀ reduction in leukocytes (LeucoCOUNT kit, BD Biosciences, San Jose, Calif.; not shown).

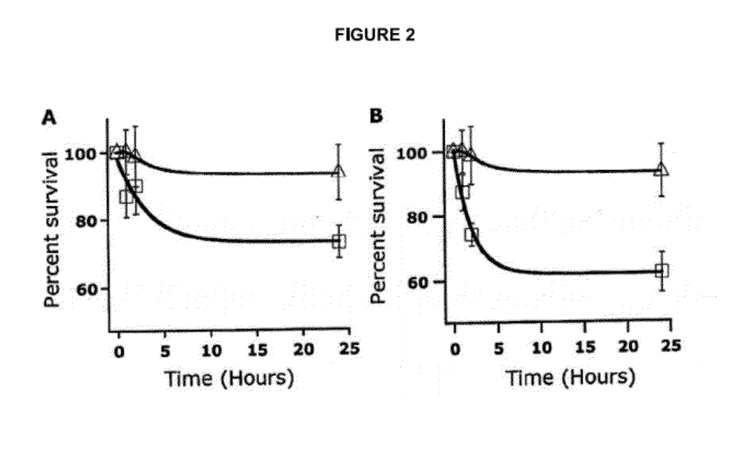

RBC survival studies in mice were performed using ⁵¹Cr-labeling [87]. The 24-hour post-transfusion survival of 2 and 3 week old stored mouse RBCs was 73% (±5%) and 62% (±6%), respectively, whereas that of fresh RBCs was 93% (±8%) (mean (±1 SD) (FIG. 2). To ensure sterility during storage, all materials were disposable and pyrogen free. In addition, before each experiment, 500 μL of stored RBCs were inoculated into Peds Plus/F culture bottles (BD Diagnostic Systems, Franklin Lakes, N.J.) and bacterial growth detected with a BACTEC™ blood culture system (BD Diagnostic Systems) for >5 days; all samples were negative. As noted previously, the FDA mandates that, on average, 75% of stored RBCs survive for 24 hours post-transfusion. Thus, mouse RBCs stored for ≦2 weeks in CPDA-1 using the methods disclosed here would be acceptable using the criteria of this FDA standard.

### Transfusion of Leukoreduced, Stored RBCs Induces a Pro-Inflammatory Response.

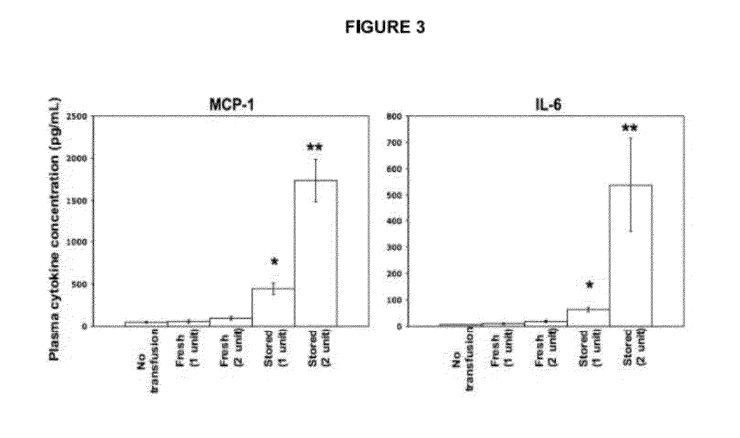

Statistically significant and dose-responsive increases in plasma levels of MCP-1, IL-6 (FIG. 3), KC (the mouse homolog of IL-8), MIP-1β, and TNF-α (not shown) were each observed 2 hours after transfusion of older stored RBCs (13 mice/group; p<0.05 (Student's 2-tailed t-test), when comparing results from transfusion of stored RBCs to fresh RBCs). In contrast, no significant differences were observed for IL-10 or IFN-γ (not shown).

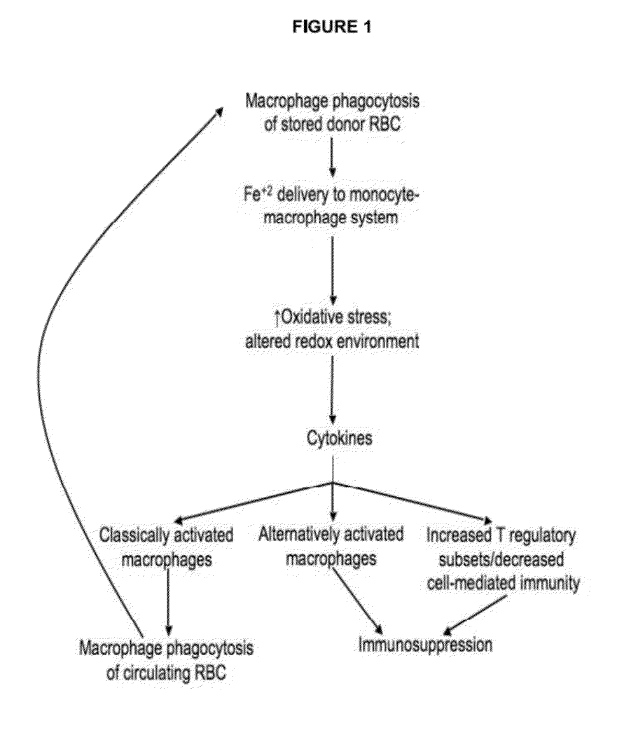

Interestingly, when mice were transfused with older stored RBCs, they had dramatic increases in pro-inflammatory cytokines (especially MCP-1) by 2 hours post-transfusion (FIG. 3). This suggests that one possible consequence of the “early” pro-inflammatory response induced by transfusion of older stored RBCs is a “late,” systemic, anti-inflammatory response that predisposes patients to subsequent infection (FIG. 1). Indeed, surviving a pro-inflammatory insult, such as an infection, requires a controlled immune response that limits collateral damage to self [52].

### Stored RBCs are Cleared via Phagocytosis by Kupffer Cells.

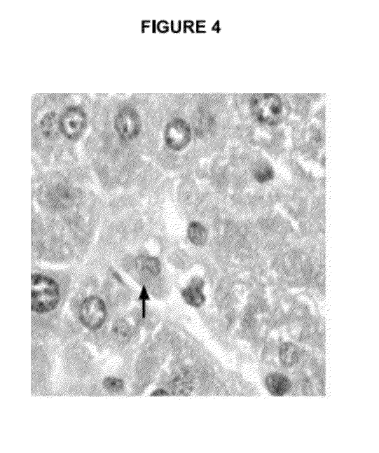

Mice were transfused with RBCs stored for 3 weeks, sacrificed, and liver sections were examined by light microscopy. Kupffer cells with ingested RBCs were frequently seen (>1/high power field) in mice transfused with older stored RBCs (FIG. 4), but rarely seen in mice transfused with fresh RBCs (not shown). Thus, older stored RBCs are cleared by phagocytosis in vivo.

### Transfusion of Stored RBCs Increases Iron Content in Liver, Spleen, and Kidney.

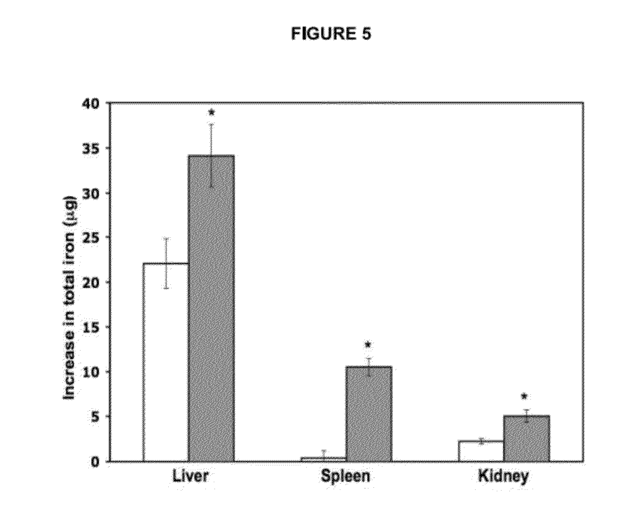

Total iron was measured at necropsy in various organs by a wet ashing procedure [88] following transfusion of fresh or stored RBCs (dose of 400 μL, 13 mice per group). Total iron was significantly increased in the liver, spleen, and kidney of mice transfused with stored RBCs (p<0.05, 2-tailed Student t-test) (FIG. 5). Thus, rapid clearance of substantial amounts of stored RBCs leads to iron deposition in liver, spleen, and kidney. In a typical experiment, about 140 μg of total iron is transfused into each mouse. Based on the RBCs survival data (FIG. 2), at 2 hours post-transfusion, about 25% of 2-weeks stored RBCs are cleared. Thus, about 35 μg of iron is delivered to the monocyte-macrophage system; this is approximately equivalent to the amount of excess iron recovered from the spleen, kidney, and liver of these mice (FIG. 5).

### The Transfusion-Induced Pro-Inflammatory Cytokine Response Requires Intact, Hemoglobin-Containing RBCs.

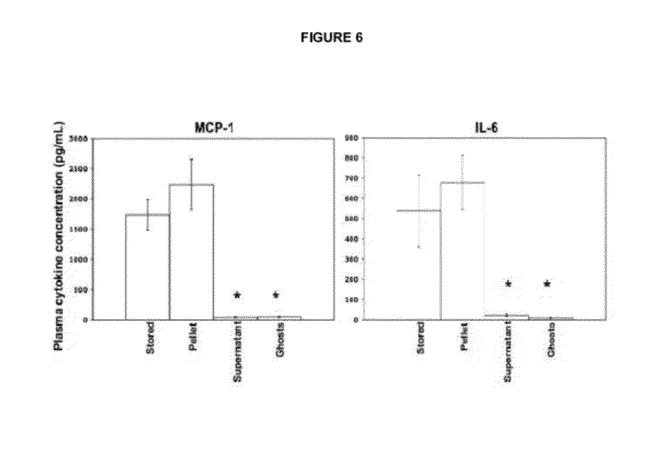

Studies were performed to determine whether acute delivery of membrane-encapsulated hemoglobin iron is required to induce a cytokine storm, rather than additives in the storage medium or factors in the supernatant of stored RBCs. To this end, a comparison was made of the effects of transfusing saline-washed 2-week stored RBCs, the supernatant from stored RBCs, and RBC ghosts derived from stored RBCs. The stored RBCs were washed 3 times in 10 volumes of normal saline and then re-suspended in saline to a final hemoglobin concentration equivalent to that of the unwashed stored RBCs. RBC ghosts, prepared by hypotonic lysis of stored RBCs [89], were extensively washed until a white pellet was obtained. The concentration of ghosts was quantified by flow cytometry using Trucount (BD Biosciences) to verify that equivalent numbers of ghosts, stored RBCs, and washed stored RBCs were transfused. In addition, the ghosts contained only about 0.07% of the total iron present in an equivalent number of stored RBCs (not shown). Mice (6-13 per group) were transfused with normalized amounts of fresh RBCs, stored RBCs, supernatant from stored RBCs, the saline-washed stored RBC pellet, or ghosts derived from stored RBCs. A pro-inflammatory cytokine response was only found after transfusion of either stored RBCs or the saline-washed stored RBC pellet; MCP-1 and IL-6 results are shown as examples (FIG. 6). Qualitatively similar and statistically significant results were obtained for KC, MIP-1β, and TNF-α (not shown); no significant differences between the groups were seen for IL-10 and IFN-γ (not shown). Because RBC ghosts did not induce a pro-inflammatory response, this suggests that delivery of sufficient amounts of hemoglobin iron is required to produce this phenomenon. In addition, although transfusing intact RBCs in the saline-washed pellet induced a similar cytokine response, the supernatant did not. This suggests that the pro-inflammatory response is not derived from a compound that accumulates in the supernatant during storage, such as commercial additives, RBC-derived vesicles, cytokines, or non-transferrin-bound iron.

### Transfusion of Stored RBCs Acutely Increases Plasma Non-Transferrin-Bound Iron Levels.

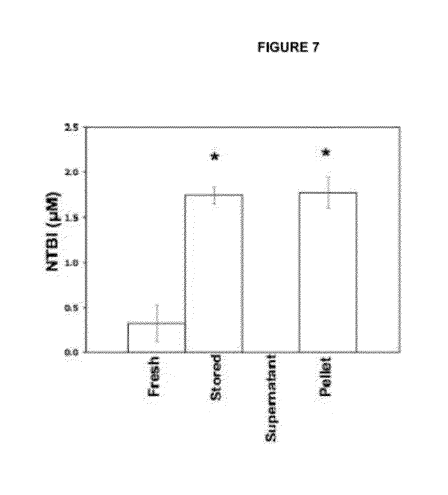

Plasma non-transferrin-bound iron was quantified following transfusion of fresh RBCs, 2-week stored RBCs, the washed RBC pellet, or supernatant from stored RBCs (400 μL, 5-7 mice/group). Plasma non-transferrin-bound iron was only elevated in mice transfused with either stored RBCs or the washed RBC pellet (FIG. 7). Because non-transferrin-bound iron may induce harmful effects due to its redox potential, this suggests that the intact transfused RBCs are the source of these increased levels, presumably by increased egress of iron after phagocytosis of older stored RBCs.

Although currently the importance of concomitant infusion of non-transferrin-bound iron cannot be excluded when transfusing humans with older units of stored RBCs, transfusing mice with older stored RBCs did lead to post-transfusion increases in plasma levels of non-transferrin-bound iron (FIG. 7), as noted above. Interestingly, washing older stored mouse RBCs did not prevent either induction of a post-transfusion pro-inflammatory cytokine response (FIG. 6) or increases in circulating non-transferrin-bound iron levels (FIG. 7). In addition, transfusion of the supernatant derived from stored mouse RBCs did not either induce a cytokine response (FIG. 6) or increase non-transferrin-bound iron levels at 2 hours post-transfusion (FIG. 7). Taken together, this suggests that the increased post-transfusion non-transferrin-bound iron levels in mice resulted from increased egress of iron from macrophages following phagocytosis of older stored RBCs.

### Transfusion of Stored RBCs Exacerbates and Prolongs the Cytokine Storm Induced by LPS.

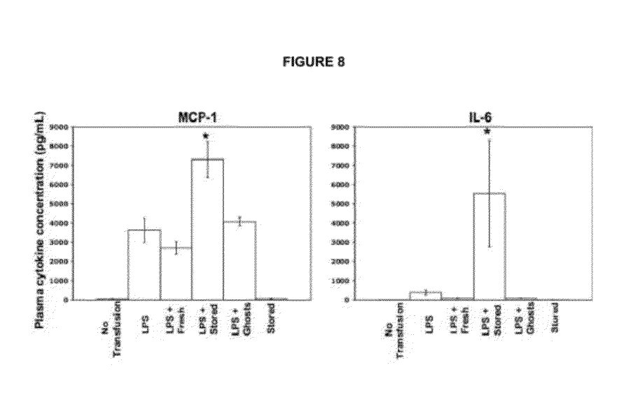

C57BL/6 mice (5-10/group) were injected with a sub-lethal dose of LPS, with or without concurrent transfusion of either fresh RBCs, RBCs stored for 2 weeks, or ghosts prepared from stored RBCs. Mice were sacrificed 24 hours post-transfusion and cytokines measured (FIG. 8). By 24 hours post-transfusion, LPS-treated mice that were transfused with stored RBCs maintained markedly elevated levels of many pro-inflammatory cytokines, including KC, MIP-1β, and TNF-α; as examples, the MCP-1 and IL-6 results are shown in FIG. 8. In addition, all LPS-treated mice transfused with stored RBCs were moribund by 24 hours post-transfusion, lacking spontaneous movement and exhibiting a slow righting reflex; all other groups of mice appeared well at this time point. Finally, transfusion of hemoglobin-free ghosts did not enhance the LPS-induced cytokine storm in mice. Taken together, this suggests that rapid clearance of stored RBCs synergizes with LPS to exacerbate and prolong the cytokine storm; therefore, this mouse model replicates human studies that implicate transfusions of older stored RBCs in adverse effects in patients, such as those with sepsis.

### Example 2

### Mice

Wildtype C57BL/6 and FVB/NJ mice were purchased from the Jackson Laboratory (Bar Harbor, Me.). SAA1-luciferase reporter mice were obtained from Caliper Life Sciences (Hopkinton, Mass.). Mice were used at 8-12 weeks of age. Procedures were approved by the appropriate Institutional Animal Care and Use Committees.

### Mouse RBC Collection, Storage, and Derivatives.

FVB/NJ and C57BL/6 mice were bled aseptically by cardiac puncture into citrate phosphate dextrose-adenine-1 (CPDA-1) obtained directly from di-(2-ethylhexyl)phthalate-plasticized polyvinyl chloride human primary collection packs (product code 4R3611; Baxter). The final CPDA-1 concentration used for storage was 14%. Whole blood collected from 30-50 mice was pooled and leukoreduced using a Neonatal High Efficiency Leukocyte Reduction Filter (Purecell Neo, Pall Corp.), centrifuged (400 g for 15 minutes), and volume reduced to a final hemoglobin level of 17.0-17.5 g/dL (as determined by a modified Drabkin's assay [106] at a 1:251 dilution of stored RBCs to Drabkin's reagent (Ricca Chemical Company, Arlington, Tex.), optical density measured at 540 nm and compared to Count-a-part Cyanmethemoglobin Standards Set (Diagnostic Technology, Inc., Belrose, Australia). Residual leukocytes were enumerated by flow cytometry (LeucoCOUNT kit, BD Biosciences). The stored RBCs were placed in 15 mL Falcon tubes, sealed with parafilm, and stored in the dark at 4° C. for up to 14 days. On the day of transfusion, 500 μl of stored RBCs were inoculated into Peds Plus/F culture bottles (BD Diagnostic Systems) and bacterial growth detected with a BACTEC™ continuous monitoring blood culture system (BD Diagnostic Systems) for up to 5 days or until bacterial growth was detected (this method detects at least 10 colony forming units (CFU) per milliliter with a sensitivity of 97%).

Washed stored RBCs were prepared with 3 washes using 10 volumes of phosphate-buffered saline (PBS) and centrifugation at 400×g. After the final wash, the washed stored RBCs were resuspended in PBS to a final hemoglobin concentration of 17.0 to 17.5 g/dL for transfusion. Supernatant was obtained using a 400×g spin of stored RBCs and 400 μL of this solution were transfused undiluted. RBC ghosts were obtained by hypotonic lysis of twice the volume of stored RBCs (i.e., for 400 μL of ghosts, 800 μL of stored RBCs were hemolyzed) with PBS to distilled water (1:15), followed by multiple washes with the same buffer and centrifugation at 30,000×g until a white pellet was obtained. The white pellet of RBC ghosts was resuspended in PBS. Stroma-free RBC lysate was prepared by freeze-thaw of washed stored RBCs followed by centrifugation at 16,000×g to pellet and remove the stroma.

### Transfusion and Short-Term RBC Survival.

RBCs (200 or 400 μL at 17.0-17.5 g/dL of hemoglobin; 1 or 2 equivalent human units, respectively) were transfused through the retro-orbital plexus of isoflurane-anesthetized mice. The proportion of transfused RBCs circulating at 2 and 24 hours posttransfusion (i.e., the 2- and 24-hour posttransfusion survival) was measured by either a dual- or a single-labeling method (preliminary studies confirmed that there is no significant difference in these methods for the conditions of this study (not shown)). For dual labeling, an aliquot of fresh, syngeneic C57BL/6 RBCs was labeled with chloromethylbenzamido 1,1′-dioctadecyl-3,3,3′,3′-tetramethylindocarbo-cyanine perchlorate (Dil; Invitrogen, Carlsbad, Calif.) and an aliquot of allogeneic, FVB/NJ fresh RBCs or stored RBCs was labeled with 3,3′-dihexadecyloxacarbocyanine perchlorate (DiO; Invitrogen), as described previously [102]. At defined time points post-transfusion, 1-2 μL of blood was obtained from the tail vein and transferred to 500 μL of PBS for flow cytometric detection of fluorescently-labeled RBCs. Survival was calculated by comparing the ratio of Dil- to DiO-labeled RBCs in the sample to the ratio in the transfuse itself. For single label studies, a 10% aliquot of fresh RBCs or stored RBCs was labeled with DiO. To determine percent survival, the ratio of DiO-labeled RBCs to unlabeled RBCs acquired with a FACSCalibur® flow cytometer (BD Biosciences), was compared between a 10-minute post-transfusion sample and a sample obtained at the final endpoint. At a defined time point (2 hours or 24 hours post-transfusion), all mice were anesthetized with isoflurane, sacrificed, and blood was obtained by cardiac puncture using heparinized syringes. Washed stored RBCs were prepared by washing 3 times using 10 volumes of PBS and centrifugation at 400 g. Following the final wash, washed stored RBCs were re-suspended in PBS to a final hemoglobin concentration of 17.0-17.5 g/dL. Supernatant was obtained following a 400×g spin of stored RBCs and 400 μL of this solution was transfused undiluted. RBCs ghosts were obtained by hypotonic lysis of twice the volume of stored RBCs (i.e. for 400 μL of ghosts, 800 μL of stored RBCs were hemolysed) with PBS:dH₂O (1:15), followed by multiple washes with the same buffer and centrifugation at 30,000×g until a white pellet was obtained. The white pellet of RBC ghosts was re-suspended in PBS. For some experiments, LPS (E. coli 0111:B4 (Sigma); 30-100 μg per mouse) dissolved in 100 μL PBS was injected by tail vein into mice immediately prior to transfusion. LPS treated mice were video recorded prior to sacrifice using a Canon PowerShot SD600. In some experiments, 3 mg of deferoxamine (DFO, Novartis) dissolved in 100 μL PBS, or 3 mg of DFO preincubated for 1 hour with an equimolar concentration of ferric citrate (Sigma-Aldrich), were injected into the tail vein of mice immediately before transfusion. Finally, in some experiments, 2 mg of liposomal clodronate or PBS-liposomes (both from Encapsula NanoSciences LLC, Nashville, Tenn.) were injected intraperitoneally into mice 48 hours before transfusion.

### Histology and Immunohistochemistry

At necropsy, the liver and spleen were removed, fixed overnight with 10% neutral-buffered formalin, and embedded in paraffin. Sections were stained with hematoxylin and eosin or were deparaffinized and immunostained with an anti-mouse F4/80 monoclonal antibody (eBioscience, San Diego, Calif.) at a 1:500 dilution, followed by biotinylated anti-rat secondary antibody (1:200 dilution), ABC reagent (1:50 dilution), and development with a 3,3′-diaminobenzidine substrate kit (all from Vector Laboratories, Burlingame, Calif.). Images were captured using an Olympus BX40 microscope and a SPOT INSIGHT digital camera (Diagnostic Instruments, Sterling Heights, Mich.).

### Inflammatory Protein Measurements.

Cytokines/chemokines, including interleukin-6 (IL-6), interleukin-10 (IL-10), monocyte chemoattractant protein-1 (MCP-1), interferon-γ (IFN-γ), tumor necrosis factor-α (TNF-α), macrophage inhibitory protein-1β (MIP-1β), and keratinocyte-derived chemokine/CXCL1 (KC/CXCL1) were quantified using the Cytometric Bead Array Mouse Flex Kit (BD Biosciences). Heparinized plasma, obtained by cardiac puncture, was analyzed at 1:4 and/or 1:10 dilutions. Flow cytometry data, acquired with a FACSCalibur® flow cytometer (BD Biosciences), were analyzed using FlowJo software (Tree Star, Inc., Ashland, Oreg.). Plasma serum amyloid A (SAA) levels were measured using a mouse SAA ELISA Kit (Life Diagnostics, Inc., West Chester, Pa.) following the manufacturer's instructions.

### Iron-Related Measurements.

Plasma NTBI was measured by a nitrilotriacetic acid (NTA) ultrafiltration assay [107]. In brief, heparinized plasma (90 μL) was incubated with 800 mM NTA, pH 7.0, at room temperature for 30 minutes. Plasma proteins were removed by ultrafiltration (NanoSep, 30-kDa cutoff, polysulfone type (Pall Life Sciences)); 10,620×g at 15° C. for 45 minutes) and iron in the ultrafiltrate was determined by a ferrozine assay [95]. Total organ iron was measured using a wet ashing procedure [88]. In brief, the wet weight of organs obtained at necropsy was quantified; the entire spleen or portions of liver (about 100 mg) or kidney (about 80 mg) were placed in 2 mL glass vials. Following desiccation at 65° C. for 24 hours, 200 μl of acid mixture (70% perchloric acid:nitric acid 2:1) were added. After drying for 5-6 hours at 182° C., 1 mL of 3M HCl was added and mixed. The acidified sample (50 μL) was then incubated for 30 minutes with 200 μL of chromogen (1.6 mM bathophenanthroline, 2 M sodium acetate, and 11.5 mM thioglycolic acid). Absorbance of samples and iron standards at 535 nm was measured in duplicate and mean values used for calculating total organ iron. Hemoglobinemia was detected spectrophotometrically using a PowerWave XS spectrophotometer (BioTek, Winooski, Vt.).

### In Vivo Imaging of Luciferase Activity.

Male SAA1-luciferase transgenic mice [108] were transfused by tail-vein injection with 200 μL of fresh RBCs (<24 hours storage) or stored RBCs. Bioluminescence imaging was performed using an In Vivo Imaging System (Caliper Life Sciences), as described [108]. Mice were anesthetized with isoflurane, injected i.p. with 150 mg/kg luciferin (Caliper Life Sciences), and imaged 10 minutes later for 1-60 seconds. Photons emitted from specific regions were quantified using LivingImage software (Caliper Life Sciences); luciferase activity is expressed as photons per second.

### In Vitro Bacterial Growth.

A pathogenic strain of E. coli, obtained from an anonymous patient with a urinary tract infection, was used. For each experiment, a sample from a frozen stock of this E. coli was inoculated into Nutrient Broth (Difco, BD Biosciences) and grown to mid-log phase (about 3 hours). Bacteria were then washed twice in PBS and re-suspended to about 200,000 colony forming units (CFU)/μL. Five microliters of bacterial suspension were then added to 100 μL of heparinized plasma in a 96-well EIA/RIA plate (Costar, Sigma). Bacterial growth was measured by absorbance at 600 nm. In some experiments, 20 μM of ferric citrate or sodium citrate (Sigma), bovine serum albumin, 2,2′-dipyridyl (all from Sigma-Aldrich), protoporphyrin IX (Frontier Scientific, Logan, Utah), or DFO were added to plasma before bacterial inoculation.

### Statistical Analysis.

Significance between two means was calculated using a two-tailed Mann-Whitney U test. Significance relevant to bacterial growth in vitro was determined by converting each growth curve to an area under the curve (AUC) value followed by a 2-tailed Mann-Whitney U test to compare mean AUC for each group. A value for P less than 0.05 was considered significant. Statistical analysis was performed using Prism 5 (GraphPad Software, Inc., La Jolla, Calif.).

### Results.

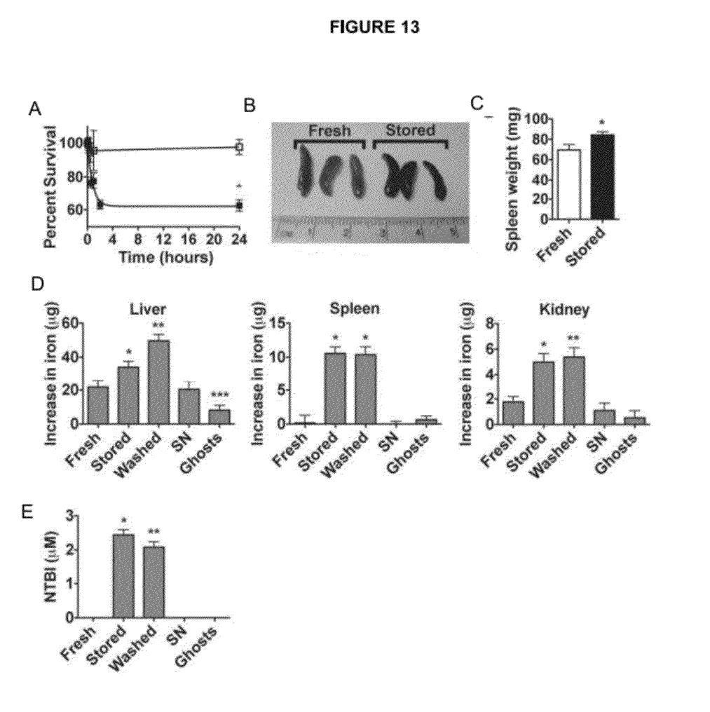

Post-transfusion survival of leukoreduced C57BL/6 mouse RBCs stored for up to 2 weeks in a standard preservative solution, CPDA-1, is comparable to the FDA standards at outdate [109]. In the current study, donor FVB/NJ mouse RBCs were used to model an allogeneic transfusion. RBCs were leukoreduced before storage (>3-log₁₀ leukocyte reduction (not shown)) and aliquots had no microbial growth after incubation in blood culture media for 5 days. The 24-hour survival of fresh (i.e., <24-hour of storage) and 2-week stored allogeneic FVB/NJ RBCs transfused into C57BL/6 mice was similar to the syngeneic transfusion results (FIG. 13A) [109]. For the experiments presented hereafter, the mean 2-hour survival of fresh RBCs was 100.1% (s.e.m. 3.8) as compared to 83.6% (s.e.m. 4.7) for stored RBCs; all stored RBCs were transfused after 2 weeks of storage.

To determine the fate of the hemoglobin iron cleared after transfusion of stored RBCs, tissue iron levels were measured at necropsy 2-hours following transfusion of (i) fresh RBCs, (ii) stored RBCs, (iii) washed stored RBCs, (iv) supernatant prepared from stored RBCs, and (v) ghosts derived from stored RBCs. Washed stored RBCs were re-suspended in PBS so that the amount of hemoglobin transfused was similar to that in fresh RBCs and stored RBCs (200 or 400 μL containing 17.0-17.5 g/dL of hemoglobin per transfusion). Supernatant and stored RBC-derived ghosts contained an average hemoglobin of 1.19 g/dL (s.e.m. 0.48) and <0.02 g/dL, respectively. As compared with fresh RBC transfusions, mean total iron was significantly increased in liver (12.1 μg), spleen (10.1 μg), and kidney (2.8 μg) following stored RBC transfusions (FIG. 13d). In a typical experiment, about 225 μg of total iron were transfused per mouse (calculated as the amount of iron in 400 μl of RBCs containing 17.5 g/dL of hemoglobin). Based on survival data, 16.4% of stored RBCs were cleared by 2-hours post-transfusion, resulting in about 36 μg of iron cleared from the circulation; thus, the excess iron recovered in spleen, kidney, and liver of these mice together accounts for about 70% of the total iron delivered. Bone marrow iron was not measured. In addition, stored clearance produced splenic discoloration at necropsy (FIG. 13B) and increased spleen weight (FIG. 13C). No significant differences in liver or kidney weight were detected (not shown). Finally, only stored RBCs and washed stored RBCs transfusions increased plasma NTBI levels 2-hour post-transfusion (FIG. 13E). This surge in plasma NTBI was short-lived, as plasma NTBI levels were undetectable by 24-hour after transfusion of stored RBCs.

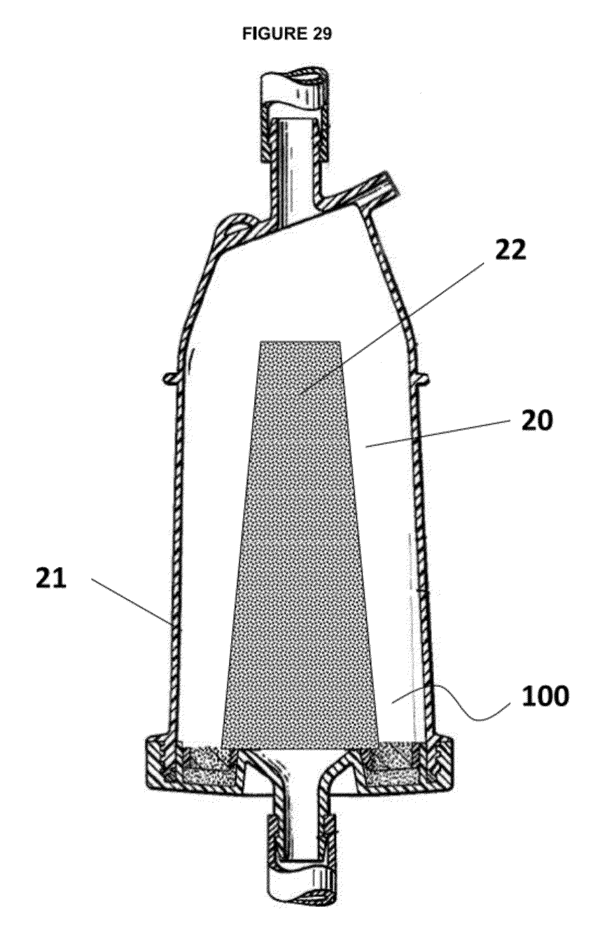

To determine whether macrophages were responsible for clearing stored RBCs in this model, mice were treated with liposomal clodronate or control PBS-liposomes 48 hours before transfusion. The 2-hour RBC survival was significantly increased in liposomal clodronate-treated mice compared with the PBS-liposomal control (FIG. 30A). Liposomal clodronate treatment depleted hepatic and splenic (FIG. 30B) macrophages, as assessed by immunohistochemistry for the F4/80 mouse macrophage marker. In nonclodronate-treated control animals transfused with syngeneic stored RBCs, histologic examination showed increased erythrophagocytosis by hepatic (FIG. 30C) and splenic (data not shown) macrophages, which was confirmed by F4/80 staining of macrophages (FIG. 30C).

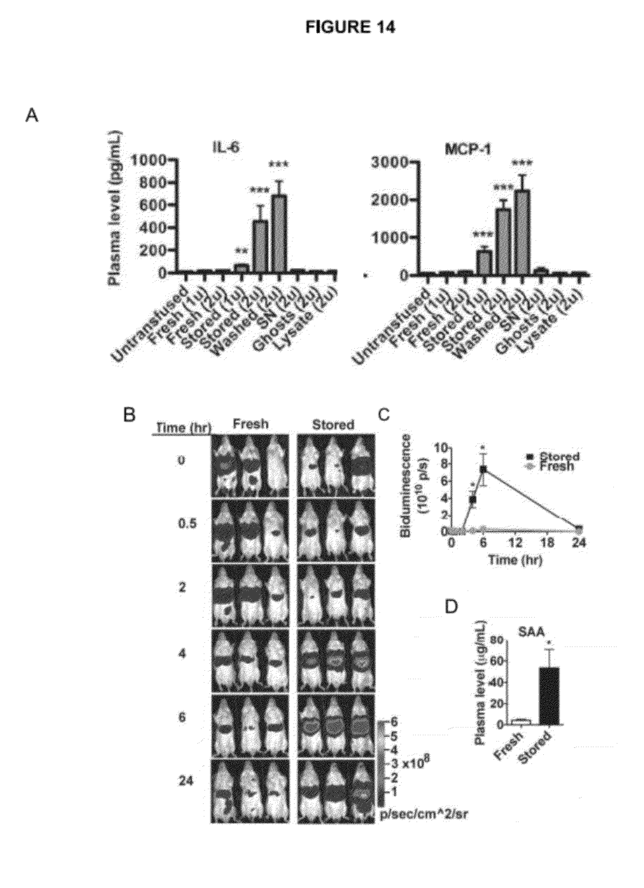

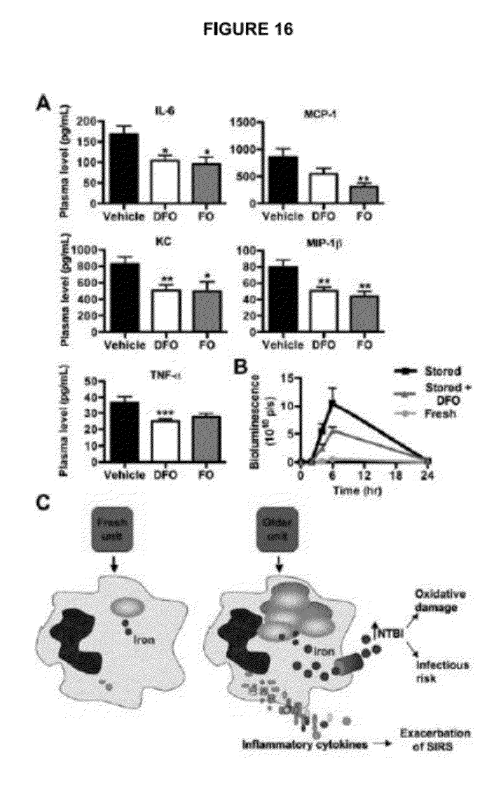

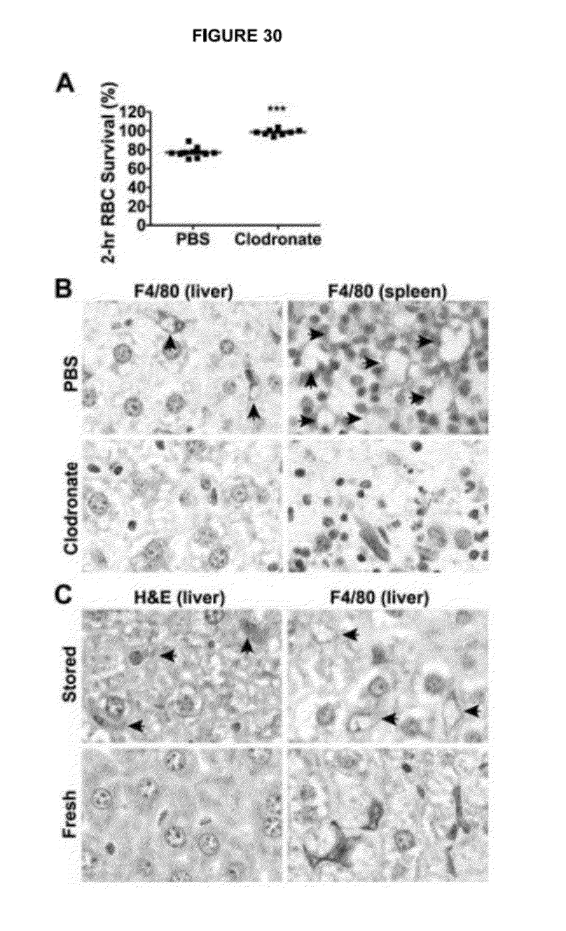

To determine whether rapid stored RBC clearance induces inflammation, and whether membrane-encapsulated hemoglobin iron is required, rather than factors accumulating in the stored RBC supernatant, recipient mice were transfused with normalized amounts of (i) fresh RBCs, (ii) stored RBCs, (iii) washed stored RBCs, (iv) stored RBC-derived supernatant, (v) ghosts prepared from stored RBCs, or (vi) stroma-free stored RBC lysate. At 2 hours after transfusion, mice transfused with stroma-free stored RBC lysate had dramatic hemoglobinemia (FIG. 31) and hemoglobinuria (data not shown), compared with mice transfused with intact RBCs. By 2-hours post-transfusion, a dose-responsive pro-inflammatory cytokine response was detected only after transfusion of either stored RBCs or washed stored RBCs. As examples, circulating interleukin (IL)-6 and monocyte chemoattractant protein (MCP)-1 levels are shown in FIG. 14A. Statistically significant increases were seen with CXCL1 (i.e. KC), macrophage inflammatory protein (MIP)-1β, and tumor necrosis factor (TNF)-α (FIG. 17); no significant differences were seen with IL-10 or interferon (IFN)-γ (FIG. 17). Qualitatively similar cytokine results were obtained after transfusion of stored syngeneic RBCs (i.e., from C57BL/6 donors; data not shown).

The lack of response to transfusions of stored RBC-derived ghosts suggests that hemoglobin is required to produce inflammation. In addition, intact washed stored RBCs, but not supernatant, induced this cytokine response; therefore, compounds accumulating in the supernatant during storage (e.g. cytokines, RBC-derived vesicles, cell-free hemoglobin, NTBI) were not responsible. Finally, the elicited cytokine pattern differs from that induced by lipopolysaccharide (LPS) (e.g. increased IL-10 and IFN-γ with LPS; not shown), and infusing stored RBC supernatant did not induce cytokines; therefore, the results of transfusing stored RBCs and washed stored RBCs are not due to inadvertent LPS contamination, but due to the transfused stored RBCs themselves.

To further investigate the inflammatory response after stored RBC transfusion, male transgenic serum amyloid A1 (SAA1)-luciferase reporter mice [108] were transfused with 200 μL of fresh RBCs or stored RBCs. SAA1 is an acute phase reactant induced by elevated levels of pro-inflammatory cytokines [108]. Only stored RBC transfusions induced a robust luciferase signal in the hepatosplenic region (>300-fold over baseline, FIGS. 14B, C) as measured by noninvasive bioluminescent imaging. Expression was detectable at 4-hour post-transfusion and returned to baseline by 24-hours post-transfusion. Plasma SAA1 protein levels 24-hours post-transfusion were consistent with the imaging results (FIG. 14D).

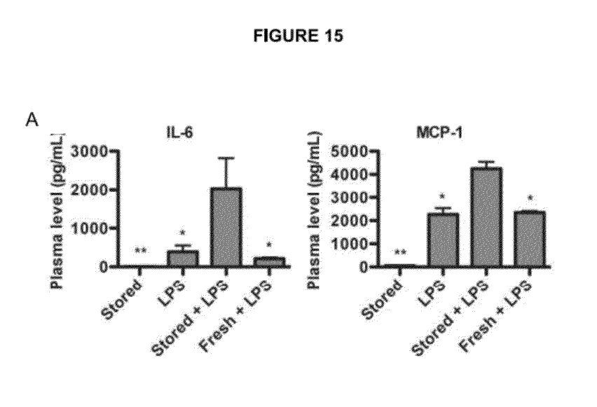

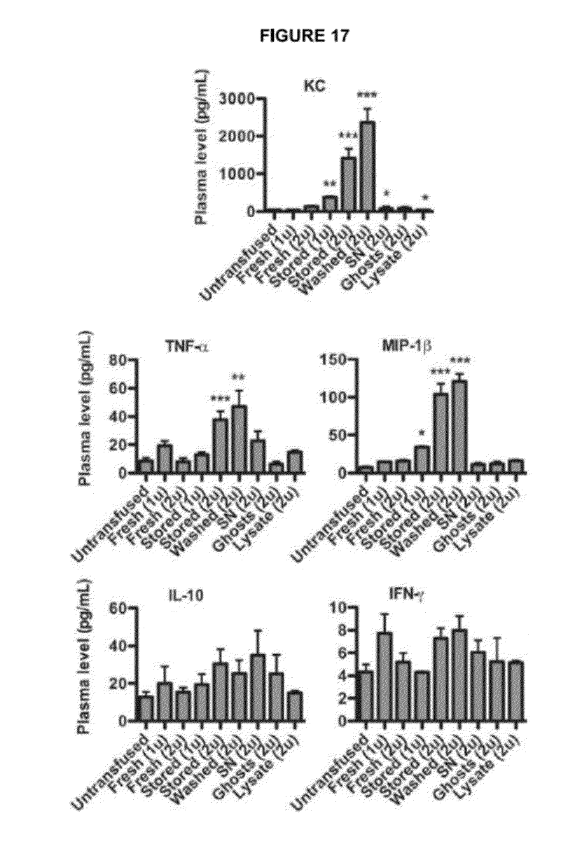

Although stored RBC transfusions induced a significant pro-inflammatory response, no mice developed clinically apparent symptoms, such as anorexia, reduced mobility, decreased alertness, or lack of grooming. Nonetheless, it is believed that the inflammatory response to stored RBC transfusion could exacerbate a pre-existing inflammatory state and result in clinical symptoms, potentially explaining the relationship between critically ill patients, older stored RBC transfusions, and adverse outcomes [11, 12]. Thus, recipient mice were injected with a sub-clinical dose of LPS, with or without concurrent fresh RBC or stored RBC transfusions. Following sacrifice at 24-hours post-transfusion (or earlier if moribund), cytokines were measured. LPS-treated mice transfused with stored RBCs maintained markedly elevated levels of multiple pro-inflammatory cytokines, including IL-6, MCP-1 (FIG. 15A), KC, MIP-1β, IFN-γ, and IL-10 (FIG. 18). In addition, in experiments with higher LPS doses, LPS-treated mice transfused with stored RBCs were moribund by 18-24 hours post-transfusion, lacking spontaneous movement and exhibiting a slow righting reflex, whereas all other groups of mice appeared much less ill and exhibited spontaneous movement and grooming (not shown). Taken together, these results suggest that rapid stored RBC clearance synergizes with LPS to exacerbate and prolong the cytokine storm.

To determine whether transfusion of stored RBCs enhances pathogen growth in vitro, heparinized plasma samples obtained from mice post-transfusion were inoculated in vitro with a pathogenic strain of E. coli and growth was measured by turbidity. Plasma obtained from mice 2-hours post-transfusion with either stored RBCs or washed stored RBCs showed significantly increased bacterial growth as compared to that from untransfused mice or mice transfused with fresh RBCs, supernatant derived from stored RBCs, or ghosts prepared from stored RBCs (FIG. 15B). This was an acute effect, because plasma collected 24-hours after stored RBC transfusion did not enhance bacterial growth (FIG. 15B). Total iron in pooled plasma 2-hours post-transfusion with fresh RBCs or stored RBCs was 176 μg/dL or 295 μg/dL, respectively (i.e. increased by about 20 μM after stored RBC transfusion). When 20 μM of iron citrate, but not sodium citrate (20 μM), bovine serum albumin (80 μM), or protoporphyrin IX (20 μM), was added to pooled plasma from mice transfused with fresh RBCs, bacterial growth was promoted to a similar level as in plasma from mice transfused with stored RBCs (FIG. 15C). This suggests that increased circulating iron induced by stored RBC transfusion is responsible for the increased bacterial growth.

Conversely, when 20 μM of an iron chelator, DFO, was added to pooled plasma from mice transfused with stored RBCs, bacterial growth was partially inhibited (FIG. 15D). This inhibition was due to the iron-binding capacity of DFO, because preincubation of DFO with an equimolar amount of ferric citrate (i.e., producing ferroxamine [FO]) prevented the inhibition of bacterial growth. A more dramatic inhibition of bacterial growth was probably not achieved because some types of bacteria can use FO as an iron source [117]. Nonetheless, a greater inhibition of bacterial growth was achieved using higher concentrations of the bidentate ferrous iron chelator, 2,2′-dipyridyl [118] (FIG. 15E). This inhibitory effect was similarly abrogated when the 2,2′-dipyridyl was preincubated with a one-third molar ratio of ferric citrate.

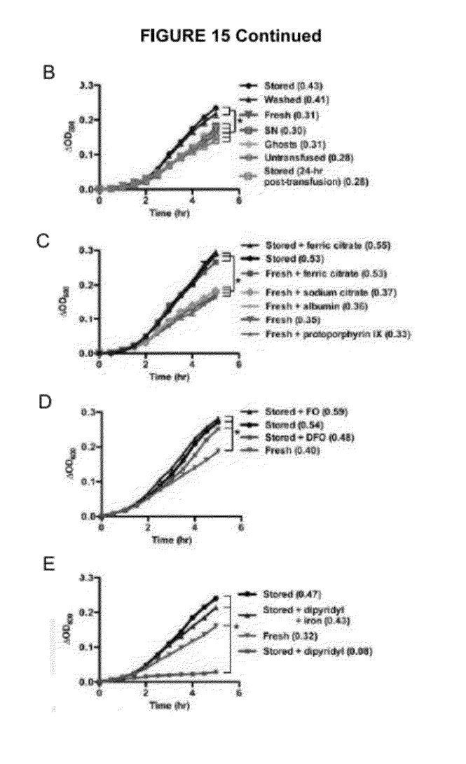

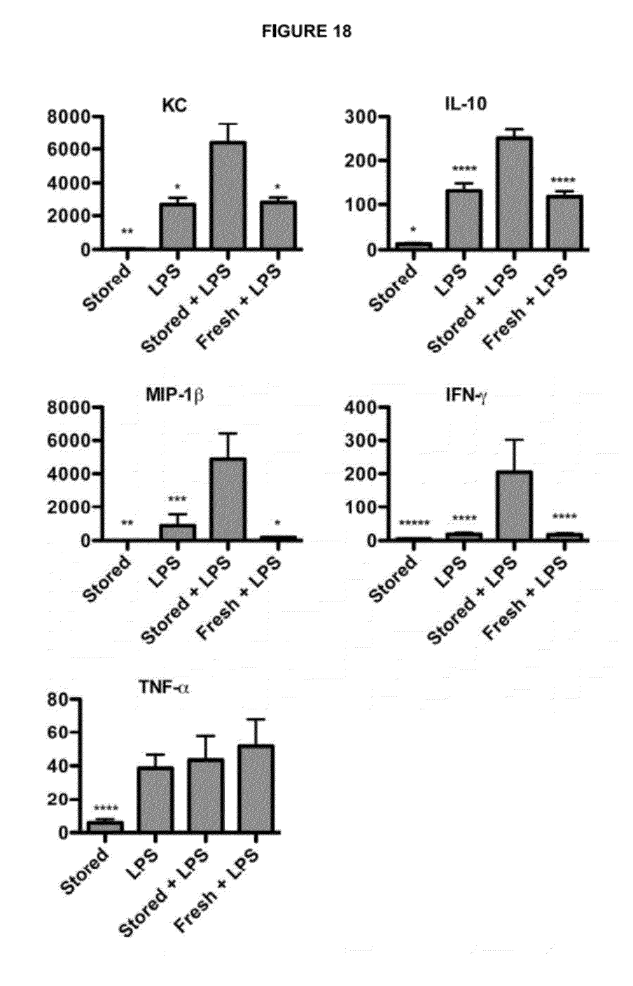

Finally, to examine whether administration of an iron chelator can ameliorate the pro-inflammatory response to stored RBC transfusions, mice were infused intravenously with 3 mg (about 120 mg/kg) of deferoxamine (DFO), an FDA-approved iron chelator, immediately before transfusion. Iron chelation prevented increases in plasma IL-6 levels (from 212.8±30.8 pg/mL to 98.4±10.1 pg/mL; mean±s.e.m.; P=0.005; FIG. 16A) and showed a trend towards reducing MCP-1 (FIG. 16A), KC, and TNF-α levels (FIG. 19). Thus, DFO significantly inhibited increases in proinflammatory cytokine levels (FIG. 16A) and showed a trend toward reducing the luciferase signal in SAA1-luciferase reporter mice (FIG. 16B). However, SAA protein levels were not significantly different at 24 hours after transfusion (data not shown). The “iron hypothesis” model (FIG. 16C) disclosed herein may be used to explain the mechanisms underlying the adverse effects of stored RBC transfusions.

The effect of DFO could be attributed to either its iron chelating capacity or to other antioxidative properties it may possess, such as its ability to scavenge the hydroxyl radical [119], or to both. Thus, control experiments were performed by infusing FO (i.e., an equimolar combination of DFO and ferric citrate) followed by transfusion of stored RBCs (FIG. 16a). In this setting, FO was as effective as DFO at ameliorating the cytokine response.

The major conclusions derived from the current studies with mice are that transfusions of RBCs after prolonged storage induce a proinflammatory response, are associated with increased circulating NTBI levels, and lead to increased iron deposition in various tissues. The lack of a proinflammatory response to transfusions of either membrane ghosts or stroma-free lysate derived from stored RBCs suggests that membrane-encapsulated hemoglobin is required to produce inflammation.

The dramatic hemoglobinemia observed with transfusion of stroma-free RBC lysate (FIG. 31) did not result in an inflammatory cytokine response, suggesting that intravascular hemolysis is not responsible for this effect; rather extravascular hemolysis by macrophage-mediated phagocytosis is implicated.

In addition, intact washed stored RBCs, but not the associated supernatant, induced this cytokine response; therefore, transfusion of compounds accumulating in the supernatant during storage (e.g., cytokines, RBC-derived vesicles, cell-free hemoglobin, bioactive lipids, NTBI, etc.) was not responsible. The transfusion of stored RBCs also synergizes with LPS to exacerbate and prolong the cytokine storm. Finally, measuring bacterial growth in vitro suggests that the increased circulating iron released by clearance of transfused stored RBCs (i.e., NTBI) increases bacterial proliferation. Thus, we propose the iron underlying these adverse effects of stored RBC transfusions.

Although all RBC units used for transfusion were cultured after storage and no bacterial growth was detected, they were not tested for LPS contamination. The possibility of low-level bacterial contamination also exists. However, infusing supernatant or stroma-free lysate derived from stored RBC did not induce a cytokine response. In addition, injection of LPS alone induces a different cytokine profile (FIGS. 15A and 17). Therefore, the results obtained by transfusing stored RBCs or washed stored RBCs were probably not because of inadvertent LPS or bacterial contamination during RBC collection and processing; rather, the results were due to the transfused stored RBCs themselves.

Studies from the 1960s [120-121] suggest that erythrophagocytosis in mice by either antibody-mediated RBC clearance, phenylhydrazine treatment, or clearance of xenogeneic RBCs are each associated with an increased susceptibility to sepsis induced by various bacterial species, including E. coli. The mechanism for this effect was not elucidated at the time, but may now be potentially explained by the ferrophilia of these organisms [122] and the dramatic rise in circulating NTBI levels after RBC clearance as seen in our model of RBC storage and transfusion.

In recent studies with another murine transfusion model, prolonged storage of RBCs before transfusion into endotoxinemic mice caused increases in lung chemokines, neutrophils, and microvascular permeability [123]. Similar to our findings, this response was related to the RBCs themselves, as washing of the stored RBCs pre-transfusion did not abrogate the response. It is possible that the exacerbation of the existing lung inflammation seen in this model [123] may also involve increased NTBI levels after transfusion of stored RBCs. For example, when excess plasma iron is not sequestered by transferrin, the NTBI can participate in redox reactions leading to oxidative damage, cytotoxicity, and enhanced expression of endothelial adhesion molecules [124, 125]. Thus, NTBI may act as another pathologic factor in this lung injury model.

The finding that both DFO, a nonmembrane permeable chelator, and its iron-chelated form, FO, inhibit the cytokine response induced by transfusion of stored RBCs to a similar extent (FIG. 16) may be because of the antioxidant properties of DFO [119]. Indeed, a similar effect was seen when LPS-challenged mice were treated with DFO or FO; both reduced TNF-α levels to a similar extent [119]. Reactive oxygen species can mediate cytokine production by activating transcription factors, such as nuclear factor-B [126,127]; therefore, it is possible that reactive oxygen species produced after clearance of stored RBCs are responsible for the proinflammatory response and that DFO and FO ameliorate this pro-oxidant effect. The role of free intracellular iron, released by processing of the ingested RBCs, in producing these putative reactive oxygen species remains to be determined. The lack of a significant effect of DFO on SAA1 levels may be a result of variation in genetic background. The SAA1-luciferase transgenic mice are on the BALB/c background, whereas all other recipients in this study are on the C57BL/6 background. Additional studies are required to assess the effect of mouse strain on the inflammatory response to transfusions of older, stored RBCs.

The inventors previously observed that immunoglobulin G (IgG) antibody mediated RBC clearance induces a cytokine storm in a mouse model of incompatible RBC transfusion [102]. The same cytokine pattern was seen after transfusion of either stored RBCs or incompatible RBCs; however, the cytokine response in the former case is not as profound. Therefore, it is possible that Fcγ receptor-mediated signaling, which is involved in clearance of IgG-coated RBCs, amplifies the cytokine response in the incompatible transfusion model [128-130].

In sum, the current murine RBC storage and transfusion model provides evidence that transfusion of older stored RBCs produces a proinflammatory response that is associated with increased levels of tissue iron in the liver, spleen, and kidney, and increased circulating levels of NTBI. This indicates that the pro-oxidant effects of iron released after acute clearance of stored RBCs may be responsible for some of the harmful effects of RBC transfusion after prolonged storage. In addition, the presence of increased plasma NTBI levels provides a possible explanation for the increased risk of bacterial infection suggested by retrospective studies in humans after transfusion of stored RBCs [7, 12, 14, 131-132]. Preventing the pro-oxidant effects of iron derived by rapid clearance of transfused stored RBCs may decrease these adverse effects. With more than 15 million RBC transfusions annually in the United States alone, there are serious clinical implications of this iron hypothesis as it relates to human transfusion therapy.

### Example 3

### Iron Chelation Inhibits the Pro-Inflammatory Cytokine Response Induced in Mice by Transfusion of Older Stored RBCs

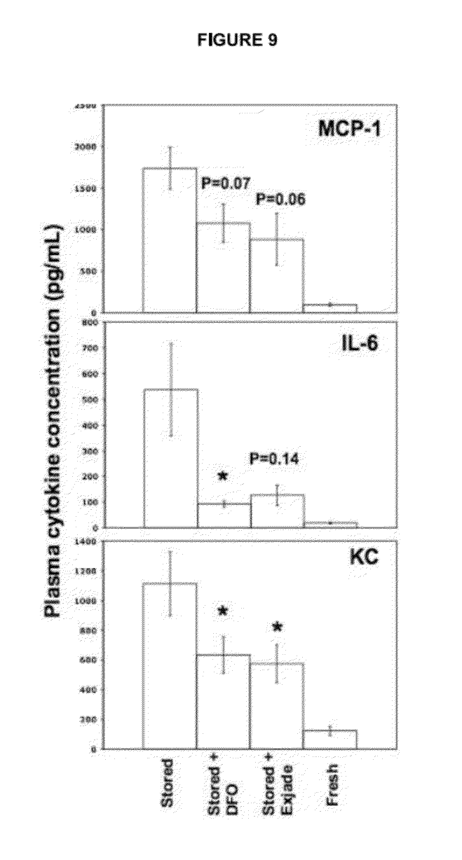

Proof-of-principle pre-clinical studies were performed to show that this approach will lead to innovative treatments to prevent adverse outcomes in recipients of older stored RBC transfusions. Thus, mice received 120 mg/kg of deferoxamine (DFO; Novartis, East Hanover, N.J.), an FDA-approved intravenous iron chelator, or 30 mg/kg of deferasirox (Exjade; Novartis), an FDA-approved, cell permeable, oral iron chelator, at 24 and 6 hours before RBC transfusion. Chelation statistically significantly blocked increases in plasma KC and IL-6 levels, and demonstrated a trend towards reducing MCP-1 levels (FIG. 9). Thus, new therapeutic interventions will result from confirming the animal data regarding the mechanism by which older stored RBC transfusions produce adverse effects.

### Example 4

Transfusion of Mouse RBCs Stored in AS-1 Induces Inflammation and Exacerbates Bacterial Sepsis

Although human observational studies suggest that transfusion of older, stored RBCs is associated with increased rates of bacterial sepsis, the relevant mechanisms are unknown. Using a mouse model of RBC storage and transfusion, the transfusion of mouse RBCs stored in CPDA-1 in plastic tubes enhances alloimmunization, induces a cytokine storm, and increases plasma levels of non-transferrin bound iron. Using a new mouse RBC storage system in AS-1 (Adsol preservative) and Di(2-ethylhexyl)phthalate (DEHP) plasticized storage bags, whether transfusion of stored, packed RBCs would exacerbate monobacterial sepsis was examined with a model ferrophilic pathogen.

RBCs from C57BL/6 donor mice were collected in citrate phosphate dextrose solution (CPD), pooled, filter leukoreduced, hard spun, plasma reduced, brought to a 60% hematocrit with AS-1, and stored in DEHP-plasticized storage bags (Fenwal, Inc., Lake Zurich, Ill.). RBCs stored for defined times and freshly-collected RBCs were labeled with lipohilic dyes (DiO and Dil), transfused into C57BL/6 recipients, and 24-hour post-transfusion RBC recovery (PTR) was determined.

Hemoglobin was quantified by Drabkin's assay. Cytokines in transfusion recipients were measured by a multiplex flow cytometric assay. Cohorts of mice infected intraperitoneally with 1000 colony forming units of Salmonella typhimurium, strain LT2 (ATCC), were transfused with 350 μl of fresh RBCs, 2-week stored RBCs, or no RBCs, and mouse survival was determined.

After 9 days of storage, the mean 24-hour PTR was 87.8% (SD 9.4%; n=10) with one mouse exhibiting a 24-hour PTR below the Food and Drug Administration (FDA) criterion of 75%. Hemolysis in vitro was 3.4% (i.e. hemoglobin in supernatant/total hemoglobin). Plasma levels of multiple pro-inflammatory cytokines (i.e. KC, MCP-1, and IL-6) were statistically significantly increased in mice at 4 hours after transfusion of 9-day stored RBCs. RBCs stored for 14 days had a mean 24-hour PTR of 32% (SD 4.2%; n=5) and transfusion recipients exhibited higher pro-inflammatory cytokine levels than those transfused 9 day old RBCs. Bacterially-infected mice (n=5 per group) transfused with 14-day stored RBCs survived for a median of 4 days, whereas mice transfused with fresh RBCs and non-transfused mice survived for a median of >14 days (p<0.01 Log-rank (Mantel-Cox) Test). At death, mice transfused with stored RBCs were severely bacteremic (>1×10⁴ bacteria/mL blood).

Thus, storage of mouse RBCs in AS-1 for 9 days in plastic bags more closely approximates physical human RBC storage conditions than previously published models. Transfusion of 14-day stored mouse RBCs, but not fresh RBCs, significantly exacerbated bacterial sepsis with a model pathogen.

### Example 5

### Transfusion of Older Stored RBCs into Healthy Individuals

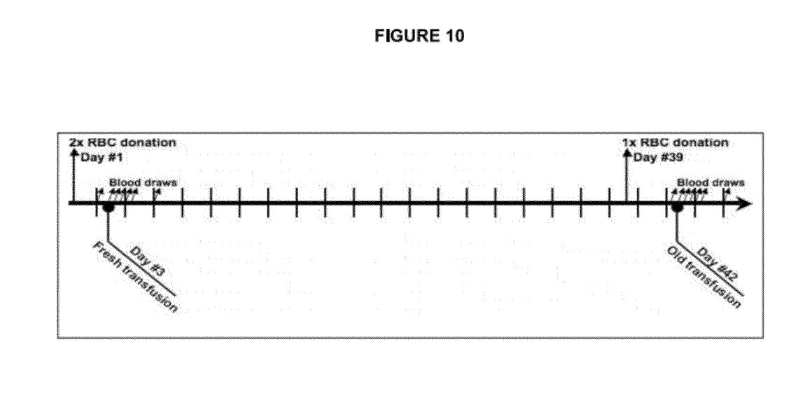

IRB approval was obtained from both Columbia University Medical Center (CUMC) and The New York Blood Center (NYBC) to perform a prospective study with 11 healthy human volunteers (see below for sample size justification). A schematic outline of the study for each volunteer is shown in FIG. 10. In brief, on Day #1 of the study for each participant, the volunteer will undergo an autologous double RBC unit donation by apheresis at the NYBC. The RBC donation will be pre-storage leukoreduced, split equally into two RBC storage bags, and stored in AS-1 in the CUMC Blood Bank. One unit will be transfused into the same participant “fresh” (i.e. on Day #3); the other unit will be transfused after the maximal allowable storage time (i.e. “old” on Day #42). Thus, each participant will receive autologous transfusions of fresh and older stored RBCs. Blood samples (about 20 mL each) will be drawn at various time points, as follows: prior to transfusion, immediately post-transfusion, and 1, 2, 4, 24, and 72 hour post-transfusion. Table 1 summarizes the types of analytes that will be measured at each time point; these are focused on markers of inflammation (e.g. cytokines), hemolysis (e.g. haptoglobin), iron metabolism (e.g. hepcidin), and relevant physiological systems (e.g. evaluating renal function using creatinine and blood urea nitrogen). Cytokines and iron-related analytes will also be measured in the RBC units pre-transfusion to determine whether levels detected in recipients are due to endogenous production in vivo.

TABLE 1
Circulating analytes measured at each time point

| Category | Analytes measured |
|---|---|
| Cytokines | MCP-1, IL-6, IL-8, IL-10, TNF-α, IFN-γ |
| Iron-related | Non-transferrin-bound iron, hepcidin, serum iron, total iron- |
| | binding capacity (i.e. serum transferrin saturation), ferritin, |
| | free hemoglobin and heme |
| Hemolysis | Lactate dehydrogenase, haptoglobin, direct and indirect |
| | bilirubin |
| Other | Complete blood count with absolute reticulocyte count and |
| | differential count, chemistry panel (e.g. blood urea |
| | nitrogen, creatinine, etc.) |

To prevent interference by transfusion-induced polycythemia on the effects of the older stored RBC transfusions, and to control for the possible effects of blood donation on subsequent cytokine responses to RBC transfusion, volunteers will be phlebotomized to collect 500 mL of whole blood on day #39 (i.e. 3 days prior to the final transfusion). This unit will be discarded (i.e. this unit will not be transfused into the recipient on Day #42). This will ensure as much control as possible in the “fresh” and “old” RBC transfusion settings.

### Study Sites.

Autologous RBC donations will be performed at one of five conveniently located, NYBC donation sites equipped with double RBC collection apheresis instruments (ALYX; Baxter). The autologous RBC units will be processed by the NYBC according to current Good Manufacturing Practice (cGMP) quality standards and transported to the CUMC Blood Bank for storage prior to storage Day #3.

The CUMC Blood Bank issues about 30,000 packed RBC units per year and will issue each autologous unit after a full cross-match and following CUMC Standard Operating Procedures. Transfusions will take place in the CUMC Outpatient Apheresis and Transfusion Suite, which is overseen by 4 experienced Transfusion Medicine attending physicians, staffed by 5 expert apheresis nurses, and supervised by an apheresis nurse with >30 years of experience. All required phlebotomy and transfusion equipment are available in this about 1,000 sq. ft. suite equipped with 8 beds. All transfusions will follow established CUMC Standard Operating Procedures.

All blood samples will be transported to the Center for Advanced Laboratory Medicine (CALM). CALM coordinates laboratory testing for clinical research studies at CUMC. As such, the CALM technical staff will provide coded labels for blood tubes, will spin tubes and aliquot samples as necessary, and will transport samples to their testing sites. For example, samples for standard clinical laboratory tests, such as complete blood counts, will be transported to the CUMC Clinical Laboratories for testing; samples for investigational testing, such as cytokine levels, will be aliquoted and stored at −80° C. until testing. CALM contains flexible laboratory space, computers for the management of results and stored specimens, refrigerators, -20° C. and -80° C. freezers, and liquid nitrogen storage facilities. In addition, all iron- and heme-related assays will be performed in the Iron Reference Laboratory in CALM. Cytokine levels will be measured. Finally, any residual samples will be aliquoted, frozen, and banked at −80° C. for future use.

### Selection of Subjects.

Healthy males, 18-65 years of age, who respond to flyers distributed throughout CUMC, will be recruited. The initial study will be restricted to 18-65 year old men to avoid confounding factors of age or gender (e.g. the female menstrual cycle can affect iron levels and cytokine responses). Participation in this study will be unrestricted with respect to ethnicity. All participants must fulfill the current, standard, NYBC requirements for volunteer double RBC donation (e.g. weight >130 lbs; height >5′1″; hemoglobin >13.3 g/dL; no significant past medical history that would preclude donation such as cardiac disease, major organ disease, or cancer; etc.). In addition, routine infectious disease testing will be performed at the NYBC for each donation. Any positive result will lead to exclusion due to the possibility that concurrent infectious disease can alter cytokine responses. Specific criteria for inclusion and exclusion are:

Inclusion criteria: (i) male, 18-65 years of age; (ii) body weight >130 lbs; (iii) height >5′1″; (iv) hemoglobin >13.3 g/dL.

Exclusion criteria: (i) ineligible for donation based on the NYBC blood donor questionnaire; (ii) systolic blood pressure >180 or <90 mm Hg, diastolic blood pressure >100 or <50 mm Hg; (iii) heart rate <50 or >100; (iv) temperature >99.5° F. prior to donation; (v) temperature >100.4° F. or subjective feeling of illness prior to transfusion (this is to avoid having a concurrent illness affect cytokine measurements post-transfusion); (vi) positive results on standard blood donor infectious disease testing.

### Cytokine Measurements.

The following will be measured in serum at each time point with a multiplex flow cytometric assay (CBA Flex kit; BD Biosciences): MCP-1, IL-8, IL-6, TNF-α, IFN-γ, and IL-10. These markers were selected based on the pre-clinical mouse studies (see Examples disclosed above). In addition, to identify compounds for future study, selected samples will be screened by the Human Inflammation Multi-Analyte Profile (Rules Based Medicine, Inc.) for 46 inflammatory mediators (including those listed above). The complete list of the 46 mediators are listed in Table 2 below

TABLE 2

| | |
|---|---|
| 1 | Alpha-1 Antitrypsin |
| 2 | Alpha-2 Macroglobulin |
| 3 | Beta-2 Microglobulin |
| 4 | Brain-Derived Neurotrophic Factor |
| 5 | C Reactive Protein |
| 6 | Complement 3 |
| 7 | Eotaxin |
| 8 | Factor VII |
| 9 | Ferritin |
| 10 | Fibrinogen |
| 11 | GM-CSF |
| 12 | Haptoglobin |
| 13 | Intercellular Adhesion Molecule-1 |
| 14 | Interferon gamma |
| 15 | Interleukin 1 alpha |
| 16 | Interleukin-1 receptor alpha |
| 17 | Interleukin-10 |
| 18 | Interleukin-12 p40 |
| 19 | Interleukin-12 p70 |
| 20 | Interleukin-15 |
| 21 | Interleukin-17 |
| 22 | Interleukin-1beta |
| 23 | Interleukin-2 |
| 24 | Interleukin-23 |
| 25 | Interleukin-3 |
| 26 | Interleukin-4 |
| 27 | Interleukin-5 |
| 28 | Interleukin-6 |
| 29 | Interleukin-7 |
| 30 | Interleukin-8 |
| 31 | Macrophage Inhibitory Protein 1 alpha |
| 32 | Macrophage Inhibitory Protein-1 beta |
| 33 | Matrix metalloproteinase type 2 |
| 34 | Matrix metalloproteinase type 3 |
| 35 | Matrix metalloproteinase type 9 |
| 36 | Monocyte Chemotactic Protein-1 |
| 37 | RANTES |
| 38 | Stem Cell Factor |
| 39 | Tissue Inhibitor of Metalloproteinase |
| 40 | Tumor Necosis Factor beta |
| 41 | Tumor Necrosis Factor alpha |
| 42 | Tumor Necrosis Factor receptor alpha 2 |
| 43 | Vascular Cellular Adhesion Molecule type 1 |
| 44 | Vascular Endothelial Growth Factor |
| 45 | Vitamin D Binding Protein |
| 46 | von Willebrand Factor |

### Iron-Related Measurements.

To measure non-transferrin-bound iron, blood samples will be collected in trace element-free tubes at the defined time points; this will allow for the determination of the kinetics of non-transferrin-bound iron in transfusion recipients (assuming human non-transferrin-bound iron levels increase post-transfusion as in the pre-clinical studies in mice (see FIG. 7)). To measure serum non-transferrin-bound iron, a previously published method [93] will be used, with minor modifications. Briefly, blood samples will be allowed to clot for 20 minutes at room temperature and then centrifuged at 1,000 g at 4° C. for 10 minutes; serum will be decanted and immediately frozen at −80° C. until analysis. To avoid iron uptake in vitro by vacant binding sites on transferrin, serum samples will be treated with tris-carbonatocobaltate (III) trihydrate [94]. An 800 mM nitrilotriacetic acid, pH 7.0 solution (50 μL) will then be added to each 450 μL sample. Following incubation at room temperature for 30 minutes, serum proteins will be removed using an ultracentrifugation filtration device (NanoSep, 30-kDa cutoff, polysulfone type; Pall Life Sciences; 10,620×g for 45 minutes at 10° C.). Finally, iron in the ultrafiltrate will be quantified using a ferrozine assay [95].

Serum hepcidin concentrations may be measured using a recently developed and validated competitive enzyme-linked immunoassay [96]. This assay has excellent intra-assay precision and inter-assay reproducibility and correctly detects the expected physiologic and pathologic variations in hepcidin concentrations. The hepcidin reference ranges using this assay are 29-254 ng/mL for men and 17-286 ng/mL for women, with significantly different medians: 112 vs. 65 ng/mL, respectively. This difference is likely due to the lower iron stores in women. Hepcidin levels exhibit diurnal variation, with noon and evening (i.e. 8:00 PM) values significantly higher than morning (i.e. 8:00 AM) values. Thus, both the “fresh” and “old” transfusions will be scheduled for approximately the same time of day.

Serum iron and total-iron-binding capacity (TIBC) will be measured in the Iron Reference Laboratory in CALM using methods recommended by the International Committee for Standardization in Hematology. The transferrin saturation is calculated as: (serum iron×100)/TIBC.

Plasma hemoglobin and heme will be determined as oxyhemoglobin, methemoglobin, and hemochrome concentrations, as described [97], on blood samples obtained with precautions to avoid inducing hemolysis.

### Other Routine Clinical Laboratory Assays.

As noted above, blood samples will be transported by the staff of CALM to the routine Clinical Laboratories at CUMC. A complete blood count (including hemoglobin, hematocrit, red blood cell count, mean corpuscular volume, white blood cell count with automated white blood cell differential, platelet count, and absolute reticulocyte count) is determined with the XE-5000 Hematology System (Sysmex, Mississauga, ON). Serum concentrations of total bilirubin, direct bilirubin, aspartate aminotransferase, alanine aminotransferase, alkaline phosphatase, albumin, total protein, glucose, blood urea nitrogen, creatinine, lactate dehydrogenase, and ferritin are measured with the AU-2700 Chemistry Analyzer (Olympus, Center Valley, Pa.). Haptoglobin is measured with the BN II Analyzer (Dade Behring Inc., Newark, Del.).

### Statistical Considerations.

In this prospective study of healthy male volunteers, each will be given two RBC transfusions, one control (i.e. “fresh” after 3 days of storage), and one experimental (i.e. “old” after 42 days of storage). The primary study outcome will be a paired comparison for each subject of the maximum difference between each pre- and post-transfusion level of 6 cytokines (Table 1), comparing the “fresh” and “old” RBC transfusions. Two important subsidiary outcomes, comparing pre- and post-transfusion serum non-transferrin-bound iron and hepcidin levels between the “fresh” and “old” RBC transfusions, will be examined.

To provide sample size justification, it is believed that the acute delivery of hemoglobin iron by transfusion of older stored RBCs induces a pro-inflammatory cytokine response. Thus, each participant in the study will receive both a “fresh” and an “old” autologous RBC transfusion separated in time by 39 days. The primary study outcome is a paired comparison for each subject of the maximum concentration difference between a post- and pre-transfusion cytokine level (ΔCₘₐₓ) comparing the “fresh” transfusion on Day #3 and “old” transfusion on Day #42. Therefore, the sample size is estimated only with respect to this primary outcome. Subsidiary analyses will be made with respect to several other study outcomes, but these comparisons are not entered into the sample size calculation.

Based on the pre-clinical data in mice, the infusion of the equivalent of one unit of fresh or older stored RBCs led to a mean ΔCₘₐₓ of plasma MCP-1 (a robust pro-inflammatory cytokine) of 386.4 pg/mL, with a standard deviation of 138.3 pg/mL, when comparing the cytokine levels from transfusing fresh and older stored RBCs. Assuming that a similar difference will be found in humans, then to detect a difference in the ΔCₘₐₓ of plasma MCP-1 levels of at least 150 pg/mL between the fresh Day #3 transfusion and the old Day #42 transfusion (i.e. about 40% of the difference seen in mice), using a paired two-sample t-test with a two-sided significance level of 0.05 and a power of 0.80, approximately 9 subjects will be needed:

$$n = \frac{{( {z}_{a} - {z}_{b} )}^{2} {o}^{2}}{{δ}^{2}} = \frac{{( 1.96 + 1.282 )}^{2} {( 138.3 )}^{2}}{{( 150 )}^{2}} = 8.9$$

In this equation, n indicates the sample size in the study group, zₐ and z_{b} respectively denote the upper α and lower β percent points of the normal distribution, σ is the expected standard deviation, and δ denotes the difference in the ΔCₘₐₓ of the plasma cytokine levels between the old Day #42 and fresh Day #3 transfusions. If about 20% loss of volunteer participants is allowed for during the 45 days of the study (due to inadequate blood sampling, exclusion or withdrawal for any reason, etc.), then 11 subjects will need to be recruited.

Although the subsidiary analyses planned have not entered into the calculation of the sample size required for the study, the indicated sample size should also provide adequate power for the subsidiary comparisons. For example, the pre-clinical data in mice show a ΔCₘₐₓ of non-transferrin-bound iron of 1.4 μM with a standard deviation of 0.6 μM, comparing mice transfused with fresh and older stored RBCs. A sample size of 11 in the human study will provide 80% power to detect a 0.59 μM difference in non-transferrin-bound iron between the fresh Day #3 and older Day #42 transfusions (i.e. about 40% of the difference seen in mice).

Results from Two Healthy Male Volunteers.

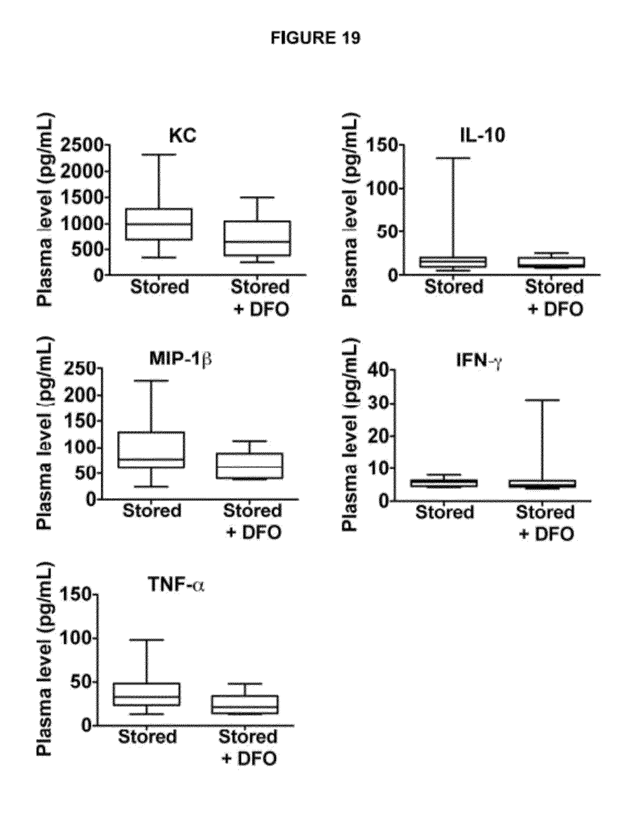

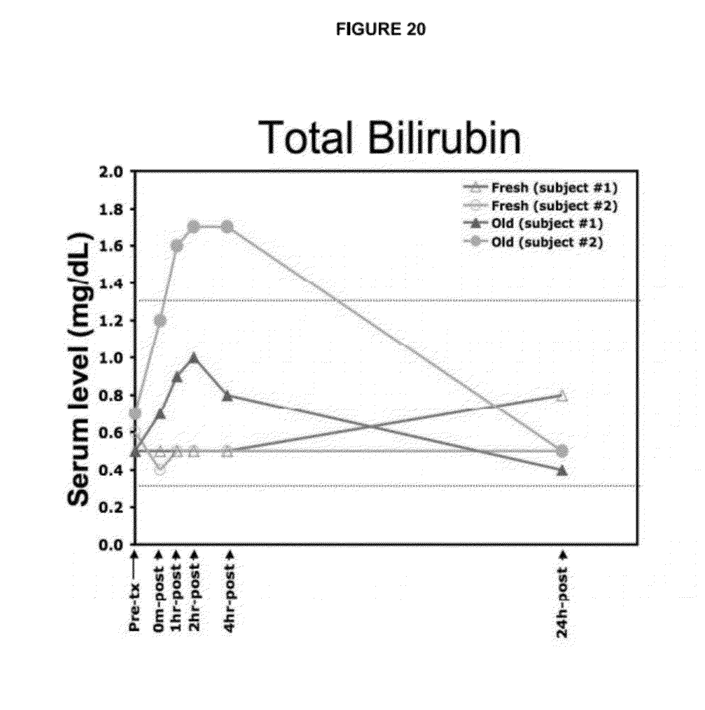

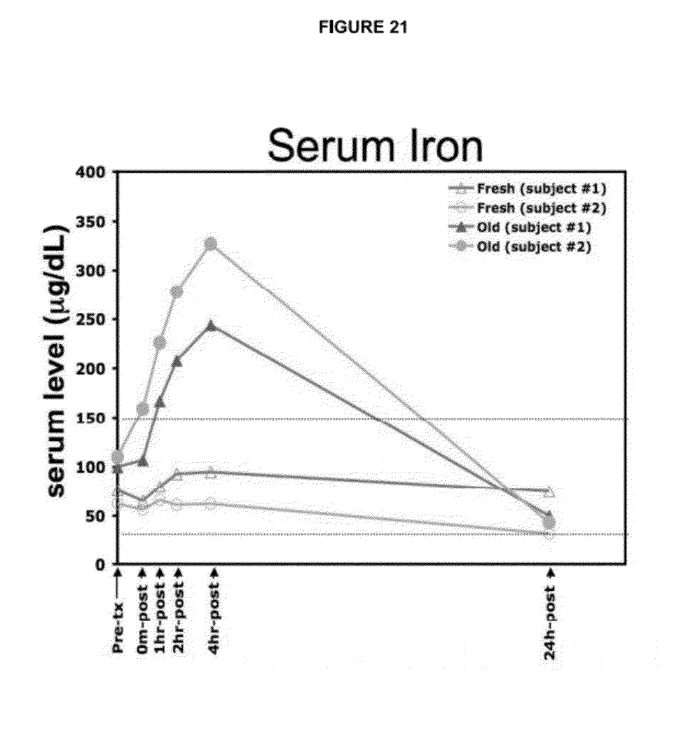

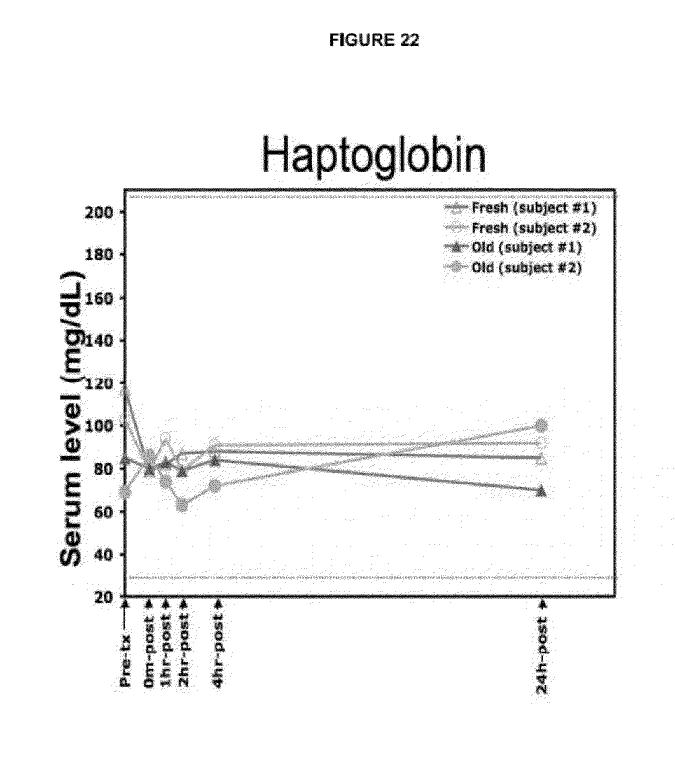

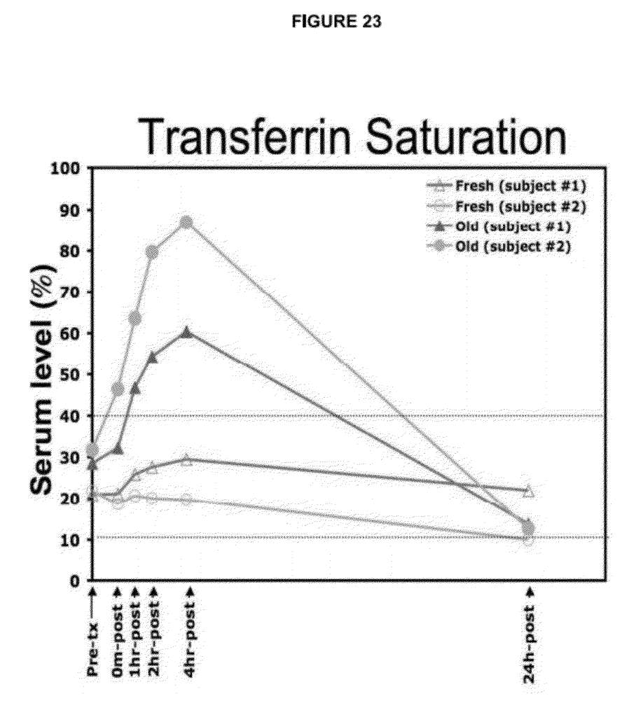

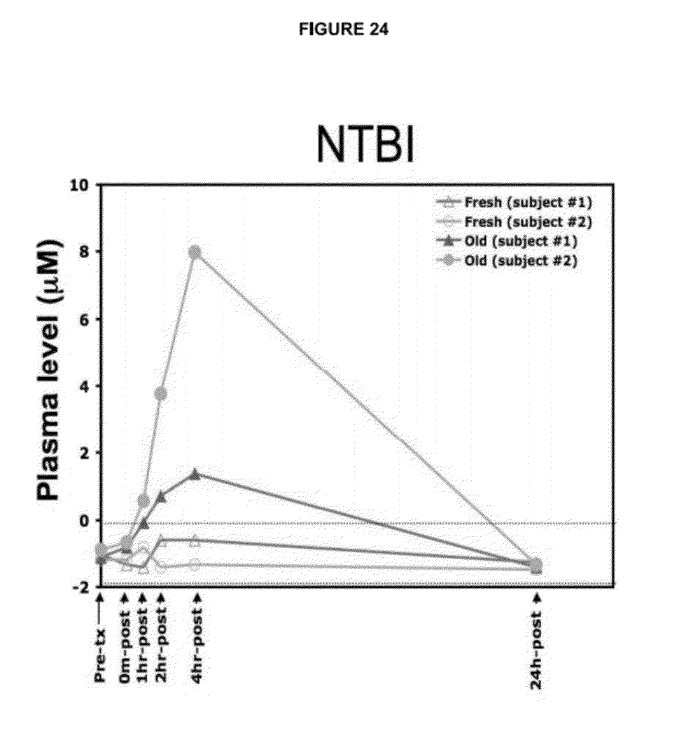

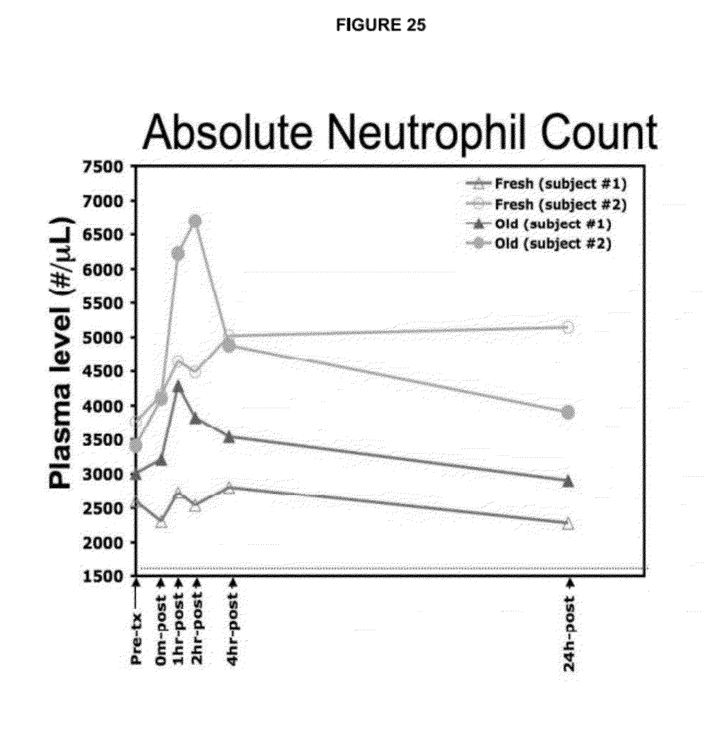

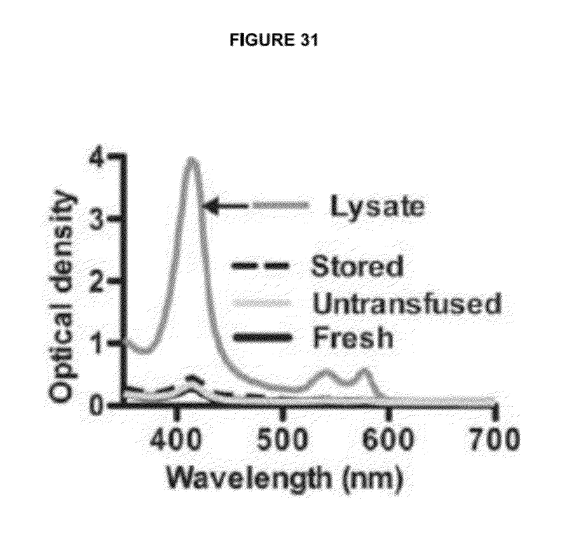

To date, two male volunteers between the ages of 18 and 65 years completed the study and 3 (including 1 female) are currently enrolled. The results from the two subjects are shown in FIGS. 20-26 and 32. Between 0-4 hours after transfusion of only the older stored RBC unit, both volunteers exhibited dramatic increases in total bilirubin, serum iron, transferrin saturation, NTBI, and absolute neutrophil count. In addition, serum hepcidin levels and the pro-inflammatory cytokine, interleukin-6 (IL-6) were elevated in one of the two volunteers (FIG. 32). There is no detectable increase in these analytes after a “fresh” RBC transfusion. There are no detectable changes in haptoglobin levels suggesting that the RBCs are being cleared extravascularly. Taken together, this provides evidence that iron is liberated into the circulation following processing of cleared RBCs; in particular, this clearance is substantial enough to raise the level of plasma NTBI, which is typically undetectable in healthy volunteers. In addition, the acute rise in circulating neutrophils and IL-6 suggests the presence of an inflammatory response following older, stored RBC transfusions.

### Example 6

### Transfusion of Older Stored RBCs into Healthy Individuals—an Expanded Study

Additional experiments involving healthy volunteers were carried out. The details of the experiments are as follows.

### Study Design.

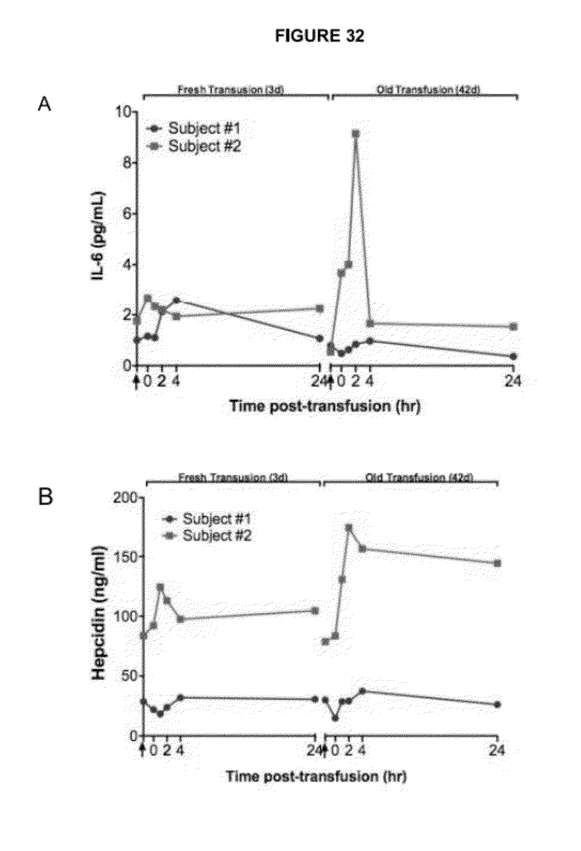

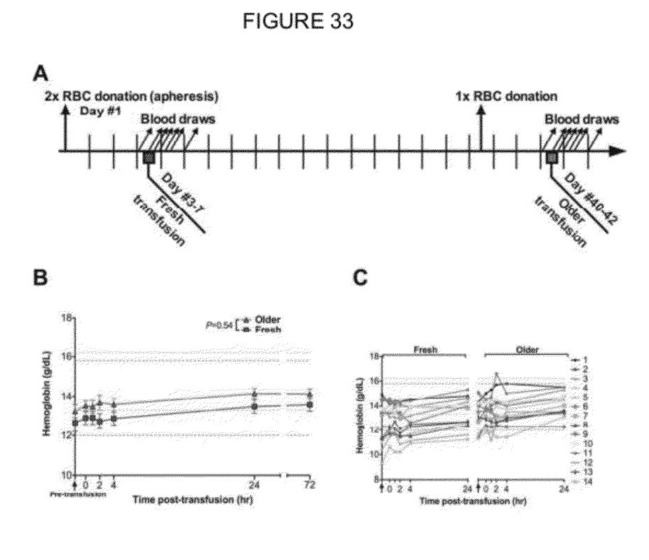

Fourteen healthy adult volunteers were prospectively studied. Each volunteer donated, by a standard automated apheresis method (Alyx, Baxter Healthcare), a leukoreduced, double red blood cell unit, which was stored in a standard additive solution (Adsol/AS-1, Baxter) in the Columbia University Medical Center—New York Presbyterian Hospital Blood Bank in compliance with FDA standards. Each volunteer was then transfused with one autologous red blood cell unit after 3-7 days of storage (i.e., “fresh”) and subsequently with the second unit after 40-42 days of storage (i.e., “older”). Timed blood samples were obtained 90 minutes before each transfusion and at 0-, 1-, 2-, 4-, 24-, and 72-hours after transfusion. To prevent post-transfusion erythrocytosis, and to maintain a parallel study design, a single unit whole blood phlebotomy was performed 3-7 days prior to the transfusion of the older red blood cells, if the volunteer's hemoglobin was >13.3 g/dL (FIG. 33A). In addition, both fresh and older transfusions were started at approximately the same time of day (11:00 a.m.), took place over approximately two hours at a rate of 150 mL/hour, and the same lunch was provided for both transfusion episodes at 12:00 noon. All transfusions were performed at Columbia University Medical Center—New York Presbyterian Hospital and the double red blood cell units were collected and processed at the New York Blood Center. Study recruitment began in December, 2008 and was completed by February, 2011. The research protocol was approved by the Institutional Review Boards of both institutions, was conducted according to the principles expressed in the Declaration of Helsinki, and all participants provided written informed consent. The trial was registered with clinicaltrials.gov with the identifier: NCT01319552.

### Study Participants.

The inclusion criteria were: healthy adults 18-65 years of age with male body weight >59 kg (130 lbs), female body weight >70 kg (155 lbs), male height >1.55 m (5′1″), female height >1.65 m (5′5″), and hemoglobin >13.3 g/dL. Exclusion criteria were: ineligibility for donation based on the New York Blood Center autologous blood donor questionnaire, systolic blood pressure >180 or <90 mm Hg, diastolic blood pressure >100 or <50 mm Hg, heart rate <50 or >100, temperature >37.5° C. prior to donation, temperature >38° C. or subjective feeling of illness prior to transfusion, positive results on standard blood donor infectious disease testing, and pregnancy. All screened volunteers who met the inclusion criteria, and did not meet any of the exclusion criteria, were enrolled in the study.

### Laboratory Measurements.

All laboratory testing for routine clinical parameters was performed in the Columbia University Medical Center—New York Presbyterian Hospital Clinical Laboratories. Nontransferrin-bound iron was measured using an ultrafiltration assay, as described [143], and was performed in the Iron Reference Laboratory at the Columbia University Medical Center. The reference range for plasma non-transferrin-bound iron in the laboratory is −0.71 to 0.10 μM. Data are presented as a change in non-transferrin-bound iron from pretransfusion levels. To eliminate interassay variability biasing the change in nontransferrin-bound iron levels, all the samples for a given volunteer were frozen at −80° C. and were analyzed together following the final time-point of study participation.

Interleukin (IL)-6 was measured with a high sensitivity ELISA kit (R&D Systems) following the manufacturer's instructions.

### Bacterial Proliferation In Vitro.

Proliferation of a pathogenic strain of E. coli, obtained from an anonymous patient with a urinary tract infection, was measured after inoculating all serum samples obtained from study participants, both before and after transfusion, as described [143]. Briefly, 100 μL aliquots of serum in microtiter plate wells were incubated with 1×10⁶ colony forming units of E. coli at 37° C. with shaking. Optical density at 600 nm was measured periodically up to 5 hours after inoculation using a PowerWave XS microtiter plate reader (BioTek) and the area under the curve of the resultant growth curve was calculated using Prism 5 (GraphPad Software, Inc.). All samples were inoculated in duplicate and the mean of the two growth curves was used.

### Statistical Analysis.

Differences in outcome measures after fresh or older red blood cell transfusions were compared using a paired t-test or a Wilcoxon matched pairs test, as appropriate, to compare the area under the curve of the increase in the outcome measure from 0- to 24-hours after transfusion. Normality of data was assessed using a D'Agostino and Pearson omnibus normality test. A P value of less than 0.05 was considered significant. Statistical analyses were performed using Prism 5. All data are presented as mean±SEM, unless otherwise specified. The study was originally powered for 11 participants, a sample size appropriate for detecting 15% of the non-transferrin-bound iron difference seen in mice, assuming the same standard deviations as observed in the mouse model. Recruitment was increased to 14 volunteers to include additional female participants, who were underrepresented in the first 11 volunteers. Exclusion of these additional female volunteers from the analysis did not significantly change the results.

Results Subject characteristics.

Of 42 consecutive adults screened, 14 qualified for participation and all 14 volunteers completed the study with no dropout (study design shown in FIG. 33A). There were no transfusion reactions and no significant changes in vital signs (blood pressure, heart rate, temperature) throughout the study. Two volunteers experienced lightheadedness during or immediately after donating blood and one volunteer, who suffers from chronic migraines, experienced a headache and vomited 2 hours after transfusion of fresh red blood cells; the latter event was considered to be unrelated to study participation by both the study participant and the Data Safety Monitoring Board. Because of lower baseline hemoglobin levels (Table 3), only one of the four female participants met the hemoglobin criterion (>13.3 g/dL) for the one unit whole blood phlebotomy 3-7 days before the older red blood cell transfusion. Exclusion of the three non-phlebotomized female participants from the analyses did not significantly change the results; thus, they were included in the analysis. All male participants met the hemoglobin criterion and were phlebotomized one whole blood unit before the older red blood cell transfusion.

TABLE 3
Baseline Characteristics of Volunteers

| | Volunteers (N = 14) |
|---|---|
| Characteristic | |
| Age - year (mean ± SD) | 30.4 ± 9.1 |
| Female gender - no. (%) | 4 (28.6) |
| Race/ethnicity - no. (%)* | |
| White | 9 (64) |
| Black | 1 (7) |
| Asian | 2 (14) |
| Hispanic | 2 (14) |
| ABO type - no. (%) | |
| A | 6 (43) |
| B | 5 (36) |
| O | 3 (21) |
| AB | 0 (0) |
| Height - meters (mean ± SD) | 1.79 ± 0.09 |
| Weight - kg (mean ± SD) | 87.5 ± 17 |
| Hemoglobin pre-study - g/dL (mean ± SD) | |
| Male | 15.3 ± 1.2 |
| Female | 14.2 ± 0.8 |

*Race/ethnicity was assessed by the investigators

### Effect of Storage Duration on Complete Blood Counts.

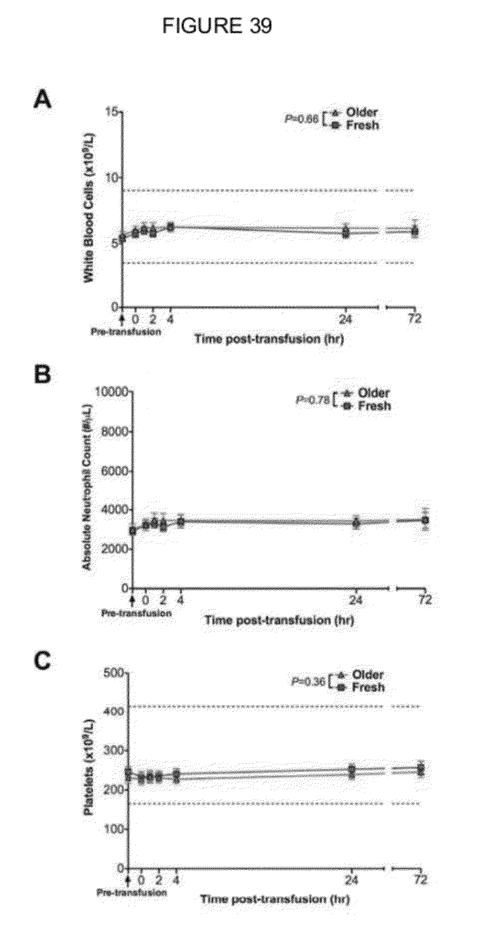

The mean pre-transfusion hemoglobin (i.e. after donating the red blood cell units, but 90 minutes before transfusion) was 12.7 and 13.3 g/dL, for the fresh and older red blood cell transfusions, respectively (P=0.07 by paired t-test). Given the volume of blood drawn for the timed samples, the hemoglobin was expected to increase by about 0.8 g/dL following transfusion. At 4-hour post-transfusion, the mean hemoglobin increased by only 0.22 g/dL and 0.34 g/dL for the fresh and older transfusions, respectively. However, by 24-hour post-transfusion, the mean hemoglobin increased by 0.82 and 0.89 g/dL for the fresh and older transfusions, respectively (P=0.5442; FIGS. 33B-C). No significant differences between fresh and older transfusions were observed for white blood cell, absolute neutrophil, or platelet counts (FIG. 39).

### Effect of Storage Duration on Basic Metabolic Parameters.

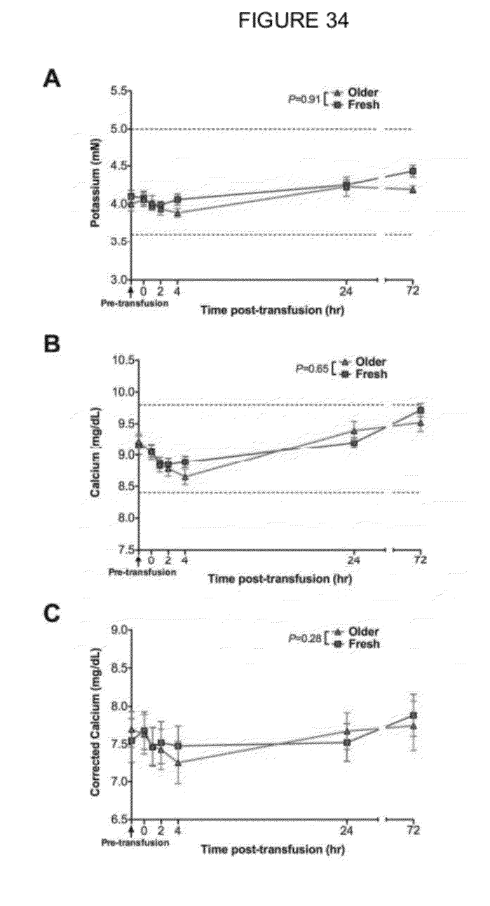

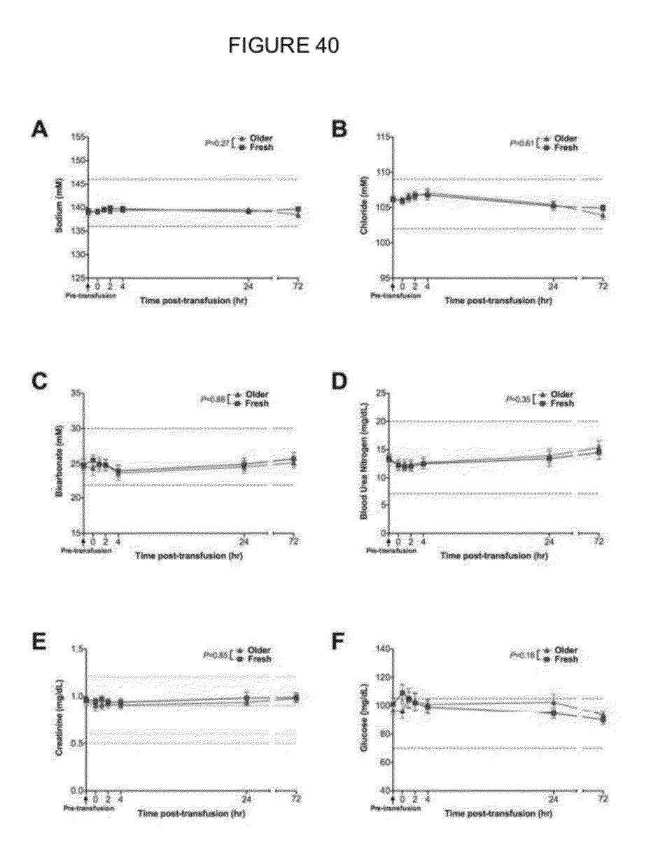

There were no significant differences between fresh and older red blood cell transfusions in basic metabolic parameters (i.e. sodium, potassium, chloride, blood urea nitrogen, creatinine, glucose, and total calcium; FIGS. 34 and 40). In particular, increased potassium levels were not observed following the older red blood cell transfusions (FIG. 34A). However, both types of transfusions were associated with a progressive decrease in total calcium up to 4 hours after transfusion (FIG. 34B), which was still evident following older red blood cell transfusions when the calcium levels were corrected for serum albumin levels (FIG. 34C); ionized calcium was not measured.

### Transfusion of Older Red Blood Cells Results in Extravascular Hemolvsis.

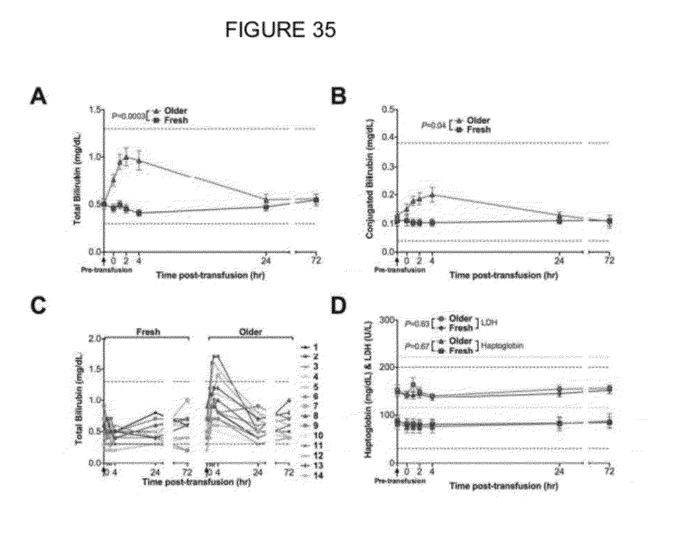

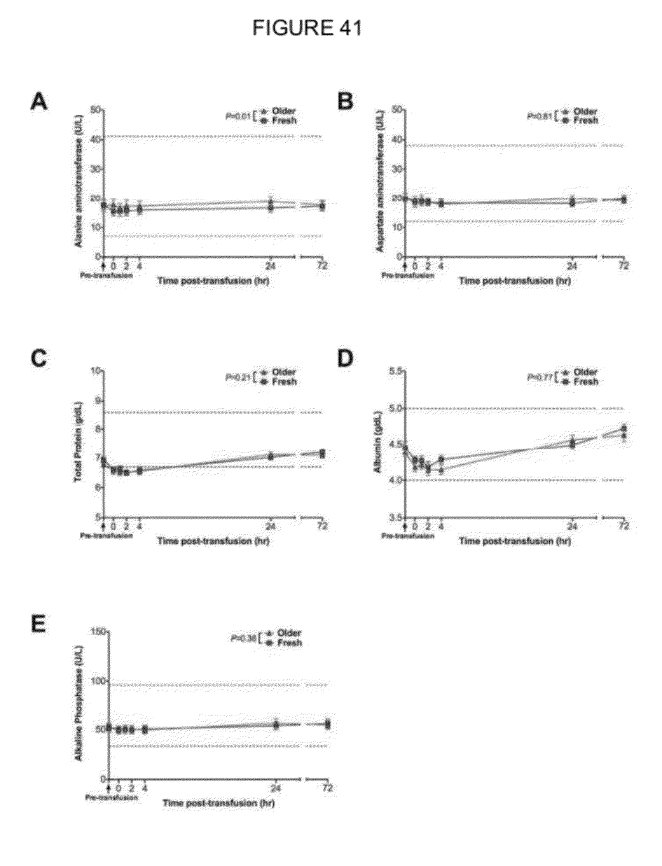

As compared to transfusions of fresh red blood cells, transfusions of older red blood cells were associated with significantly increased serum unconjugated bilirubin (P=0.0002; FIG. 35A), with a mean peak increase in unconjugated bilirubin of 0.55 mg/dL at 4 hours after transfusion. In addition, unconjugated bilirubin peaked above the reference range in 3 of 14 volunteers after transfusion of older red blood cells (FIG. 35C). Although the bilirubin was predominantly unconjugated, there was a small, but significant, rise in serum conjugated bilirubin (FIG. 35B). No statistically significant differences between fresh and older red blood cell transfusions were observed in mean serum haptoglobin and lactate dehydrogenase levels, which are indicators of intravascular hemolysis (FIG. 35D). There was a significant difference between transfusions of fresh and older red blood cells in alanine aminotransferase levels (FIG. 41; P=0.01) although the difference was small (a 1.7 U/L increase 4 hours following older transfusions as compared to fresh transfusions). There were no significant differences between fresh and older red blood cell transfusions in the other tested liver function parameters (i.e. aspartate aminotransferase, alkaline phosphatase, total protein, and albumin; FIG. 41).

### Transfusions of Older Red Blood Cells Increase Iron Parameters and Produce Circulating Non-Transferrin-Bound Iron.

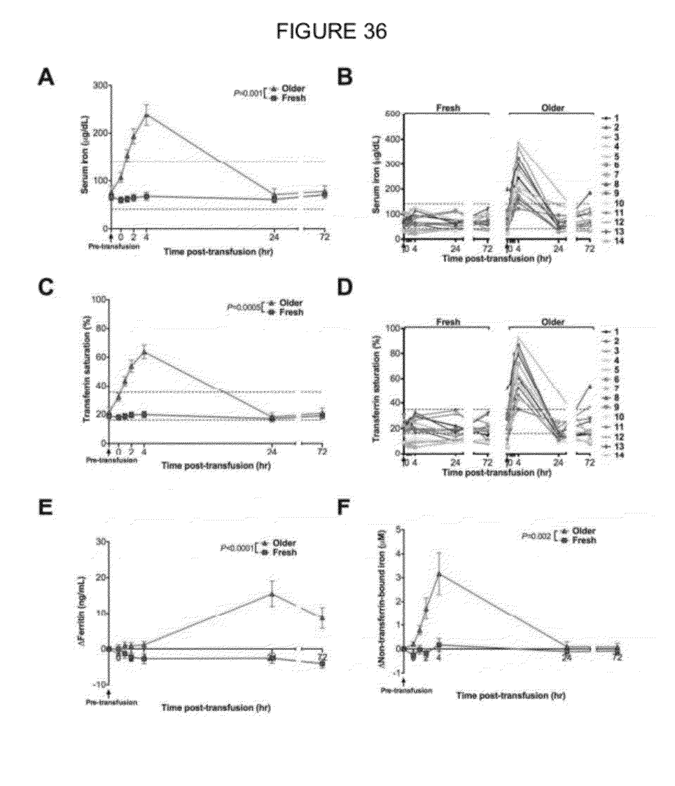

Although transfusions of fresh red blood cells produced no significant change in mean serum iron or transferrin saturation, transfusions of older red blood cells led to significant increases in serum iron (P=0.001) and transferrin saturation (P=0.0005), with a mean increase of 162 μg/dL and 42% over baseline, respectively, at 4 hours after transfusion (FIGS. 36A and C). In particular, serum iron and transferrin saturation peaked above the reference range in 13 of 14 volunteers after transfusion of older red blood cells (FIGS. 36B and D). In addition, ferritin levels increased from the baseline pre-transfusion sample only after transfusion of older red blood cells, peaking at 15.5 ng/mL above baseline at 24 hours after transfusion (FIG. 36E). Furthermore, after the fresh red blood cell transfusions, no significant increases in circulating non-transferrin-bound iron concentration were observed. In contrast, 13 of 14 volunteers had progressively increasing circulating non-transferrin-bound iron between 1 to 4 hours after transfusion of older red blood cells reaching a mean of 3.2 μM (P=0.002) over baseline at 4 hours post-transfusion (FIG. 36F).

### Effect of Red Blood Cell Storage Duration on Inflammation.

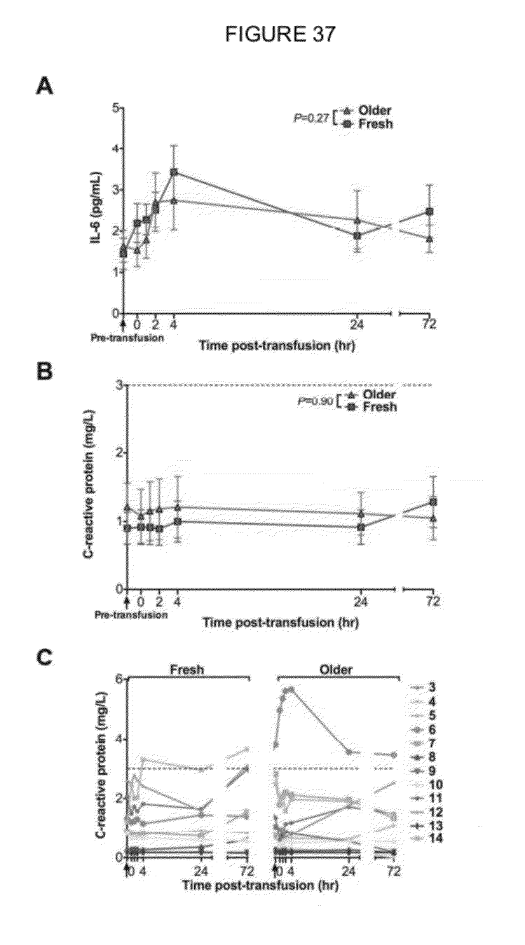

Prior studies in mice demonstrated increases in various markers of inflammation after transfusions of older red blood cells [143]. However, in the current human study, there were no significant differences in IL-6 or C-reactive protein (CRP) levels between the groups receiving fresh and older red blood cell transfusions (FIGS. 37A and B). Nonetheless, one volunteer (number 14), had a CRP rise above the reference range between 4 and 72 hours after transfusion of only the fresh red blood cells and one volunteer (number 6), who had an elevated CRP level prior to the older red blood cell transfusion, manifested a progressive rise in CRP peaking 4 hours after transfusion of only the older red blood cells (FIG. 5C). Interestingly, in a post-hoc analysis, this volunteer was African-American with a Duffy-negative red blood cell antigen phenotype. Because the Duffy antigen is a chemokine receptor rarely absent on red blood cells of individuals of non-African descent, none of the other volunteers' red blood cells were tested for the Duffy phenotype.

### Transfusions of Older Red Blood Cells Enhance Bacterial Growth In Vitro.

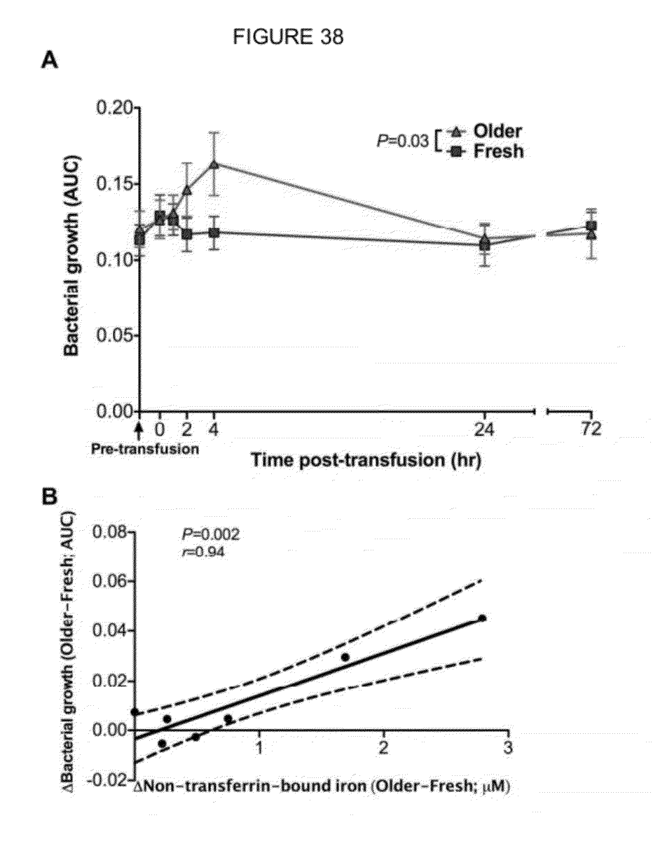

A pathogenic strain of E. coli obtained from an anonymous patient with a urinary tract infection was inoculated into all serum samples from all volunteers at all time points surrounding each transfusion. At 2 to 4 hours after transfusion of older (P=0.03, FIG. 38A), but not fresh, red blood cells, the growth of E. coli was enhanced in these serum samples. The mean difference in the area under the growth curve between fresh and older red blood cell transfusions correlated with the mean change in non-transferrin bound iron (P=0.002, Pearson r=0.94; FIG. 38B). Prior studies confirmed the iron-dependent growth of this bacterial isolate [143].

These results provide evidence of physiological differences in the consequences of transfusions of red blood cells after shorter (3-7 days) or longer (40-42 days) durations of storage, despite strict adherence to current FDA standards. Transfusions of fresh red blood cells to 14 healthy volunteers produced no detected laboratory evidence of hemolysis and did not significantly alter serum iron, transferrin saturation, or circulating non-transferrin-bound iron. In contrast, despite appropriate increases in hemoglobin level, transfusions of older red blood cells led to increased mean serum unconjugated bilirubin levels with no significant changes in mean serum haptoglobin or lactate dehyrodgenase levels, a pattern consistent with rapid extravascular hemolysis of a subpopulation of the transfused older red blood cells. Importantly, during the initial 4 hours after transfusion of older red blood cells, serum iron and transferrin saturation increased significantly and circulating non-transferrin-bound iron appeared. These changes returned to baseline by 24 hours after transfusion. The potential pathogenic import of these differences was shown using a bacterial growth assay with these serum samples: increased proliferation in vitro of a pathogenic strain of E. coli correlated with increased concentrations of non-transferrin-bound iron (r=0.94, P=0.002). Although no untoward clinical events occurred in these healthy volunteer recipients of older transfused red blood cells, the potential for adverse infectious outcomes is conceivable, particularly for patients after cardiac surgery or trauma, who have open entry points for bacterial invasion and may be rapidly transfused with multiple units of red blood cells of varying storage duration. However, further studies are necessary to confirm these findings in the clinical setting in vivo.

As red blood cells age, while circulating in vivo or stored in vitro, they undergo changes that eventually lead to their recognition as senescent or damaged, and to their removal by macrophages in the spleen, bone marrow, and liver [143, 145]. In a typical healthy adult, approximately 1 mL of red blood cells reach the end of their life span and are cleared each hour, yielding about 1 mg of iron. This iron is either stored intracellularly or returned to the plasma to be bound by transferrin and transported to the erythroid marrow and other tissues for re-use. By current FDA standards, a unit of stored red blood cells is acceptable for transfusion even if 25% of the red cells are cleared within 24 hours, an amount equivalent to as much as 60 mg of iron. Because most of this clearance takes place during the first hour after transfusion [33], the rate of delivery of heme-iron to reticuloendothelial macrophages may abruptly increase by as much as 60-fold after transfusion of even a single unit of packed red blood cells. The corresponding accelerated rate of return of iron to plasma can surpass the rate of uptake by transferrin and produce circulating non-transferrin-bound iron.

In this Example, the kinetics of the appearance in the circulation of red blood cell degradation products (e.g. bilirubin) demonstrate the rapid extravascular clearance of a sub-population of the older transfused red blood cells. Thus, although the mean hemoglobin increment following transfusion did not significantly differ between fresh and older red blood cell transfusions, the serum unconjugated bilirubin and transferrin saturation levels increased rapidly and in parallel during the initial 4 hours after transfusion of older stored red blood cells (FIGS. 35 and 36). Despite the continued presence of unsaturated transferrin, circulating non-transferrin-bound iron appeared, probably because the rate of iron influx into plasma overwhelmed the rate of iron acquisition by plasma transferring [146]. Because plasma non-transferrin-bound iron, a heterogeneous assortment of iron complexes [55, 147], is available to pathogens reaching the blood stream and can enhance their growth [148, 149], proliferation in vitro of a pathogenic strain of E. coli was examined. As shown in FIG. 38A, serum samples obtained after transfusion of older red blood cells significantly enhanced bacterial growth. Furthermore, as shown in FIG. 38B, increased bacterial growth in serum obtained after transfusion of older, as compared to fresher, red blood cells closely correlated with the corresponding increases in circulating non-transferrin-bound iron. Because bacteria use various mechanisms for procuring iron [150], the contribution of non-transferrin-bound iron to virulence is expected to vary among organisms. Nonetheless, withholding iron from pathogens is a central component of host defense [149], and our results in vitro illustrate the capacity of circulating non-transferrin-bound iron to enhance infection. Interestingly, patients with transfusional iron overload, as well as those with hereditary forms of hemochromatosis, may have circulating non-transferrin-bound iron levels [151, 152] similar to those measured in our healthy volunteers at 4 hours after transfusion with older red blood cells and are known to be at an increased risk for acute and chronic infections with specific pathogens [153-155]. In addition, oral iron supplements are associated with transient increases in non-transferrin-bound iron [146, 156] and routine supplementation with iron and folic acid in children in a malaria-endemic region increased the risk of severe illness and death [157, 158]. An incidental finding from this study is the extent of variability in hemoglobin levels measured soon after transfusion (FIGS. 33B and C), with 6/14 volunteers exhibiting either a decrease or no increase in hemoglobin at 4-hours after transfusion of fresh autologous red blood cells. Although this finding supports classic textbook teaching [159] that it requires up to 24 hours for hemoglobin levels to equilibrate, more recent studies suggest that hemoglobin levels quickly equilibrate after transfusion in adult [160] and neonatal [161] patients.

Therefore, these findings suggest that hemoglobin measurements may not be ideal for assessing the effectiveness of red blood cell transfusions until 24 hours after transfusion, although this is assertion is limited by an absence of measurements between 4 and 24 hours after transfusion. Decreases in serum albumin and total protein levels following transfusion of both fresh and older red blood cells (FIG. 41) suggest that there are significant volume shifts from the extravascular to the intravascular space following transfusion, which may help explain the observed variability in hemoglobin levels.

Unlike the results in mouse studies, no difference in pro-inflammatory IL-6 levels were observed in healthy human volunteers following transfusion of fresh or older red blood cells. Although mice may handle transfusion-induced iron loads differently than humans, the red blood cell dose transfused into humans may also have been too small and may have been given too slowly to elicit a pro-inflammatory cytokine response. For example, in the mouse studies, the rapid infusion (i.e. “IV push”) of two red blood cell units elicited a robust pro-inflammatory cytokine response; in contrast, the human volunteers were transfused with only one red blood cell unit over a 2 hour time period.

Several limitations of this study should be taken into consideration. For example, only one unit was transfused over two hours per transfusion event; therefore, these results likely underestimate the effect on markers of hemolysis following transfusion in hospitalized patients who frequently receive multiple units at faster infusion rates. In addition, no blood samples were drawn between 4 and 24 hours after transfusion; therefore, the actual time intervals during which the iron parameters and markers of hemolysis remain elevated following transfusion of older red cells are unknown. Still, these parameters predominantly return to baseline by 24 hours after transfusion, thereby indicating a relatively transient effect. Further studies are necessary to determine whether these transient effects are significant enough to affect the clinical course of transfused patients. Finally, there is an inherent time bias in the study design in that the older red blood cells were always transfused about 35 days after the fresh blood transfusions.

These studies in healthy human volunteers, and the related investigations in mice [143, 162], demonstrate that increased transferrin saturation leading to production of circulating non-transferrin-bound iron after transfusion of older red blood cells is a potential mechanism for enhancing infectious complications in recipients. The concentrations of non-transferrin-bound iron observed after slow transfusion of a single unit of autologous red blood cells in healthy human volunteers may be considerably lower than those found after rapid transfusion of multiple sequential units of allogeneic red blood cells to severely ill trauma and surgical patients. Circulating non-transferrin-bound iron can also produce oxidative damage, thrombosis, cytotoxicity, and other types of injury [143, 55, 125, 163], and may contribute to additional mechanisms of increased morbidity and mortality after transfusions of older red blood cells. Finally, other proposed mechanisms (e.g. involving nitric oxide and/or microvesicles) may contribute to the increased morbidity and mortality that may result from transfusions of older red blood cells [142, 25, 164-166].

### Example 7

### Transfusion of Older Stored RBCs Induces an Acute Pro-Inflammatory Response in Chronically Transfused Patients with Sickle Cell Disease or β-Thalassemia

It is believed that transfusion of older, stored blood induces a pro-inflammatory cytokine response in patients with sickle cell disease and β-thalassemia. In addition, whether other standard RBC products that are often used in this setting (i.e. washed RBCs and cryopreserved RBCs) induce similar effects will determined. Interestingly, storage of human donor RBC units in vitro leads to progressive increases in non-transferrin-bound iron levels, presumably by iron derived from damaged or hemolyzed RBCs, which eventually saturate the available transferrin in the supernatant. In addition, RBC transfusions, at least in infants, lead to increased levels of plasma non-transferrin-bound iron [59]. If concomitant infusion of non-transferrin-bound iron by transfusion of older stored RBC units is harmful, then iron chelators and anti-oxidants may prevent this damage [31, 60]. Similarly, washing older stored RBC units should remove the non-transferrin-bound iron in the supernatant, thereby preventing its infusion. However, because it is believed that washing RBCs does not remove the toxic component of the transfusion (because the toxic component is the aged RBCs themselves), the subsidiary belief that washed RBCs still cause a pro-inflammatory cytokine response will be tested. In the pre-clinical studies in mice, washing the older stored RBCs did not prevent induction of a pro-inflammatory state post-transfusion (FIG. 6); this example will address this issue in human patients by washing older stored RBC units immediately pre-transfusion. Also, because the cryopreservation process may damage RBCs, the subsidiary belief that cryopreserved RBCs induce an even greater pro-inflammatory cytokine response will be tested. In this study, only those units that are cryopreserved soon after donation will be used. Finally, by comparing the effects of older stored RBC transfusions in patients with an ongoing chronic hemolytic condition (i.e. sickle cell disease) to patients with a lesser degree of ongoing hemolysis (i.e. β-thalassemia), the subsidiary belief that chronic hemolysis diminishes the pro-inflammatory response will be tested. Chronic hemolysis may decrease or prevent the pro-inflammatory response because of up-regulation of iron protective genes (e.g. heme oxygenase-1) in these patients. The study described below is designed to provide the data to test these beliefs systematically.

### Overview.

A cohort of patients with sickle cell disease or β-thalassemia who receive chronic simple transfusions every 2-6 weeks, who do not have any detectable RBC alloantibodies, and who participated in prior research studies while being treated at CUMC was identified [90, 91]. (Table 4). Their average age is 18.8 years (range 4-42) with 65% males. A review of their transfusion history over the past 6 months shows that the storage time of the units transfused to these patients ranges from 5-32 days, and most patients are transfused with 1-2 units every 2-6 weeks. All of the β-thalassemia patients have had a splenectomy and the sickle cell disease patients are presumed to be functionally a splenic; therefore, most RBC clearance in these patients will likely occur in the liver.

TABLE 4
Characteristics of likely participants in the study.

| | | | | | | | | Age of | | |
|---|---|---|---|---|---|---|---|---|---|---|
| | | | | | | | | units | | |
| | | | | ABO | # units/ | | | (range in | | % |
| No | Disease | Age | Gender | type | event | Frequency | | days) | Hb | Retic |
| 1 | Sickle cell | 19 | F | O+ | 2 | 3-4 | weeks | 7-27 | 8.9 | 20.1 |
| 2 | Sickle cell | 19 | M | O+ | 2 | 3-4 | weeks | 7-26 | 9.5 | 16 |
| 3 | Sickle cell | 19 | F | O+ | 2 | 4 | weeks | 10-26 | 10.2 | 6.8 |
| 4 | Sickle cell | 14 | F | AB+ | 2 | 3-5 | weeks | 9-28 | 9.1 | 9.8 |
| 5 | Sickle cell | 15 | M | B+ | 2 | 3-4 | weeks | 8-28 | 7.6 | 19.8 |
| 6 | Sickle cell | 18 | F | B− | 2 | 4 | weeks | 14-28 | 9.1 | 4.2 |
| 7 | Sickle cell | 14 | F | A+ | 1 | 4 | weeks | 7-24 | 8 | 7.4 |
| 8 | Sickle cell | 8 | M | B+ | 1 | 2-4 | weeks | 5-28 | 8.6 | 11.9 |
| 9 | Sickle cell | 16 | M | O+ | 2 | 4-6 | weeks | 5-17 | 10 | 12.9 |
| 10 | Sickle cell | 4 | M | O+ | 1 | 3-4 | weeks | 10-27 | 9.7 | 15.9 |
| 11 | Sickle cell | 19 | M | A+ | 1 | 3-4 | weeks | 7-18 | 9.9 | 7.4 |
| 12 | Sickle cell | 13 | M | B+ | 1 | 3-4 | weeks | 8-32 | 9.7 | 5.4 |
| 13 | Sickle cell | 20 | M | O− | 2 | 3 | weeks | 8-30 | 11.9 | 15.9 |
| 14 | Sickle cell | 15 | M | O+ | 1 | 4 | weeks | 6-19 | 9.3 | 10.8 |
| 15 | Sickle cell | 6 | F | A+ | 1 | 2-3 | weeks | 9-27 | 9.6 | 6.1 |
| 16 | Sickle cell | 17 | M | A+ | 2 | 4 | weeks | 5-22 | 11.9 | 15.8 |
| 17 | Sickle cell | 13 | M | A+ | 2 | 3-5 | weeks | 14-30 | 10 | 17.6 |
| 18 | Sickle cell | 11 | F | A+ | 2 | 4-5 | weeks | 6-19 | 9.3 | 10.3 |
| 19 | Sickle cell | 16 | F | O+ | 2 | 3-6 | weeks | 6-25 | 8.9 | 8.5 |
| 1 | β- | 24 | M | O+ | 2 | 3-5 | weeks | 6-24 | 10.7 | 4.5 |
| | thalassemia | | | | | | | | | |
| 2 | β- | 25 | M | O+ | 2 | 3 | weeks | 6-25 | 9.9 | 0.3 |
| | thalassemia | | | | | | | | | |
| 3 | β- | 23 | F | O+ | 2 | 3-4 | weeks | 6-27 | 11.1 | 1.7 |
| | thalassemia | | | | | | | | | |
| 4 | β- | 30 | M | A+ | 2-3 | 3 | weeks | 11-30 | 10 | 0.3 |
| | thalassemia | | | | | | | | | |
| 5 | β- | 37 | M | B+ | 2 | 3 | weeks | 8-28 | 10 | 1.2 |
| | thalassemia | | | | | | | | | |
| 6 | β- | 42 | F | A− | 2 | 4-5 | weeks | 9-28 | 11.1 | 0.5 |
| | thalassemia | | | | | | | | | |
| 7 | β- | 33 | M | B+ | 2 | 3 | weeks | 17-29** | 10.4 | 0.8 |
| | thalassemia | | | | | | | | | |
| 8 | β- | 26 | F | A+ | 2 | 2-3 | weeks | 8-30 | 9.5 | 1.3 |
| | thalassemia | | | | | | | | | |
| 9 | β- | 10 | M | O+ | 1 | 3 | weeks | 6-24 | 9.4 | 2.4 |
| | thalassemia | | | | | | | | | |

**indicates that this patient received washed RBCs over the past 6 months, although this is not clinically required and will not interfere with participation in this study.

In Table 4 above, the number of units transfused per event, the frequency of transfusions, and the storage age of units transfused were calculated based on a review of the transfusion history over the past 6 months. The hemoglobin (Hb in g/dL) and % reticulocyte count (% retic) are pre-transfusion values taken immediately prior to their most recent transfusion.

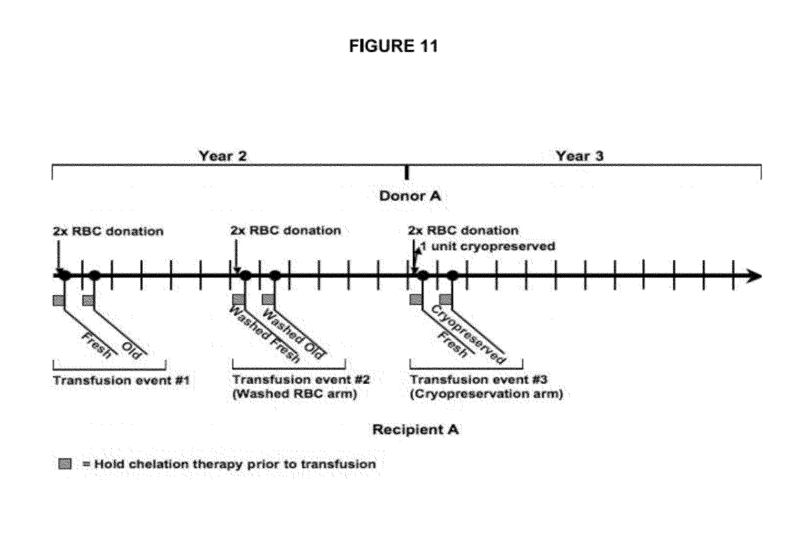

To provide these chronically transfused patients with some benefit from participation, and to control for blood donor variability, dedicated donors will be recruited to supply the 6 transfusions per recipient. Recruiting dedicated donors will reduce the exposure risk to these chronically transfused patients and will be managed by the New York Blood Center. IRB approval for the studies will be obtained. In brief, as shown in FIG. 11, there will be 3 paired transfusion events for which the same donor will provide a double RBC unit by apheresis in each instance, which will be leukoreduced and stored in standard AS-1 preservative. Each of the two units from a given donation will be transfused in sequence, 3-6 weeks apart, into the same hemoglobinopathy patient. There is reasonable flexibility in each patient's transfusion schedule; thus, for this study, a “fresh” transfusion will be defined as between 3-14 days of storage and an “old” transfusion will be defined as between 28-42 days of storage. The first paired transfusion event will examine whether there is a detectable pro-inflammatory cytokine response when comparing “fresh” and “old” RBC transfusions. The second paired transfusion event will examine if there is a beneficial, or adverse, effect of washing RBCs prior to transfusion. The third paired transfusion event will examine the effect of RBC cryopreservation on the pro-inflammatory response.

To quality control the RBC transfusions, the survival in vivo of the transfused RBCs will be quantified. Little is known about RBC survival of stored RBC transfusions (including the effect of washing and cryopreservation) in sickle cell disease and β-thalassemia patients. Therefore, performing RBC survival studies will confirm that a negative primary outcome for this study (i.e. the lack of a pro-inflammatory cytokine response) is indeed due to a difference in pathophysiological mechanism, rather than failure of older stored RBCs to be cleared. To this end, a 10 ml aliquot of donor RBCs will be biotin labeled [98], and survival will be calculated by flow cytometric detection of circulating biotin-labeled RBCs in subsequent blood draws (details provided below). Blood samples (1 ml in EDTA) for calculating RBC survival will be obtained 5 minutes and 1 hour after infusion of 5 ml of biotin-labeled RBCs. The remainder of the RBC unit will be transfused after collection of the 1-hour sample. Because this aspect of the study may limit patient recruitment, participation in this component will be optional.

Blood samples for other analyses (5-10 mL depending on estimated patient total blood volume) will be drawn pre-transfusion, and 1 and 2 hours post-transfusion. These time points were selected based on the pre-clinical mouse studies; however, they may need to be adjusted after examining the results from Example 4. Table 5 summarizes the analytes that will be measured at each time point; these are focused on markers of inflammation (e.g. cytokines), hemolysis (e.g. haptoglobin, free hemoglobin), iron-related measures, and relevant physiological systems (e.g. evaluating renal function using creatinine and blood urea nitrogen). When limited by blood sample volume, priority will be given to laboratory tests higher in the table. Cytokines will also be measured in the RBC units pre-transfusion to determine whether levels detected in recipients are due to endogenous production in vivo. In addition, non-transferrin-bound iron will be measured in the RBC units to test whether washing RBCs decreases the accumulation of this potentially harmful substance in the stored unit.

TABLE 5
Circulating analytes to be measured at each time point

| Category | Analytes measured |
|---|---|
| Cytokines | MCP-1, IL-6, IL-8, IL-10, TNF-α, IFN-γ |
| Iron-related | Non-transferrin-bound iron, hepcidin, serum iron, total iron- |
| | binding capacity (i.e. serum transferrin saturation), ferritin, |
| | free hemoglobin and heme |
| Hemolysis | Lactate dehydrogenase, haptoglobin, direct and indirect |
| | bilirubin |
| Other | Complete blood count with absolute reticulocyte count and |
| | differential count, chemistry panel (e.g. blood urea |
| | nitrogen, creatinine, etc.) |
| Optional | RBC survival study: biotin-labeled RBC detection by flow |
| | cytometry |

If a patient is regularly transfused with more than one RBC unit in each instance based on his transfusion regimen, then the directed unit will be transfused first and the post-transfusion blood samples will be drawn during the subsequent non-directed transfusion. To prevent subsequent transfusions from interfering with cytokine results, the subsequent random donor transfusions during these instances will be fresh (i.e. <14 days of storage). Finally, because all of these patients receive chronic iron chelation therapy, and because the pre-clinical studies suggest that this may affect study outcome, the patients receiving deferoxamine (Desferal, Novartis, t_{1/2}=6 hours) will stop chelation therapy 36 hours prior to each of the 6 planned study transfusions. Patients receiving deferasirox (Exjade, Novartis, t_{1/2}=8-16 hours) will stop chelation therapy 3 days prior to each of the 6 planned study transfusions. This amount of time off chelation therapy will be sufficient to prevent interference with iron-related assays [99, 100]. In addition, these patients frequently undergo short “chelation holidays” and the adverse effects of stopping chelation therapy for such short periods of time are expected to be minimal.

### Study Site.

The study site in this Example is as described in Example 4.

### RBC Biotinylation.

RBCs will be biotinylated in the Stem Cell Therapy Laboratory, which is about 25 yards away from the Outpatient Apheresis and Transfusion Suite and adjacent to the Blood Bank. The Stem Cell Therapy Laboratory is FACT accredited and uses current Good Tissue Practices (cGTP) to provide allogeneic and autologous hematopoietic stem cell products to patients. Thus, a detailed Standard Operating Procedure will be used to biotinylate RBC aliquots, as described [98]. The procedure is detailed below. All biotinylated RBC products will be tested prior to issue for LPS contamination using a slight modification of the current Standard Operating Procedure employing a limulus lysate assay. Preliminary studies using discarded donor RBCs from the Blood Bank will be performed to validate the adequacy of biotinylation and the sterility of the resulting product.

### Selection of Subjects (Transfusion Recipients).

Sickle cell disease and β-thalassemia patients who receive chronic simple transfusion therapy will be prospectively studied. Table 4 presents an anonymized list of potential patients for this study (based on the specific inclusion and exclusion criteria detailed below). Participation in this study will be unrestricted with respect to gender or ethnicity and limited to age greater than 1 year old. Ethnicity data will be collected for all study subjects. Specific criteria for inclusion and exclusion are:
Inclusion criteria: (i) specific, well-characterized hemoglobinopathy; (ii) chronic simple transfusion therapy (transfusion episodes ≦6 weeks apart in frequency); (iii) chronic iron chelation therapy; (iv) not pregnant by self-report and not planning pregnancy; (v) age >1 year old.
Exclusion criteria: (i) positive RBC antibody screen; (ii) clinically unstable; (iii) treatment for mental illness; (iv) imprisonment; (v) institutionalization.

### Enrollment of Subjects (Transfusion Recipients).

Adult and pediatric patients with hemoglobinopathies who are seen at the Hematology Outpatient Clinic, and who meet the selection criteria, will be identified as potential study subjects (e.g. those in Table 4). These patients will be informed about this study verbally and in writing by an individual other than their treating physician to avoid any conflict-of-interest. Those willing to participate will be enrolled in the study after providing informed consent.

### Selection of Subjects (Transfusion Donors).

Donors from a frequent RBC donor database maintained by the NYBC will be recruited as dedicated directed donors for each subject. Donors must meet NYBC requirements for double RBC donation (e.g. males must weigh more than 130 lbs and be taller than 5′1″; females must weigh more than 150 lbs and be taller than 5′5″). Although New York State allows donors older than 16 years and younger than 76 years, the donor age will be restricted to 21-65 years of age to ensure that the donor has a history of frequent donations and to decrease the donor drop-out due to health or social reasons. All donors will be asked to commit to 4 double RBC donations over a 2-2.5 year period. Donors who do not feel reasonably certain that they will remain in the New York City metropolitan area for the study period will be excluded. In addition, all donors for the β-thalassemia cohort will be ABO and Rh(D) matched and all donors for the sickle cell disease cohort will be ABO, Rh(D,C,c,E,e), and Kell matched as per current standard of practice with these patients at CUMC. Finally, if a dedicated donor drops out of the study after a paired transfusion event has occurred, a new dedicated donor will be recruited for subsequent donations (i.e. this will not automatically lead to exclusion of the transfusion recipient from the study). Specific criteria for inclusion and exclusion are:
Inclusion criteria: (i) 21-65 years of age; (ii) male weight >130 lbs, female weight >150 lbs; (iii) male height >5′1″, female height >5′5″; (iv) hemoglobin >13.3 g/dL; (v) reasonably certain of intention to stay in New York City metropolitan area for study duration; (vi) previously tolerated double RBC donation by apheresis; (vii) frequent donor at NYBC as defined by an average of at least 3 RBC unit donations per year over the past 5 years.
Exclusion criteria: (i) ineligible for donation based on NYBC blood donor questionnaire; (ii) systolic blood pressure <90 or >180 mm Hg, diastolic blood pressure <50 or >100 mm Hg; (iii) heart rate <50 or >100; (iv) temperature >99.5° F. prior to donation; (v) positive by standard infectious disease testing.

### RBC Survival Study In Vivo.

The survival study will be optional (i.e. a patient may choose to opt out of the RBC survival study and still remain in the overall study). In addition, a maximum of one RBC survival study per patient will be performed. For measurement of RBC survival in vivo, a 10 mL aliquot of packed RBCs will be biotinylated as described [98], with minor modifications. In brief, a 10 mL aliquot of packed RBCs will be removed in sterile fashion from the study unit on the day of transfusion. This aliquot will be washed 3 times with 4 volumes of Dulbecco's PBS (Invitrogen), transferred to a 175 mL tube (Nalgene, Rochester, N.Y.), and adjusted to a 7% hematocrit for biotinylation. A stock solution of 2 mg/mL N-hydroxysuccinimide biotin (NHS-biotin) in 10% DMSO will be prepared by dissolving 10 mg of NHS-biotin (Sigma) in 0.5 mL DMSO, followed by addition of 4.5 mL of Dulbecco's PBS. This stock solution will be sterilized by filtration through a 0.2-μm syringe filter (Corning Glassware) made from DMSO-resistant materials. The NHS-biotin stock solution will be added with gentle agitation to the 7% RBC suspension to yield a final NHS-biotin concentration of 1 μg/mL. After 30 minutes incubation at room temperature, the RBCs will be washed twice with at least 3 volumes of Dulbecco's PBS. Two subsequent washes will be performed with injectable isotonic saline, and the RBCs will be re-suspended in 6-10 mL of saline for injection. These RBCs will be injected (5 ml total) by “IV push” prior to transfusion of the RBC unit. Blood samples (1 mL in EDTA) obtained 5 minutes and 1 hour post-injection will be used to calculate the 1-hour RBC survival, and then the study transfusion will begin. Because the overarching belief is that the acute delivery of hemoglobin iron to the monocyte-macrophage system from an older stored RBC transfusion causes a pro-inflammatory response, and because most of the acute RBC clearance occurs within the first hour post-transfusion [33], only the 1-hour RBC survival will be measured.

Blood samples (obtained at 5 minutes and 1 hour post-injection; 1 mL collected into EDTA) will be analyzed by flow cytometry to calculate the percentage of circulating biotinylated RBCs. In brief, 80 μL of a 1% RBC suspension made from these samples will be mixed with 20 μL of a 1:20 dilution (in PBS) of streptavidin-phycoerythrin (Molecular Probes, Invitrogen) and incubated at room temperature for 30 minutes. Following 2 washes with PBS, the RBCs will be re-suspended in PBS and analyzed by flow cytometry. The percentage of phycoerythrin-positive RBCs in the 1-hour post-infusion sample will be compared to the 5-minute sample to estimate 1-hour RBC survival.

### Washed RBCs and Cryopreserved RBCs.

Cryopreservation of one of the two donated double RBC units will be performed using Standard Operating Procedures at the NYBC within 24 hours of collection. Washing older stored RBC units and deglycerolizing cyropreserved RBC units will both be performed within 24 hours of transfusion, also by NYBC Standard Operating Procedures. Deglycerolization and washing of RBC units will each be performed using an automatic cell washing system (COBE 2991, CaridianBCT, Lakewood, Colo.).

### Cytokine Measurements and Other Routine Clinical Laboratory Assays.

All analytes will be measured as described in the Example disclosed above.

### Statistical Considerations.

This is a prospective study of patients with hemoglobinopathies on chronic transfusion therapy, each receiving 3-paired RBC transfusions. Each paired transfusion event will be composed of one control and one experimental transfusion (see FIG. 11 for study outline). The first paired transfusion event will test the belief that transfusion of older stored RBCs induces an acute pro-inflammatory response in chronically transfused patients. This transfusion event will be composed of one control transfusion (i.e. “fresh:” 3-14 days of storage) and one experimental (i.e. “old:” 28-42 days of storage). The primary study outcome will be a paired comparison for each subject of the maximum difference between each pre- and post-transfusion level of 6 cytokines (Table 1), comparing the levels obtained from the “fresh” and “old” RBC transfusions. The other two paired transfusion events will test the subsidiary belief of whether washed older stored RBCs induce a similar pro-inflammatory cytokine response, and whether cryopreservation induces an even greater cytokine response. A subsidiary outcome will be examined by comparing the degree of the cytokine response between sickle cell disease patients, who have a chronic hemolytic state, and β-thalassemia patients, who generally do not.

To provide sample size justification, it is believed that the acute delivery of hemoglobin iron by transfusion of older stored RBCs induces a pro-inflammatory cytokine response; thus, washing an RBC unit should not improve the cytokine response because it does not remove the toxic component (i.e. the aged RBCs themselves), and cryopreservation will make the response greater due to increased clearance of damaged cryopreserved RBCs. The primary study outcome is a paired comparison for each subject of the maximum concentration difference between a post- and pre-transfusion cytokine level (ΔCₘₐₓ) comparing the “fresh” transfusion (stored 3-14 days) and the “old” transfusion (stored 28-42 days). Therefore, the sample size is estimated only with respect to this primary outcome. Subsidiary analyses will be made with respect to other study outcomes, but these comparisons were not entered into the sample size calculation.

Based on the pre-clinical data in mice, the infusion of the equivalent of one unit of fresh and older stored RBCs led to a ΔCₘₐₓ of plasma MCP-1 (a robust pro-inflammatory cytokine) of 386.4 pg/mL with a standard deviation of 138.3 pg/mL, when comparing the value obtained from fresh and older stored RBC unit transfusions. Statistically, the primary outcome measure is similar to Example 4. Thus, using a paired two-sample t-test with a two-sided significance level of 0.05, an expected standard deviation of 138.3 pg/mL, and a power of 0.80, approximately 8.9 subjects will be needed to detect a difference of 150 pg/mL. This is about 40% of the difference seen in mice. Thus, to complete the study, 9 patients from each group (i.e. about 50% of the sickle cell disease patients and 100% of the β-thalassemia patients listed in Table 4) will be needed. Although the required number of individuals may be successfully recruited and retained from the patients listed in Table 4, it is possible that additional patients will need to be identified. To this end, the Pediatric Hematology Division at CUMC is a dynamic and expanding clinical service. In addition, other patients from the New York metropolitan area with sickle disease and β-thalassemia are referred to CUMC and cared for by the adult hematologists. Therefore, should additional patients be needed, they will be readily available.

Similar considerations apply for the subsidiary beliefs, which test the effects of washed RBCs and cryopreserved RBCs. Using a paired two-sample t-test with a two-sided significance level of 0.05 and a power of 0.80, a difference in the ΔCₘₐₓ of plasma MCP-1 levels of at least 150 pg/mL may be detected when comparing the levels obtained from the untreated “old” RBC transfusion with either the washed “old” RBC or cryopreserved RBC transfusion.

This sample size also provides adequate power for the subsidiary belief testing whether chronic hemolysis mitigates against the pro-inflammatory response (i.e. comparing cytokine levels in sickle cell disease and β-thalassemia patients). Using an unpaired two-sample t-test with a two-sided significance level of 0.05 and a power of 0.80, a difference in the ΔCₘₐₓ of plasma MCP-1 levels of at least 195 pg/mL between sickle cell disease and β-thalassemia patients may be detected. This represents 50% of the cytokine difference seen in mice.

It is expected that transfusion of older stored RBCs induces an acute pro-inflammatory response in chronically transfused patients with sickle cell disease or β-thalassemia.

### Example 8

### Treating Sickle Cell Disease and β-Thalassemia Patients with Iron Chelators Will Prevent the Acute Pro-Inflammatory Response Induced by Transfusion of Older Stored RBCs.

The primary study belief is that iron chelation will ameliorate pro-inflammatory cytokine responses in patients with sickle cell disease and β-thalassemia.

### Overview.

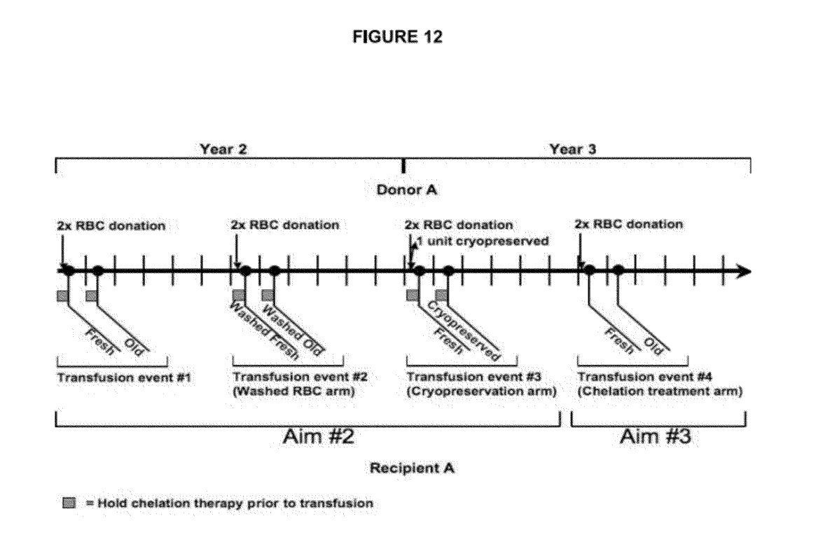

This experiment will test a potential therapeutic intervention. Thus, this experiment simply represents a continuation of the two-year study disclosed in Example 5 to include a paired transfusion event of “fresh” (i.e. 3-14 days of storage) and “old” (i.e. 28-42 days of storage) RBC units while the patients remain on iron chelation therapy. In the 3 paired transfusion events disclosed in Example 5, the patients will temporarily stop chelation therapy prior to transfusion; in this experimental setup, the pro-inflammatory cytokine response while on chelation will be measured, and the results will be compared to those obtained in the first paired transfusion event described in Example 5. The same dedicated donors will be used and the same analytes will be measured. Thus, the full study is summarized in FIG. 12; although documented in this figure as occurring in the second and third years of this four-year project, it is expected that these studies will continue into the fourth year for some of the patients. For example, if 18 patients are enrolled, then this will require a total 144 transfusions (i.e. 18×8); the goal will be to perform about 1.1 transfusion per week, on average.

### Statistical Considerations.

This is a prospective study of hemoglobinopathy patients receiving two RBC transfusions while on chelation therapy and two RBC transfusions while off chelation therapy, one control (i.e. “fresh:” 3-14 days of storage), and one experimental (i.e. “old:” 28-42 days of storage). The primary study outcome will be a paired comparison for each subject of the maximum difference between each pre- and post-transfusion level of 6 cytokines (Table 1), between the “fresh” and “old” RBC transfusions, comparing this difference on chelation therapy with the value obtained off chelation therapy.

To provide sample size justification, it is hypothesized that the acute delivery of hemoglobin iron by transfusion of older stored RBC induces a pro-inflammatory cytokine response; therefore, iron chelation should mitigate against this response. The primary study outcome is a paired comparison for each subject of the maximum concentration difference between a post- and pre-transfusion cytokine level (ΔCₘₐₓ) and between the “fresh” transfusion event (i.e. stored 3-14 days) and the “old” transfusion event (i.e. stored 28-42 days), and comparing this value while the patient is on chelation therapy to the value obtained off chelation therapy. Based on the pre-clinical data in mice, iron chelation reduces the pro-inflammatory cytokine response by more than 50%. In addition, the transfusion of the equivalent of one unit of fresh and older stored RBCs led to a ΔCₘₐₓ of plasma MCP-1 (a robust pro-inflammatory cytokine) of 386.4 pg/mL, with a standard deviation of 138.3 pg/mL, when comparing the fresh and older stored RBC unit transfusions. Statistically, the primary outcome measure in this Example is similar to that of Examples 4 and 5. Thus, if a paired two-sample t-test with a two-sided significance level of 0.05, an expected standard deviation of 138.3 pg/mL, and a power of 0.80, is used, then about 8.9 subjects are required to detect a difference of 150 pg/mL. This is about 40% of the difference seen in mice and adequately powers this study to detect a 50% difference in cytokine levels with chelation therapy. Nine sickle cell disease patients and 9 β-thalassemia patients will be needed to adequately power the study disclosed above. Using this conservative sample size estimate and a power of 0.80, a 40% difference in cytokine levels may be detected, which is still below the 50% effect seen in mice.

### Results.

It is expected that treating sickle cell disease and β-thalassemia patients with iron chelators will prevent the acute pro-inflammatory response induced by transfusion of older stored RBCs.

The present invention is believed to be the first to demonstrate transfusion of older stored leukoreduced RBCs induces a pro-inflammatory response. Accordingly, acute delivery, by virtually any mechanism, of substantial amounts of hemoglobin iron to the monocyte-macrophage system induces oxidative stress, thereby eliciting secretion of pro-inflammatory cytokines.

Based on prior observations of human patients and the pre-clinical results in mice disclosed herein, it is believed that the clinical signs and symptoms resulting from transfusions of older stored RBCs will vary depending on the recipient's underlying disease state. For example, although transfusions of healthy mice with older stored RBCs did not produce obvious symptoms, dramatic responses were seen after RBC transfusions in mice that were infused with small amounts of LPS, leading to prolongation and exacerbation of cytokine storm (FIG. 8). Therefore, studies will be conducted based on two disease settings that require chronic RBC transfusions: sickle cell disease and β-thalassemia. Using a design in which patients serve as their own controls and where donor exposure is minimized, whether an underlying hemolytic state (i.e. sickle cell disease vs. β-thalassemia) affects the pro-inflammatory response to transfusions using older stored RBCs will be examined. Whether other standard RBC products induce a pro-inflammatory response following transfusion will also be determined; these include washed RBCs, which are used extensively in some centers, and cryopreserved RBCs, which may be required for highly alloimmunized patients, and which sustain some damage in vitro.

Finally, the routine use of iron chelation therapy in these patients to prevent iron overload from chronic transfusions will allow for the determination of whether iron chelation inhibits the acute pro-inflammatory response that occurs immediately after the transfusion of older stored RBCs.

Positive findings resulting from this study (i.e. that older stored RBCs indeed do elicit an acute pro-inflammatory response in healthy volunteers or patients) will immediately impact current transfusion medicine practice and will begin to provide the scientific basis for an evidence-based approach regarding identifying the appropriate RBC storage interval(s) prior to transfusion. The results may differ for different patient groups (e.g. the chronic hemolytic state in sickle cell disease may induce refractoriness to the acute adverse effects of older stored RBC transfusions). However, negative findings (i.e. that transfusions of older stored RBCs do not elicit an acute pro-inflammatory response in humans) will also have broad implications for the current practice of transfusion medicine; thus, this would provide reassurance that the current RBC storage standards are appropriate.

Interestingly, these studies may also elucidate one currently inexplicable observation; that is, febrile transfusion reactions are still induced by transfusion of leukoreduced RBCs (although less commonly than before leukoreduction). The cause of these reactions is either left unexplained or ascribed to less than optimal leukoreduction of the inciting RBC unit. Some of these cases may result from over exuberant secretion of pro-inflammatory cytokines by the monocyte-macrophage system after acute delivery of hemoglobin iron by the clearance of stored RBCs. It is predicted that such “reactions” will be more prevalent when older RBC units are used for transfusion. It is also possible that host-specific factors (e.g. genetic polymorphisms in cytokine genes [104]) may predispose certain transfusion recipients to develop such reactions. Indeed, this may represent a new type of transfusion reaction secondary to non-immunologically-mediated extravascular hemolysis of older stored RBCs.

### INCORPORATION BY REFERENCE

All publications and patents referred to herein or listed below are hereby incorporated by reference in their entirety as if each individual publication or patent was specifically and individually indicated to be incorporated by reference. In case of conflict, the present application, including any definitions herein, will control.

### DOCUMENTS CITED

[1] Whitaker B, Sullivan M. The 2005 Nationwide Blood Collection and Utilization Survey Report. Bethesda, Md.: AABB; 2006.
[2] Duke E M. Report to Congress—The Critical Care Workforce: A study of the Supply and Demand for Critical Care Physicians. In: Services UDoHH, editor. The Critical Care Workforce.
[3] Corwin H L, Gettinger A, Pearl R G, Fink M P, Levy M M, Abraham E, MacIntyre NR, Shabot M M, Duh M S, Shapiro M J. The CRIT Study: Anemia and blood transfusion in the critically ill—current clinical practice in the United States. Crit Care Med 2004; 32: 39-52.
[4] Koch C G, Li L, Van Wagoner D R, Duncan A I, Gillinov A M, Blackstone E H. Red cell transfusion is associated with an increased risk for postoperative atrial fibrillation. Ann Thorac Surg 2006; 82: 1747-1756.
[5] Engoren M C, Habib R H, Zacharias A, Schwann T A, Riordan C J, Durham S J. Effect of blood transfusion on long-term survival after cardiac operation. Ann Thorac Surg 2002; 74: 1180-1186.
[6] Zacharias A, Habib R H. Factors predisposing to median sternotomy complications. Deep vs superficial infection. Chest 1996; 110: 1173-1178.
[7] Leal-Noval S R, Rincon-Ferrari M D, Garcia-Curiel A, Herruzo-Aviles A, Camacho-Larana P, Garnacho-Montero J, Amaya-Villar R. Transfusion of blood components and postoperative infection in patients undergoing cardiac surgery. Chest 2001; 119: 1461-1468.
[8] Taylor R W, Manganaro L, O'Brien J, Trottier S J, Parkar N, Veremakis C. Impact of allogenic packed red blood cell transfusion on nosocomial infection rates in the critically ill patient. Crit. Care Med 2002; 30: 2249-2254.
[9] Malone D L, Dunne J, Tracy J K, Putnam A T, Scalea T M, Napolitano L M. Blood transfusion, independent of shock severity, is associated with worse outcome in trauma. J Trauma 2003; 54: 898-905; discussion 905-897.
[10] Hebert P C, Wells G, Blajchman M A, Marshall J, Martin C, Pagliarello G, Tweeddale M, Schweitzer I, Yetisir E. A multicenter, randomized, controlled clinical trial of transfusion requirements in critical care. Transfusion Requirements in Critical Care Investigators, Canadian Critical Care Trials Group. N Engl J Med 1999; 340: 409-417.
[11] Purdy F R, Tweeddale M G, Merrick P M. Association of mortality with age of blood transfused in septic ICU patients. Can J Anaesth 1997; 44: 1256-1261.
[12] Koch C G, Li L, Sessler D I, Figueroa P, Hoeltge G A, Mihaljevic T, Blackstone E H. Duration of red-cell storage and complications after cardiac surgery. N Engl J Med 2008; 358: 1229-1239.
[13] Basran S, Frumento R J, Cohen A, Lee S, Du Y, Nishanian E, Kaplan H S, Stafford-Smith M, Bennett-Guerrero E. The association between duration of storage of transfused red blood cells and morbidity and mortality after preoperative cardiac surgery. Anesth Analg 2006; 103: 15-20, table of contents.
[14] Offner P J, Moore E E, Biffl W L, Johnson J L, Silliman C C. Increased rate of infection associated with transfusion of old blood after severe injury. Arch Surg 2002; 137: 711-716; discussion 716-717.
[15] Zallen G, Offner P J, Moore E E, Blackwell J, Ciesla D J, Gabriel J, Denny C, Silliman C C. Age of transfused blood is an independent risk factor for postinjury multiple organ failure. Am J Surg 1999; 178: 570-572.
[16] Dzik W. Fresh blood for everyone? Balancing availability and quality of stored RBC. Transfus Med 2008; 18: 260-265.
[17] Tinmouth A, Fergusson D, Yee I C, Hebert P C. Clinical consequences of red cell storage in the critically ill. Transfusion 2006; 46: 2014-2027.
[18] Bordin J O, Heddle N M, Blajchman M A. Biologic effects of leukocytes present in transfused cellular blood products. Blood 1994; 84: 1703-1721.
[19] Weinberg J A, McGwin G, Jr., Griffin R L, Huynh V Q, Chemy S A, 3rd, Marques M B, Reiff D A, Kerby J D, Rue L W, 3rd. Age of transfused blood: an independent predictor of mortality despite universal leukoreduction. J Trauma 2008; 65: 279-282; discussion 282-274.
[20] Pietersz R N, Reesink H W, de Korte D, Dekker W J, van den Ende A, Loos J A. Storage of leukocyte-poor red cell concentrates: filtration in a closed system using a sterile connection device. Vox Sang 1989; 57: 29-36.
[21] Valeri C R, Hirsch N M. Restoration in vivo of erythrocyte adenosine triphosphate, 2,3-diphosphoglycerate, potassium ion, and sodium ion concentrations following the transfusion of acid-citrate-dextrose-stored human red blood cells. J Lab Clin Med 1969; 73: 722-733.
[22] Greenwalt T J, Bryan D J, Dumaswala U J. Erythrocyte membrane vesiculation and changes in membrane composition during storage in citrate-phosphate-dextrose-adenine-1. Vox Sang 1984; 47: 261-270.
[23] Wagner G M, Chiu D T, Qju J H, Heath R H, Lubin B H. Spectrin oxidation correlates with membrane vesiculation in stored RBC. Blood 1987; 69: 1777-1781.
[24] Knight J A, Voorhees R P, Martin L, Anstall H. Lipid peroxidation in stored red cells. Transfusion 1992; 32: 354-357.
[25] Reynolds J D, Ahearn G S, Angelo M, Zhang J, Cobb F, Stamler J S. S-nitrosohemoglobin deficiency: a mechanism for loss of physiological activity in banked blood. Proc Natl Acad Sci USA 2007; 104: 17058-17062.
[26] Hovav T, Yedgar S, Manny N, Barshtein G. Alteration of red cell aggregability and shape during blood storage. Transfusion 1999; 39: 277-281.
[27] Holovati J L, Wong K A, Webster J M, Acker J P. The effects of cryopreservation on red blood cell microvesiculation, phosphatidylserine externalization, and CD47 expression. Transfusion 2008; 48: 1658-1668.
[28] Verhoeven A J, Hilarius P M, Dekkers D W, Lagerberg J W, de Korte D. Prolonged storage of red blood cells affects aminophospholipid translocase activity. Vox Sang 2006; 91: 244-251.
[29] Card R T, Mohandas N, Perkins H A, Shohet S B. Deformability of stored red blood cells. Relationship to degree of packing. Transfusion 1982; 22: 96-101.
[30] Hogman C F, Meryman H T. Storage parameters affecting red blood cell survival and function after transfusion. Transfus Med Rev 1999; 13: 275-296.
[31] Marwah S S, Blann A, Harrison P, Lumley M A, Wright J, McDowell J, Phillips J D, Rea C, Bareford D. Increased non-transferrin bound iron in plasma-depleted SAG-M red blood cell units. Vox Sang 2002; 82: 122-126.
[32] Dumont L J, AuBuchon J P. Evaluation of proposed FDA criteria for the evaluation of radiolabeled red cell recovery trials. Transfusion 2008; 48: 1053-1060.
[33] Luten M, Roerdinkholder-Stoelwinder B, Schaap N P, de Grip W J, Bos H J, Bosman G J. Survival of red blood cells after transfusion: a comparison between red cells concentrates of different storage periods. Transfusion 2008; 48: 1478-1485.
[34] Zeiler T, Muller J T, Kretschmer V. Flow-cytometric determination of survival time and 24-hour recovery of transfused red blood cells. Transfus Med Hemother 2003; 30: 14-19.
[35] Shi X, Dong Z, Huang C, Ma W, Liu K, Ye J, Chen F, Leonard S S, Ding M, Castranova V, Vallyathan V. The role of hydroxyl radical as a messenger in the activation of nuclear transcription factor NF-kappaB. Mol Cell Biochem 1999; 194: 63-70.
[36] Kaul N, Forman H J. Activation of NF kappa B by the respiratory burst of macrophages. Free Radic Biol Med 1996; 21: 401-405.
[37] Halliwell B. Superoxide-dependent formation of hydroxyl radicals in the presence of iron chelates: is it a mechanism for hydroxyl radical production in biochemical systems? FEBS Lett 1978; 92: 321-326.
[38] Xiong S, She H, Takeuchi H, Han B, Engelhardt J F, Barton C H, Zandi E, Giulivi C, Tsukamoto H. Signaling role of intracellular iron in NF-kappaB activation. J Biol Chem 2003; 278: 17646-17654.
[39] Jaramillo M, Olivier M. Hydrogen peroxide induces murine macrophage chemokine gene transcription via extracellular signal-regulated kinase- and cyclic adenosine 5′-monophosphate (cAMP)-dependent pathways: involvement of NF-kappa B, activator protein 1, and cAMP response element binding protein. J Immunol 2002; 169: 7026-7038.
[40] Wang L, Johnson E E, Shi H N, Walker W A, Wessling-Resnick M, Cherayil B J. Attenuated inflammatory responses in hemochromatosis reveal a role for iron in the regulation of macrophage cytokine translation. J Immunol 2008; 181: 2723-2731.
[41] Tsukamoto H, Lin M, Ohata M, Giulivi C, French S W, Brittenham G. Iron primes hepatic macrophages for NF-kappaB activation in alcoholic liver injury. Am J Physiol 1999; 277: G1240-1250.
[42] Gordeuk V R, Ballou S, Lozanski G, Brittenham G M. Decreased concentrations of tumor necrosis factor-alpha in supernatants of monocytes from homozygotes for hereditary hemochromatosis. Blood 1992; 79: 1855-1860.
[43] Fransen E, Maessen J, Dentener M, Senden N, Buurman W. Impact of blood transfusions on inflammatory mediator release in patients undergoing cardiac surgery. Chest 1999; 116: 1233-1239.
[44] Avall A, Hyllner M, Bengtson J P, Carlsson L, Bengtsson A. Postoperative inflammatory response after autologous and allogeneic blood transfusion. Anesthesiology 1997; 87: 511-516.
[45] Bengtsson A, Avall A, Hyllner M, Bengtson J P. Formation of complement split products and proinflammatory cytokines by reinfusion of shed autologous blood. Toxicol Lett 1998; 100-101: 129-133.
[46] Mendez C, Garcia I, Maier R V . Oxidants augment endotoxin-induced activation of alveolar macrophages. Shock 1996; 6: 157-163.
[47] Erwig L P, Kluth D C, Walsh G M, Rees A J. Initial cytokine exposure determines function of macrophages and renders them unresponsive to other cytokines. J Immunol 1998; 161: 1983-1988.
[48] Gordon S. Alternative activation of macrophages. Nat Rev Immunol 2003; 3: 23-35.
[49] Mosser D M. The many faces of macrophage activation. J Leukoc Biol 2003; 73: 209-212.
[50] Tsuda Y, Takahashi H, Kobayashi M, Hanafusa T, Herndon D N, Suzuki F. CCL2, a product of mice early after systemic inflammatory response syndrome (SIRS), induces alternatively activated macrophages capable of impairing antibacterial resistance of SIRS mice. J Leukoc Biol 2004; 76: 368-373.
[51] Takahashi H, Tsuda Y, Takeuchi D, Kobayashi M, Herndon D N, Suzuki F. Influence of systemic inflammatory response syndrome on host resistance against bacterial infections. Crit. Care Med 2004; 32: 1879-1885.
[52] Belkaid Y. Regulatory T cells and infection: a dangerous necessity. Nat Rev Immunol 2007; 7: 875-888.
[53] Young P P, Uzieblo A, Trulock E, Lublin D M, Goodnough L T. Autoantibody formation after alloimmunization: are blood transfusions a risk factor for autoimmune hemolytic anemia? Transfusion 2004; 44: 67-72.
[54] Tiemessen M M, Jagger A L, Evans H G, van Herwijnen M J, John S, Taams L S. CD4+CD25+Foxp3+ regulatory T cells induce alternative activation of human monocytes/macrophages. Proc Natl Acad Sci USA 2007; 104: 19446-19451.
[55] Hider R C. Nature of nontransferrin-bound iron. Eur J Clin Invest 2002; 32 Suppl 1:50-54.
[56] Hershko C. Mechanism of iron toxicity. Food Nutr Bull 2007; 28: S500-509.
[57] van Tits L J, Jacobs E M, Swinkels D W, Lemmers H L, van der Vleuten G M, de Graaf J, Stalenhoef A F. Non-transferrin-bound iron is associated with plasma level of soluble intercellular adhesion molecule-1 but not with in vivo low-density lipoprotein oxidation. Atherosclerosis 2007; 194: 272-278.
[58] Esposito B P, Breuer W, Sirankapracha P, Pootrakul P, Hershko C, Cabantchik Z I. Labile plasman iron in iron overload: redox activity and susceptibility to chelation. Blood 2003; 102: 2670-2677.
[59] Dani C, Martelli E, Bertini G, Pezzati M, Rossetti M, Buonocore G, Paffetti P, Rubaltelli F F. Effect of blood transfusions on oxidative stress in preterm infants. Arch Dis Child Fetal Neonatal Ed 2004; 89: F408-411.
[60] Ozment C P, Turi J L. Iron overload following red blood cell transfusion and its impact on disease severity. Biochim Biophys Acta 2008.
[61] Turhan A, Weiss L A, Mohandas N, Coller B S, Frenette P S. Primary role for adherent leukocytes in sickle cell vascular occlusion: a new paradigm. Proc Natl Acad Sci USA 2002; 99: 3047-3051.
[62] Adams R J, McKie V C, Hsu L, Files B, Vichinsky E, Pegelow C, Abboud M, Gallagher D, Kutlar A, Nichols F T, Bonds D R, Brambilla D. Prevention of a first stroke by transfusions in children with sickle cell anemia and abnormal results on transcranial Doppler ultrasonography. N Engl J Med 1998; 339: 5-11.
[63] Afenyi-Annan A, Willis M S, Konrad T R, Lottenberg R. Blood bank management of sickle cell patients at comprehensive sickle cell centers. Transfusion 2007; 47: 2089-2097.
[64] Wayne A S, Kevy S V, Nathan D G. Transfusion management of sickle cell disease. Blood 1993; 81: 1109-1123.
[65] Schmalzer E A, Lee J O, Brown A K, Usami S, Chien S. Viscosity of mixtures of sickle and normal red cells at varying hematocrit levels. Implications for transfusion. Transfusion 1987; 27: 228-233.
[66] Graido-Gonzalez E, Doherty J C, Bergreen E W, Organ G, Telfer M, McMillen M A. Plasma endothelin-1, cytokine, and prostaglandin E2 levels in sickle cell disease and acute vaso-occlusive sickle crisis. Blood 1998; 92: 2551-2555.
[67] Jison M L, Munson P J, Barb J J, Suffredini A F, Talwar S, Logun C, Raghavachari N, Beigel J H, Shelhamer J H, Danner R L, Gladwin M T. Blood mononuclear cell gene expression profiles characterize the oxidant, hemolytic, and inflammatory stress of sickle cell disease. Blood 2004; 104: 270-280.
[68] Rodrigues L, Costa F F, Saad S T, Grotto H Z. High levels of neopterin and interleukin-3 in sickle cell disease patients. J Clin Lab Anal 2006; 20: 75-79.
[69] Brittain J E, Parise L V. Cytokines and plasma factors in sickle cell disease. Curr Opin Hematol 2007; 14: 438-443.
[70] Hibbert J M, Hsu L L, Bhathena S J, Irune I, Sarfo B, Creary M S, Gee B E, Mohamed A I, Buchanan I D, Al-Mahmoud A, Stiles J K. Proinflammatory cytokines and the hypermetabolism of children with sickle cell disease. Exp Biol Med (Maywood) 2005; 230: 68-74.
[71] Walter P B, Fung E B, Killilea D W, Jiang Q, Nudes M, Madden J, Porter J, Evans P, Vichinsky E, Harmatz P. Oxidative stress and inflammation in iron-overloaded patients with beta-thalassaemia or sickle cell disease. Br J Haematol 2006; 135: 254-263.
[72] Fan H, Cook J A. Molecular mechanisms of endotoxin tolerance. J Endotoxin Res 2004; 10: 71-84.
[73] Zager R A, Johnson A C, Lund S. ‘Endotoxin tolerance’: TNF-alpha hyper-reactivity and tubular cytoresistance in a renal cholesterol loading state. Kidney Int 2007; 71: 496-503.
[74] Rund D, Rachmilewitz E. Beta-thalassemia. N Engl J Med 2005; 353: 1135-1146.
[75] Kyriakou D S, Alexandrakis M G, Kyriakou E S, Liapi D, Kourelis T V, Passam F, Papadakis A. Activated peripheral blood and endothelial cells in thalassemia patients. Ann Hematol 2001; 80: 577-583.
[76] Aggeli C, Antoniades C, Cosma C, Chrysohoou C, Tousoulis D, Ladis V, Karageorga M, Pitsavos C, Stefanadis C. Endothelial dysfunction and inflammatory process in transfusion-dependent patients with beta-thalassemia major. Int J Cardiol 2005; 105: 80-84.
[77] Grosse R, Lund U, Caruso V, Fischer R, Janka G E, Magnano C, Engelhardt R, Durken M, Nielsen P. Non-transferrin-bound iron during blood transfusion cycles in beta-thalassemia major. Ann N Y Acad Sci 2005; 1054: 429-432.
[78] Porter J B, Rafique R, Srichairatanakool S, Davis B A, Shah F T, Hair T, Evans P. Recent insights into interactions of deferoxamine with cellular and plasman iron pools: Implications for clinical use. Ann N Y Acad Sci 2005; 1054: 155-168.
[79] Cighetti G, Duca L, Bortone L, Sala S, Nava I, Fiorelli G, Cappellini M D. Oxidative status and malondialdehyde in beta-thalassaemia patients. Eur J Clin Invest 2002; 32 Suppl 1: 55-60.
[80] Grabmer C, Holmberg J, Popovsky M, Amann E, Schonitzer D, Falaize S, Hanske H, Pages E, Nussbaumer W. Up to 21-day banked red blood cells collected by apheresis and stored for 14 days after automated wash at different times of storage. Vox Sang 2006; 90: 40-44.
[81] Vichinsky E P, Luban N L, Wright E, Olivieri N, Driscoll C, Pegelow C H, Adams R J. Prospective RBC phenotype matching in a stroke-prevention trial in sickle cell anemia: a multicenter transfusion trial. Transfusion 2001; 41: 1086-1092.
[82] Valeri C R, Ragno G, Pivacek L, O'Neill E M. In vivo survival of apheresis RBC, frozen with 40-percent (wt/vol) glycerol, deglycerolized in the ACP 215, and stored at 4 degrees C. in AS-3 for up to 21 days. Transfusion 2001; 41: 928-932.
[83] Valeri C R, Ragno G, Pivacek L E, Srey R, Hess J R, Lippert L E, Mettille F, Fahie R, O'Neill E M, Szymanski 10. A multicenter study of in vitro and in vivo values in human RBC frozen with 40-percent (wt/vol) glycerol and stored after deglycerolization for 15 days at 4 degrees C. in AS-3: assessment of RBC processing in the ACP 215. Transfusion 2001; 41: 933-939.
[84] Grose H L, Byrne K M, Salata J M, Rentas F J, Stroncek D F. In vitro variables of red blood cell components collected by apheresis and frozen 6 and 14 days after collection. Transfusion 2006; 46: 1178-1183.
[85] Gilson C R, Kraus T, Hod E A, Hendrickson J, Spitalnik S L, Hillyer C D, Shaz B, Zimring J C. Mouse RBC Stored in CPDA-1 Have Similar Characteristics to the Established Properties of Stored Human RBC. Transfusion 2008; Submitted.
[86] Pasini E M, Kirkegaard M, Salerno D, Mortensen P, Mann M, Thomas A W. Deep coverage mouse red blood cell proteome: a first comparison with the human red blood cell. Mol Cell Proteomics 2008; 7: 1317-1330.
[87] Schirmer D A, Song S C, Baliff J P, Harbers S O, Clynes R A, Krop-Watorek A, Halverson G R, Czerwinski M, Spitalnik S L. Mouse models of IgG- and IgM-mediated hemolysis. Blood 2007; 109: 3099-3107.
[88] Overmoyer B A, McLaren C E, Brittenham G M. Uniformity of liver density and nonheme (storage) iron distribution. Arch Pathol Lab Med 1987; 111: 549-554.
[89] Dodge J T, Mitchell C, Hanahan D J. The preparation and chemical characteristics of hemoglobin-free ghosts of human erythrocytes. Arch Biochem Biophys 1963; 100: 119-130.
[90] Lee M T, Piomelli S, Granger S, Miller S T, Harkness S, Brambilla D J, Adams R J. Stroke Prevention Trial in Sickle Cell Anemia (STOP): extended follow-up and final results. Blood 2006; 108: 847-852.
[91] Sheth S, Tang H, Jensen J H, Altmann K, Prakash A, Printz B F, Hordof A J, Tosti C L, Azabagic A, Swaminathan S, Brown T R, Olivieri N F, Brittenham G M. Methods for noninvasive measurement of tissue iron in Cooley's anemia. Ann N Y Acad Sci 2005; 1054: 358-372.
[92] Nemeth E, Valore E V, Territo M, Schiller G, Lichtenstein A, Ganz T. Hepcidin, a putative mediator of anemia of inflammation, is a type II acute-phase protein. Blood 2003; 101: 2461-2463.
[93] Zimmermann M B, Fucharoen S, Winichagoon P, Sirankapracha P, Zeder C, Gowachirapant S, Judprasong K, Tanno T, Miller J L, Hurrell R F. Iron metabolism in heterozygotes for hemoglobin E (HbE), alpha-thalassemia 1, or beta-thalassemia and in compound heterozygotes for HbE/beta-thalassemia. Am J Clin Nutr 2008; 88: 1026-1031.
[94] Gosriwatana I, Loreal O, Lu S, Brissot P, Porter J, Hider R C. Quantification of non-transferrin-bound iron in the presence of unsaturated transferrin. Anal Biochem 1999; 273: 212-220.
[95] Evans R W, Rafique R, Zarea A, Rapisarda C, Cammack R, Evans P J, Porter J B, Hider R C. Nature of non-transferrin-bound iron: studies on iron citrate complexes and thalassemic sera. J Biol Inorg Chem 2008; 13: 57-74.
[96] Ganz T, Olbina G, Girelli D, Nemeth E, Westerman M. Immunoassay for human serum hepcidin. Blood 2008; 112: 4292-4297.
[97] Winterbourn C C. Reactions of superoxide with hemoglobin. In: Greenwald R A, editor. Handbook of Methods for Oxygen Radical Research. Boca Raton: CRC; 1985. p. 137-141.
[98] Franco R S, Lohmann J, Silberstein E B, Mayfield-Pratt G, Palascak M, Nemeth T A, Joiner C H, Weiner M, Rucknagel D L. Time-dependent changes in the density and hemoglobin F content of biotin-labeled sickle cells. J Clin Invest 1998; 101: 2730-2740.
[99] Porter J B, Abeysinghe R D, Marshall L, Hider R C, Singh S. Kinetics of removal and reappearance of non-transferrin-bound plasman iron with deferoxamine therapy. Blood 1996; 88: 705-713.
[100] Wang W C, Ahmed N, Hanna M. Non-transferrin-bound iron in long-term transfusion in children with congenital anemias. J Pediatr 1986; 108: 552-557.
[101] Holt J T, Spitalnik S L, McMican A E, Wilson G, Blumberg N. A technetium-99m red cell survival technique for in vivo compatibility testing. Transfusion 1983; 23: 148-151.
[102] Hod E A, Cadwell C M, Liepkalns J S, Zimring J C, Sokol S A, Schirmer D A, Jhang J, Spitalnik S L. Cytokine storm in a mouse model of IgG-mediated hemolytic transfusion reactions. Blood 2008; 112: 891-894.
[103] Davenport R D, Burdick M, Moore S A, Kunkel S L. Cytokine production in IgG-mediated red cell incompatibility. Transfusion 1993; 33: 19-24.
[104] Ness R B, Haggerty C L, Harger G, Ferrell R. Differential distribution of allelic variants in cytokine genes among African Americans and White Americans. Am J Epidemiol 2004; 160: 1033-1038.
[105] Wiltbank T B, Giordano G F. The safety profile of automated collections: an analysis of more than 1 million collections. Transfusion 2007; 47: 1002-1005.
[106] Moore, G. L., Ledford, M. E. & Merydith, A. A micromodification of the Drabkin hemoglobin assay for measuring plasma hemoglobin in the range of 5 to 2000 mg/dl. Biochem. Med. 26, 167-173 (1981).
[107] Zhang, D., Okada, S., Kawabata, T. & Yasuda, T. An improved simple colorimetric method for quantitation of non-transferrin-bound iron in serum. Biochem. Mol. Biol. Int. 35, 635-641 (1995).
[108] Zhang, N., Ahsan, M. H., Purchio, A. F. & West, D. B. Serum amyloid A-luciferase transgenic mice: response to sepsis, acute arthritis, and contact hypersensitivity and the effects of proteasome inhibition. J. Immunol. 174, 8125-8134 (2005).
[109] Gilson, C. R., et al. A novel mouse model of red blood cell storage and posttransfusion in vivo survival. Transfusion. In Press (2009).
[110] Varaprased et al. “Hydroxamic acid polymers. Effect of structure on the chelation of iron in water” Journal of Polymer Science 22: 2131-3143 (1985)
[111] Lemma et. al. “Iron (III) chelation and antioxidant properties of myo-inositol phosphorylated microspheres” Journal of Pharmacy and Pharmacology 59: 597-601 (2007).
[112] Poreddy et. al. “Hydroxamate based iron chelators: combinatorial syntheses of desferroxamine B analogues and evaluation of binding affinities” J. Comb. Chem. 6: 239-254 (2004).
[113] Park et. al. “Novel iron complexed and chelators based on cis, cis-1,3,5-triaminocyclohexane: iron-mediated ligand oxidation and biochemical properties” J. Biol. Inorg. Chem. 3: 449-457 (1998)
[114] Chong et. al. “Synthesis and potent anti-tumor activities of novel 1,3,5-cis,cis-triaminocyclohexane N-pyridyl derivatives.” J. Med. Chem. 47: 5230-5234 (2004).
[115] Kalinowski et. al. “The evolution of iron chelators for the treatment of iron overload disease and cancer” Pharma. Rev. 57: 547-583 (2005).
[116] Schelonka R L, Chaff M K, Yoder B A, Hensley D, Brockett R M, Ascher D P. “Volume of blood required to detect common neonatal pathogens.” J Pediatr. 129(2):275-278 (1996).
[117] Neupane G P, Kim D M. “Comparison of the effects of deferasirox, deferiprone, and deferoxamine on the growth and virulence of Vibrio vulnificus.” Transfusion. 49(8):1762-1769 (2009).
[118] Rao G H, Cox A C, Gerrard J M, White J G. “Effects of 2,2′-dipyridyl and related compounds on platelet prostaglandin synthesis and platelet function.” Biochim Biophys Acta.628(4):468-479 (1980).
[119] Vulcano M, Meiss R P, Isturiz M A. “Deferoxamine reduces tissue injury and lethality in LPS-treated mice.” Int J Immunopharmacol. 22(8):635-644 (2000).
[120] Kaye D, Hook E W. “The influence of hemolysis or blood loss on susceptibility to infection.” J. Immunol. 91:65-75 (1963).
[121] Kaye D, Gill F A, Hook E W. “Factors influencing host resistance to Salmonella infections: the effects of hemolysis and erythrophagocytosis.” Am J Med. Sci. 254(2):205-215 (1967).
[122] Bullen J, Rogers H, Spalding P, Ward C. “Iron and infection: the heart of the matter.” FEMS Immunol Med. Microbiol. 43(3):325-330 (2005).
[123] Mangalmurti N S, Xiong Z, Hulver M, et al. “Loss of red cell chemokine scavenging promotes transfusion-related lung inflammation.” Blood. 113(5):1158-1166 (2009).
[124] Hider R C. Nature of nontransferrin-bound iron. Eur J Clin Invest. 32(suppl 1):50-54 (2002).
[125] Hershko C. Mechanism of iron toxicity. Food Nutr Bull. 28(4 Suppl):S500-S509 (2007).
[126] Remick D G, Villarete L. Regulation of cytokine gene expression by reactive oxygen and reactive nitrogen intermediates. J Leukoc Biol. 59(4): 471-475 (1996).
[127] Schreck R, Albermann K, Baeuerle P A. Nuclear factor kappaB: an oxidative stress-responsive transcription factor of eukaryotic cells (a review). Free Radic Res Commun. 17(4):221-237 (1992).
[128] Swanson J A, Hoppe A D. The coordination of signaling during Fc receptor-mediated phagocytosis. J Leukoc Biol. 76(6):1093-1103 (2004).
[129] Yamamoto K, Johnston R B Jr. Dissociation of phagocytosis from stimulation of the oxidative metabolic burst in macrophages. J Exp Med. 159(2):405-416 (1984).
[130] Debets J M, Van der Linden C J, Dieteren I E, Leeuwenberg J F, Buurman W A. Fc-receptor cross-linking induces rapid secretion of tumor necrosis factor (cachectin) by human peripheral blood monocytes. J. Immunol. 141(4):1197-1201 (1988).
[131] Weinberg J A, McGwin G Jr, Marques M B, et al. Transfusions in the less severely injured: does age of transfused blood affect outcomes? J Trauma. 65(4):794-798 (2008).
[132] Vandromme M J, McGwin G Jr, Marques M B, Kerby J D, Rue L W 3rd, Weinberg J A. Transfusion and pneumonia in the trauma intensive care unit: an examination of the temporal relationship. J Trauma. 67(1):97-101 (2009).
[133] Ness P M. Does transfusion of stored red blood cells cause clinically important adverse effects? A critical question in search of an answer and a plan. Transfusion. 2011; 51(4):666-667.
[134] Pettila V, Westbrook A J, Nichol A D, et al. Age of red blood cells and mortality in the critically ill. Crit. Care; 15(2):R116.
[135] Sanders J, Patel S, Cooper J, et al. Red blood cell storage is associated with length of stay and renal complications after cardiac surgery. Transfusion. May 12, 2011. doi: 10.1111/j.1537-2995.2011.03170.x. [Epub ahead of print].
[136] D'Alessandro A, Liumbruno G, Grazzini G, Zolla L. Red blood cell storage: the story so far. Blood Transfus. 2010; 8(2):82-88.
[137] Kekre N, Chou A, Tokessey M, et al. Storage time of transfused red blood cells and impact on clinical outcomes in hematopoietic stem cell transplantation. Transfusion. May 13, 2011. doi: 10.1111/j.1537-2995.2011.03166.x. [Epub ahead of print].
[138] Lelubre C, Piagnerelli M, Vincent J L. Association between duration of storage of transfused red blood cells and morbidity and mortality in adult patients: myth or reality? Transfusion. 2009; 49(7):1384-1394.
[139] Offner P J, Moore E E, Biffl W L, Johnson J L, Silliman C C. Increased rate of infection associated with transfusion of old blood after severe injury. Arch Surg. 2002; 137(6):711-716.
[140] Hassan M, Pham T N, Cuschieri J, et al. The association between the transfusion of older blood and outcomes after trauma. Shock. 2011; 35(1):3-8.
[141] Whitaker B. Report of the US Department of Health and Human Services. The 2009 National Blood Collection and Utilization Survey Report. Washington, D.C.: US Department of Health and Human Services, Office of the Assistant Secretary for Health, 2011.
[142] Bennett-Guerrero E, Veldman T H, Doctor A, et al. Evolution of adverse changes in stored RBCs. Proc Natl Aced Sci USA. 2007; 104(43):17063-17068.
[143] Hod E A, Zhang N, Sokol S A, et al. Transfusion of red blood cells after prolonged storage produces harmful effects that are mediated by iron and inflammation. Blood. 2010; 115(21):4284-4292.
[144] Hod E A, Hendrickson J E, Sharma S, et al. Transfusion of Mouse RBCs Stored in AS-1 Induces Inflammation and Exacerbates Bacterial Sepsis. Transfusion. 2010; 50(Suppl):523-010D.
[145] Bratosin D, Mazurier J, Tissier J P, et al. Cellular and molecular mechanisms of senescent erythrocyte phagocytosis by macrophages. A review. Biochimie. 1998; 80(2):173-195.
[146] Hutchinson C, Al-Ashgar W, Liu D Y, Hider R C, Powell J J, Geissler C A. Oral ferrous sulphate leads to a marked increase in pro-oxidant nontransferrin-bound iron. Eur J Clin Invest. 2004; 34(11):782-784.
[147] Breuer W, Hershko C, Cabantchik Z I. The importance of non-transferrin bound iron in disorders of iron metabolism. Transfus Sci. 2000; 23(3):185-192.
[148] Ratledge C. Iron metabolism and infection. Food Nutr Bull. 2007; 28(4 Suppl):S515-523.
[149] Nairz M, Schroll A, Sonnweber T, Weiss G. The struggle for iron—a metal at the host-pathogen interface. Cell Microbiol. 2010; 12(12):1691-1702.
[150] Krewulak K D, Vogel H J. Structural biology of bacterial iron uptake. Biochim Biophys Acta. 2008; 1778(9):1781-1804.
[151] Walter P B, Fung E B, Killilea D W, et al. Oxidative stress and inflammation in ironoverloaded patients with beta-thalassaemia or sickle cell disease. Br J Haematol. 2006; 135(2):254-263.
[152] de Valk B, Addicks M A, Gosriwatana I, Lu S, Hider R C, Marx J J. Non-transferrinbound iron is present in serum of hereditary haemochromatosis heterozygotes. Eur J Clin Invest. 2000; 30(3):248-251.
[153] Riddington C, Owusu-Ofori S. Prophylactic antibiotics for preventing pneumococcal infection in children with sickle cell disease. Cochrane Database Syst Rev. 2002(3):CD003427.
[154] Ricerca B M, Di Girolamo A, Rund D. Infections in thalassemia and hemoglobinopathies: focus on therapy-related complications. Mediterr J Hematol Infect Dis. 2009; 1(1):e2009028.
[155] Khan F A, Fisher M A, Khakoo R A. Association of hemochromatosis with infectious diseases: expanding spectrum. Int J Infect Dis. 2007; 11(6):482-487.
[156] Dresow B, Petersen D, Fischer R, Nielsen P. Non-transferrin-bound iron in plasma following administration of oral iron drugs. Biometals. 2008; 21(3):273-276.
[157] Sazawal S, Black R E, Ramsan M, et al. Effects of routine prophylactic supplementation with iron and folic acid on admission to hospital and mortality in preschool children in a high malaria transmission setting: community-based, randomised, placebo-controlled trial. Lancet. 2006; 367(9505):133-143.
[158] Conclusions and recommendations of the WHO Consultation on prevention and control of iron deficiency in infants and young children in malaria-endemic areas. Food Nutr Bull. 2007; 28(4 Suppl):S621-627.
[159] The transfusion of red cells. In: Klein H G, Antsee D J, eds. Mollison's Blood Transfusion in Clinical Medicine, 11th edition: Blackwell Publishing Ltd; 2005:382.
[160] Wiesen A R, Hospenthal D R, Byrd J C, Glass K L, Howard R S, Diehl L F. Equilibration of hemoglobin concentration after transfusion in medical inpatients not actively bleeding. Ann Intern Med. 1994; 121(4):278-230.
[161] Sekhsaria S, Fomufod A. Readjustment of hematocrit values after packed red cell transfusion in the neonate. J Perinatol. 1991; 11(2):161-163.
[162] Hod E A, Spitalnik S L. Harmful effects of transfusion of older stored red blood cells: iron and inflammation. Transfusion. 2011; 51(4):881-885.
[163] Matinaho S, von Bonsdorff L, Rouhiainen A, Lonnroth M, Parkkinen J. Dependence of Staphylococcus epidermidis on non-transferrin-bound iron for growth. FEMS Microbiol Lett. 2001; 196(2):177-182.
[164] Gladwin M T, Kim-Shapiro D B. Storage lesion in banked blood due to hemolysisdependent disruption of nitric oxide homeostasis. Curr Opin Hematol. 2009; 16(6):515-523.
[165] Lee J S, Gladwin M T. Bad blood: the risks of red cell storage. Nat. Med. 2010; 16(4):381-382.
[166] Donadee C, Raat N J, Kanias T, et al. Nitric oxide scavenging by red blood cell microparticles and cell-free hemoglobin as a mechanism for the red cell storage lesion. Circulation. 2011; 124(4):465-476.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

What is claimed is:

1. An apparatus for ameliorating an adverse effect in a patient caused by an acute transfusion into the patient of a composition comprising aged red blood cells, the apparatus comprising an inner surface that is in sterile contact with the composition and an effective amount of an iron chelator.

2. The apparatus according to claim 1, wherein the iron chelator is disposed on the inner surface of the apparatus that is in sterile contact with the composition.

3. The apparatus according to claim 1, wherein the iron chelator is disposed within an inner space of the apparatus formed by the inner surface that is in sterile contact with the composition.

4. The apparatus according to claim 1, which is a container for storing red blood cells for transfusion into a patient in need thereof.

5. The apparatus according to claim 4, wherein the container is a blood transfusion bag.

6. The apparatus according to claim 1, wherein the apparatus is a blood filter.

7. The apparatus according to claim 1, wherein the iron chelator is selected from the group consisting of apotransferrin, lactotransferrin, metalloenzymes, an hydroxamic acid polymer, a phosphorylated myo-inositol polymer, heme B, heme A, heme C, desferoxamine (DFO), desferrithiocin (DFT), desferri-exochelin (D-Exo), (S)-DMFT, (S)-DADMDFT, (S)-DADFT, 4′-(OH)-DADFT, 4′-(OH)-DADMDFT or its hexadentate derivative BDU, deferiprone (L1), an hydroxypyridinone ester prodrug whose metabolism yields a hydroxypyridinone analog, CP94, CP502, CP365, CP102, CP41, CP38, LiNAII, Pr-(Me-3,2-HOPO) and its hexadentate analog TREN-(Me-3,2-HOPO), CP117, CP165, tachpyridine alkyl analogs, tachpyridine secondary amine linked analogs, tachpyridine pyridyl linked analogs, tachpyridine pyridyl linked maleimide derivative analogs, PIH, SIH, PCIH, PKIH, PIH analog compound 101, PIH analog compound 102, PIH analog compound 103, PIH analog compound 104, PIH analog compound 105, PIH analog compound 106, PIH analog compound 107, PIH analog compound 108, PIH analog compound 109, PIH analog compound 110, PIH analog compound 112, PIH analog compound 113, PIH analog compound 114, PIH analog compound 115, PIH analog compound 201, PIH analog compound 202, PIH analog compound 204, PIH analog compound 205, PIH analog compound 206, PIH analog compound 207, PIH analog compound 208, PIH analog compound 209, PIH analog compound 212, PIH analog compound 215, PIH analog compound 301, PIH analog compound 302, PIH analog compound 305, PIH analog compound 307, PIH analog compound 308, PIH analog compound 309, PIH analog compound 310, PIH analog compound 312, PIH analog compound 315, PCBH, PCHH, PCBBH, PCAH, PCTH, PKBH, PKAH, PK3BBH, PKHH, PKTH, 5-HP, Triapine, NT, N2 mT, N4 mT, N44 mT, N4eT, N4aT, N4pT, DpT, DP2 mT, Dp4 mT, Dp44 mT, Dp4eT, Dp4aT, Dp4pT, deferasirox (Exjade, ICL670A), a 5,5-diphenyl-1,2,4-triazole analog of deferasirox, HBED, Faralex-G, 4-hydroxy-2-nonylquinoline, and combinations thereof.

8. The apparatus according to claim 7, wherein the iron chelator is selected from the group consisting of desferoxamine, deferasirox, and apotransferrin.

9. A kit for ameliorating an adverse effect in a patient caused by an acute transfusion into the patient of a composition comprising aged red blood cells, the kit comprising a container comprising an effective amount of an iron chelator packaged together with instructions on how to administer the iron chelator to the composition directly, to a blood product-related apparatus, or to a patient in need thereof.

10. The kit according to claim 9, wherein the blood product-related apparatus is a blood filter or a blood bag.

11. The kit according to claim 9, wherein the iron chelator is selected from the group consisting of apotransferrin, lactotransferrin, metalloenzymes, an hydroxamic acid polymer, a phosphorylated myo-inositol polymer, heme B, heme A, heme C, desferoxamine (DFO), desferrithiocin (DFT), desferri-exochelin (D-Exo), (S)-DMFT, (S)-DADMDFT, (S)-DADFT, 4′-(OH)-DADFT, 4′-(OH)-DADMDFT or its hexadentate derivative BDU, deferiprone (L1), an hydroxypyridinone ester prodrug whose metabolism yields a hydroxypyridinone analog, CP94, CP502, CP365, CP102, CP41, CP38, LiNAII, Pr-(Me-3,2-HOPO) and its hexadentate analog TREN-(Me-3,2-HOPO), CP117, CP165, tachpyridine alkyl analogs, tachpyridine secondary amine linked analogs, tachpyridine pyridyl linked analogs, tachpyridine pyridyl linked maleimide derivative analogs, PIH, SIH, PCIH, PKIH, PIH analog compound 101, PIH analog compound 102, PIH analog compound 103, PIH analog compound 104, PIH analog compound 105, PIH analog compound 106, PIH analog compound 107, PIH analog compound 108, PIH analog compound 109, PIH analog compound 110, PIH analog compound 112, PIH analog compound 113, PIH analog compound 114, PIH analog compound 115, PIH analog compound 201, PIH analog compound 202, PIH analog compound 204, PIH analog compound 205, PIH analog compound 206, PIH analog compound 207, PIH analog compound 208, PIH analog compound 209, PIH analog compound 212, PIH analog compound 215, PIH analog compound 301, PIH analog compound 302, PIH analog compound 305, PIH analog compound 307, PIH analog compound 308, PIH analog compound 309, PIH analog compound 310, PIH analog compound 312, PIH analog compound 315, PCBH, PCHH, PCBBH, PCAH, PCTH, PKBH, PKAH, PK3BBH, PKHH, PKTH, 5-HP, Triapine, NT, N2 mT, N4 mT, N44 mT, N4eT, N4aT, N4pT, DpT, DP2 mT, Dp4 mT, Dp44 mT, Dp4eT, Dp4aT, Dp4pT, deferasirox (Exjade, ICL670A), a 5,5-diphenyl-1,2,4-triazole analog of deferasirox, HBED, Faralex-G, 4-hydroxy-2-nonylquinoline, and combinations thereof.

12. The kit according to claim 11, wherein the iron chelator is selected from the group consisting of desferoxamine, deferasirox, and apotransferrin.

13. A method for ameliorating an adverse effect in a patient caused by an acute transfusion into the patient of a composition comprising aged red blood cells, the method comprising providing an iron chelator, which is capable of chelating iron released by macrophage phagocytosis of the aged red blood cells, wherein the chelator ameliorates the adverse effect in the patient.

14. The method according to claim 13, wherein the adverse effect is a cytokine storm.

15. The method according to claim 13, wherein the adverse effect is an increase in iron-dependent pathogens in the patient.

16. The method according to claim 13, wherein the iron chelator is selected from the group consisting of peptides, polymers, small organic or inorganic molecules and combinations thereof.

17. The method according to claim 16, wherein the iron chelating peptide is selected from the group consisting of apotransferrin, lactotransferrin, metalloenzymes, iron-binding domains from such proteins, and synthetic peptides designed to mimic the iron-binding site of such proteins.

18. The method according to claim 16, wherein the iron chelating polymer is an hydroxamic acid polymer or a phosphorylated myo-inositol polymer.

19. The method according to claim 13, wherein the iron chelator is a porphyrin ring selected from the group consisting of heme B, heme A, and heme C.

20. The method according to claim 13, wherein the iron chelator is a siderophore or a synthetically derived analog thereof.

21. The method according to claim 20, wherein the siderophore is selected from the group consisting of desferoxamine (DFO), desferrithiocin (DFT), and desferri-exochelin (D-Exo).

22. The method according to claim 13, wherein the iron chelator is a DFT analog selected from the group consisting of (S)-DMFT, (S)-DADMDFT, (S)-DADFT, 4′-(OH)-DADFT, and 4′-(OH)-DADMDFT or its hexadentate derivative BDU.

23. The method according to claim 13, wherein the iron chelator is a hydroxypyridinone.

24. The method according to claim 23, wherein the hydroxypyridinone is selected from deferiprone (L1) or its analogs or an hydroxypyridinone ester prodrug whose metabolism yields a hydroxypyridinone analog.

25. The method according to claim 24, wherein the deferiprone analog is selected from the group consisting of CP94, CP502, CP365, CP102, CP41, CP38, LiNAII, Pr-(Me-3,2-HOPO) and its hexadentate analog TREN-(Me-3,2-HOPO).

26. The method according to claim 24, wherein the hydroxypyridinone ester prodrug is selected from the group consisting of CP117 and CP165.

27. The method according to claim 13, wherein the iron chelator is a tachpyridine or an analog thereof.

28. The method according to claim 27, wherein the tachpyridine analog is selected from the group consisting of tachpyridine alkyl analogs, tachpyridine secondary amine linked analogs, tachpyridine pyridyl linked analogs, and tachpyridine pyridyl linked maleimide derivative analogs.

29. The method according to claim 13, wherein the iron chelator is an aroylhydrazone.

30. The method according to claim 29, wherein the aroylhydrazone iron chelator is selected from the group consisting of PIH, SIH, 311 series analog compounds, PCIH, PKIH, and analogs of each parent compound.

31. The method according to claim 30, wherein the PIH analog is selected from the group consisting of 100 series analog compounds 101, 102, 103, 104, 105, 106, 107, 108, 109, 110, 112, 113, 114 and 115.

32. The method according to claim 30, wherein the PIH analog is selected from the group consisting of 200 series analog compounds 201, 202, 204, 205, 206, 207, 208 209, 212, and 215.

33. The method according to claim 30, wherein the 311 series analog compounds are selected from the group consisting of compounds 301, 302, 305, 307 308, 309, 310, 312, and 315.

34. The method according to claim 30, wherein the PCIH analogs are selected from the group consisting of PCBH, PCHH, PCBBH, PCAH and PCTH.

35. The method according to claim 30, wherein the PKIH analogs are selected from the group consisting of PKBH, PKAH, PK3BBH, PKHH, and PKTH.

36. The method according to claim 13, wherein the iron chelator is a thiosemicarbazone.

37. The method according to claim 36, wherein the thiosemicarbazone is selected from the group consisting of 5-HP, Triapine, members of the NT series, and members of the DpT series.

38. The method according to claim 37 wherein the NT series is selected from the group consisting of NT, N2 mT, N4 mT, N44 mT, N4eT, N4aT, and N4pT.

39. The method according to claim 37, wherein the DpT series is selected from the group consisting of DpT, DP2 mT, Dp4 mT, Dp44 mT, Dp4eT, Dp4aT, and Dp4pT.

40. The method according to claim 13, wherein the iron chelator is selected from the group consisting of deferasirox (Exjade, ICL670A), a 5,5-diphenyl-1,2,4-triazole analog of deferasirox, HBED, Faralex-G, and 4-hydroxy-2-nonylquinoline.

41. The method according to claim 13, wherein the providing step comprises administering to the patient an amount of the iron chelator that is effective to ameliorate the adverse effect.

42. The method according to claim 13, wherein the providing step comprises, prior to transfusion, contacting the composition comprising aged red blood cells with an amount of the iron chelator that is effective to ameliorate the adverse effect.

43. The method according to claim 13, wherein the patient is a human.
